(19) United States
(12) Patent Application Publication
(10) Pub. No.: US 2015/0010610 A1
(43) Pub. Date: Jan. 8, 2015

(54) IMMUNOCOMPATIBLE AMNIOTIC MEMBRANE PRODUCTS

(71) Applicant: OSIRIS THERAPEUTICS, INC., Columbia, MD (US)

(72) Inventors: Samson Tom, Basking Ridge, NJ (US); Alla Danilkovitch, Columbia, MD (US); Dana Yoo, Falls Church, VA (US); Timothy Jansen, Baltimore, MD (US); Jin-Qiang Kuang, Woodstock, MD (US); Jennifer Michelle Marconi, Glen Burnie, MD (US)

(73) Assignee: OSIRIS THERAPEUTICS, INC., Columbia, MD (US)

(21) Appl. No.: 14/272,345

(22) Filed: May 7, 2014

Related U.S. Application Data
(63) Continuation-in-part of application No. 14/172,940, filed on Feb. 5, 2014.
Application No. 14/172,940 is a continuation of application No. 13/030,551, filed on Feb. 18, 2011, now abandoned.
(60) Provisional application No. 61/338,464, filed on Feb. 18, 2010.
(60) Provisional application No. 61/338,489, filed on Feb. 18, 2010.
(60) Provisional application No. 61/369,562, filed on Jul. 30, 2010.

(51) Int. Cl. A61K 35/50; A61L 27/36; A61L 27/54

(52) U.S. Cl. CPC A61K 35/50; A61L 27/54; A61L 27/3604; A61K 2035/122
USPC 424/423; 435/1.1; 424/93.7

(57) ABSTRACT

Provided herein is a placental membrane product comprising an immunocompatible amniotic membrane. Such placental membrane products can be cryopreserved and contain therapeutic factors and viable cells after thawing. The placental membrane products are useful in wound healing and tissue repair/regeneration as they are capable of promoting angiogenesis, reducing inflammation, inhibiting proteases and free radical oxidation, reducing scar formation, and other methods that promote healing. The present technology relates to products to protect injured or damaged tissue, or as a covering to prevent adhesions, to exclude bacteria, to inhibit bacterial activity, and/or to promote healing or growth of tissue. The field also relates to methods of manufacturing and methods of use of such membrane-derived products.

### RELATED APPLICATIONS

This application is a continuation in part application of U.S. application Ser. No. 14/172,940, filed Feb. 5, 2014, which is a continuation of U.S. application Ser. No. 13/030,551, filed Feb. 18, 2011. The above applications claim the benefit of priority to:

U.S. Provisional Application Ser. No. 61/338,464 entitled “Selectively Immunodepleted Chorionic Membranes”, filed on Feb. 18, 2010 bearing Docket No. 22924US01,

U.S. Provisional Application Ser. No. 61/338,489 entitled “Selectively Immunodepleted Amniotic Membranes”, filed on Feb. 18, 2010 bearing Docket No. 22925US01, and

U.S. Provisional Application Ser. No. 61/369,562 entitled “Therapeutic Products Comprising Vitalized Placental Dispersions filed on Jul. 30, 2010 bearing Docket No 23498US01. The contents of all the above applications are hereby incorporated by reference in their entireties.

This application is being co-filed on May 7, 2014 with, and incorporates by reference in their entireties, applications entitled:

“Immunocompatible Chorionic Membrane Products”, and

Therapeutic Placental Compositions, Methods Of Making And Methods Of Use.

### FIELD OF THE INVENTION

The present technology relates to methods and products that facilitate or improve wound healing including, for example placenta membrane-derived products and cryopreserved products, and methods that promote healing in a wound or near or at the site of a wound such as combinations of one or more of the following: stimulation of angiogenesis, secretion of growth factors, inhibition of proteases and free radical oxidation. The present technology relates to products to protect injured or damaged tissue, or as a covering to prevent adhesions, to exclude bacteria, to inhibit bacterial activity, or to promote healing or growth of tissue. An example of such a placental membrane is an amniotic membrane. The field also relates to methods of manufacturing and methods of use of such membrane-derived products.

### BACKGROUND OF THE INVENTION

Fresh or decellularized placental membranes have been used topically in surgical applications since at least 1910 when Johns Hopkins Hospital reported the use of placental membrane for dermal applications. Subsequently unseparated amnion and chorion were used to treat burned or ulcerated surfaces. During the 1950's and 60's Troensegaard-Hansen applied boiled amniotic membranes to chronic leg ulcers.

The human amniotic membrane (AM) is the innermost of the fetal membranes deriving from the amniotic sac and constituting the lining of the amniotic cavity. It is approximately 0.02 to 0.5 mm thick. The AM consists of five layers: a single layer of epithelial cells rests on the basement membrane and contacts the amniotic fluid. An underlying layer of connective tissue is attached to the basement membrane. The connective tissue is comprised of three structural layers: a compact layer, a fibroblast layer (sometimes referred to as a mesenchymal layer), and a spongy layer. The spongy layer is adjacent to the chorion. The amnion is essentially devoid of vasculature.

Both fresh and frozen AMs have been used for wound healing therapy. The AM contains a number of factors that can contribute to wound healing such as, for example, extracellular matrix, growth factors, and viable cells. While some preserving methods can maintain some level of factors such as matrix or growth factors, preserving levels of viable cells presents a challenge. When fresh AM is used, there is increased risk of disease transmission. According to published reports, fresh amniotic tissue exhibits high cell viability (e.g., greater than 70%), however cell viability rapidly diminishes during storage (e.g., within 28 days at temperatures above 0° C.), dropping the amount of viable cells to ranges of about 15%-35%. Freezing for a period of time (e.g., 3 weeks) was also shown to reduce cell viability to ranges of about 13% to 18%, regardless of the temperature or medium.

Lee and Tseng report the cryopreservation of AM in glycerol and Dulbeccos Modified Eagle medium (DMEM) at −80° C., although such cryopreservation dramatically decreases cell viability. According to published reports, glycerol storage of AM resulted in immediate cell death. Glycerol cryopreserved AM (−80° C.) and glycerol-preserved AM (−4° C.) are sufficient to provide a matrix for wound healing, but fail to provide sufficient cell viability to bestow biological effectiveness.

Gajiwala and Gajiwala report the preservation of AM by freeze-drying (lyophilisation) and gamma-irradiation sterilization. According to this method, AM is pasteurized at 60° C., treated with 70% ethanol, and freeze-dried to remove most of the remaining moisture. Then the AM is sterilized by exposure of 25 kGy gamma-radiation in a Cobalt 60 Gamma chamber unit or at an ISO-certified radiation plant. The sterilized AM can be stored at room temperature for up to 6 months.

Gomes reports preservation of AM with lyophilisation followed by sterilization in ethylene oxide.

Rama et al reported the cryopreservation of AM in 10% dimethyl sulfoxide (DMSO) instead of glycerol and achieved a cell viability of about 40%.

There are currently two commercially available bioengineered tissue graft products which contain living cells(derived from neonatal foreskin), Apligraf and Dermagraft. Both Apligraf and Dermagraft comprise cultured-expanded cells. Neither Apligraf nor Dermagraft comprise detectable levels of certain factors such as, for example, Insulin-like Growth Factor Binding Protein-1 (IGFBP-1) and adiponectin, which are factors associated with the natural wound healing process. In addition, neither Apligraf nor Dermagraft exhibit certain ratios of matrix metalloproteinases (MMPs) to tissue inhibitor of matrix metalloproteinase (TIMPS) (MMP to TIMP ratio or protease-to-protease inhibitor ratio) that may be favorable for wound healing. As wound healing is a multi-factorial biological process, many factors are needed to properly treat a wound; products having lower amounts of viable cells and limited diversity compared to the cells present in skin are less capable of healing wounds relative to a product having an increased population of viable cells and increased number of types of cells found in tissues. It would represent an advance in the art to provide an amnion-derived product that can be used in applications such as wound healing, wound dressings, cosmetic uses, or as a biologic skin substitute comprising a diverse population of cells representing a higher percentage of viable cells and an increased amount of factors, including, for example, growth factors, antioxidant agents, anti-inflammatory agents, agents that promote angiogenesis and cytokines.

Apligraf is a living, bi-layered skin substitute manufactured using neonatal foreskin keratinocytes and fibroblasts combined with bovine Type I collagen. As used in this application, Apligraf refers to the product available for commercial sale as approved by the FDA in 1998.

Dermagraft is cryopreserved human fibroblasts derived from newborn foreskin tissue seeded on a synthetic extracellular matrix, and a bioabsorbable polyglactin mesh scaffold. According to its product literature, Dermagraft requires three washing steps before use which limits the practical implementation of Dermagraft as a wound dressing relative to products that require less than three washing steps. As used in this application, Dermagraft refers to the product available for commercial sale as approved by the FDA in 2001.

Engineered wound dressings such as Apligraf and Dermagraft do not provide the best potential for wound healing because they comprise sub-optimal cellular compositions and therefore do not provide proper wound healing. For example, Apligraf and Dermagraft do not comprise MSCs or inherent tissue extracellular matrix and, as a result, the ratio between different factors secreted by cells does not enable efficient wound healing. Additionally, some factors that are important for wound healing, including EGF, IGFBP1, and adiponectin, are not detectable or are absent from both Apligraf and Dermagraft. Additionally, some factors, including MMPs and TIMPs, are present in proportions that differ greatly from the proportions found in the natural wound healing process; this difference significantly alters, among other things, upstream inflammatory cytokine pathways which in turn allow for sub-optimal micro-environments at the wound site. The matrix composition in these bioengineered products includes only Collagen type I and may also include hyaluronic acid. This differs from the complex structural matrix of skin which includes components such as various collagens (e.g., collagens I, Ill, IV, V, VI, etc.), elastin, glycoproteins and proteoglycans. Skin also includes mesenchymal stem cells in the dermis, which are lacking in the representative examples of bioengineered products, Apligraf and Dermagraft.

Paquet-Fifield et al. report that mesenchymal stem cells and fibroblasts are important for wound healing (J Clin Invest, 2009, 119: 2795). No product has yet been described that comprises mesenchymal stem cells and fibroblasts.

Both MMPs and TIMPs are among the factors that are important for wound healing. However, expression of these proteins must be highly regulated and coordinated. Excess of MMPs versus TIMPs is a marker of poor chronic wound healing (Liu et al, Diabetes Care, 2009, 32: 117; Mwaura et al, Eur J Vasc Endovasc Surg, 2006, 31: 306; Trengove et al, Wound Rep Reg, 1999, 7: 442; Vaalamo et al, Hum Pathol, 1999, 30: 795).

α2-macroglobulin and/or its receptor is known as a plasma protein that inactivates proteases from all four mechanistic classes: serine proteases, cysteine proteases, aspartic proteases, and metalloproteases (Borth et al., FASEB J, 1992, 6: 3345; Baker et al., J Cell Sci, 2002, 115:3719). Another important function of this protein is to serve as a reservoir for cytokines and growth factors, examples of which include TGF, PDGF, and FGF (Asplin et al, Blood, 2001, 97: 3450; Huang et al, J Biol Chem, 1988; 263: 1535). In chronic wounds like diabetic ulcers or venous ulcers, the presence of high amount of proteases leads to rapid degradation of growth factors and delays in wound healing. Thus, a placental product comprising α2-macroglobulin would constitute an advance in the art.

An in vitro cell migration assay is important for assessing the wound healing potential of a product. The process of wound healing is highly complex and involves a series of structured events controlled by growth factors (Goldman, Adv Skin Wound Care, 2004, 1:24). These events include increased vascularization, infiltration by inflammatory immune cells, and increases in cell proliferation. The beginning stages of wound healing revolve around the ability of individual cells to polarize towards the wound and migrate into the wounded area in order to close the wound area and rebuild the surrounding tissue. An assay capable of evaluating the wound healing potential of wound therapies by examining the correlation between cell migration and wound healing would represent an advance in the art. As discussed in the disclosure that follows, aspects of the present technology represent a significant advance in the art as they relate to products and methods that promote angiogenesis, promote anti-inflammatory activity, promote antioxidant activity, and provide for increased amounts and varieties of growth factors. As discussed in more detail below, these products and methods can be used in any number of wound healing applications, soft tissue repair, or osteogenic repair.

### SUMMARY OF THE INVENTION

In one aspect, the disclosure provides a membrane comprising cryopreserved amniotic membrane, wherein after cryopreservation and subsequent thawing the amniotic membrane comprises:

A) tissue cells, wherein said tissue cells are native to the amniotic membrane and greater than about 40% of said tissue cells are viable;

B) one or more therapeutic factors that are native to the amniotic membrane;

C) extracellular matrix that is native to the amniotic membrane; and

D) depleted amounts of one or more types of functional immunogenic cells.

In another aspect, the disclosure provides a membrane comprising cryopreserved amniotic membrane, wherein after cryopreservation and subsequent thawing the amniotic membrane comprises:

A) a stromal layer comprising viable cells, one or more therapeutic factors, and extracellular matrix,

B) an epithelial layer comprising viable cells and one or more therapeutic factors; and

C) depleted amounts of one or more types of functional immunogenic cells.

wherein greater than 40% of the combined cells in the stromal layer and epithelial layer are viable cells.

In one aspect, the disclosure provides a membrane comprising cryopreserved amniotic membrane having one or more tissue components, wherein after cryopreservation and subsequent thawing the one or more tissue components comprise:

A) tissue cells, wherein said tissue cells are native to the amniotic membrane and greater than 40% of said tissue cells are viable;

B) one or more therapeutic factors that are native to the amniotic membrane; and

C) extracellular matrix that is native to the amniotic membrane;

wherein the amniotic membrane has depleted amounts of types of functional immunogenic cells; and wherein the one or more tissue components is present in an amount effective to provide a therapeutic benefit.

In one aspect, the disclosure provides a membrane comprising cryopreserved amniotic membrane having one or more tissue components, wherein after cryopreservation and subsequent thawing the one or more tissue components comprise:

A) tissue cells, wherein said tissue cells are native to the amniotic membrane and greater than 40% of said tissue cells are viable;

B) one or more therapeutic factors that are native to the amniotic membrane; and

C) extracellular matrix that is native to the amniotic membrane;

wherein the amniotic membrane has depleted amounts of functional immunogenic cells, and wherein the one or more tissue components is present in an amount effective to:

(i) reduce the amount and/or activity of pro-inflammatory cytokines;

(ii) increase the amount and/or activity of anti-inflammatory cytokines;

(iii) reduce the amount and/or activity of reactive oxygen species;

(iv) increase the amount and/or activity of antioxidant agents;

(v) reduce the amount and/or activity of proteases;

(vi) increase cell proliferation;

(vii) increase angiogenesis; and/or

(viii) increase cell migration.

In another aspect, the disclosure provides a membrane comprising cryopreserved placental membrane, wherein after cryopreservation and subsequent thawing the placental membrane comprises:

A) tissue cells, wherein said tissue cells are native to the placental membrane and greater than 40% of said tissue cells are viable;

B) one or more therapeutic factors that are native to the placental membrane;

C) extracellular matrix that is are native to the placental membrane; and

D) depleted amounts of one or more types of functional immunogenic cells.

In some embodiments of this aspect, the placental membrane comprises amniotic membrane and chorionic membrane.

In some embodiments of the above aspects, the depleted amounts of functional immunogenic cells produce immunogenic factors in amounts that are below levels sufficient to produce an immune response. In some embodiments, the depleted amounts of functional immunogenic cells produce immunogenic factors in amounts below detectable limits.

In embodiments of the above aspects, the membrane comprises tissue cells wherein about 50% to about 100% of said tissue cells are viable. In some embodiments, about 60% to about 100% of said tissue cells are viable. In further embodiments, about 70% to about 100% of said tissue cells are viable.

In various embodiments of the above aspects, the membranes provide one or more tissue components (e.g., viable cells, one or more therapeutic factors, and/or extracellular matrix) in an amount that is effective to promote any of the activities of (i)-(viii) in vitro or in vivo.

Various embodiments of the above-described aspects may further comprise a delivery substrate, such that the membrane is fixed to the delivery substrate.

In embodiments of the above aspects, the membrane may be stored for an extended period of time prior to subsequent thawing. In some embodiments the extended period of time is from about 6 months to about 25 months or more. In these embodiments, the viability of the tissue cells is substantially maintained upon thawing. In some embodiments, the viability of the tissue cells is substantially maintained for at least about 25 months or more when stored frozen.

In embodiments of the above aspects, the membrane can be thawed and ready for use within 30 minutes of the start of a thawing method.

In some embodiments of the above aspects, the membrane can be stored in saline up to an hour after thawing and still maintain about 70% viable cells.

In another aspect, the disclosure provides a method of treating a wound on a subject comprising administering a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein upon administration the membrane provides viable cells, extracellular matrix, and/or one or more therapeutic factors in an amount effective to promote one or more of:

(i) a reduction of the amount and/or activity of pro-inflammatory cytokines;

(ii) an increase in the amount and/or activity of anti-inflammatory cytokines;

(iii) a reduction of the amount and/or activity of reactive oxygen species;

(iv) an increase in the amount and/or activity of antioxidant agents;

(v) a reduction of the amount and/or activity of proteases;

(vi) an increase in cell proliferation;

(vii) an increase in angiogenesis; and/or

(viii) an increase in cell migration.

In another aspect, the disclosure provides a method for accelerating wound healing comprising administering a membrane according to any of the aspects and embodiments described herein. In some embodiments, the administering is effective to promote wound closure by 12 weeks after an initial administering step. In some embodiments, the administering is effective to promote wound closure by 5-6 weeks after an initial administering step. In some embodiments, the administering is effective to promote reduction in wound size by 50% or more 28 days after an initial administering step. In embodiments, the administering is effective to improve wound closure rate by at least about 44% relative to standard wound treatment.

In another embodiment, the disclosure provides method of treating a subject for a wound that is refractory to a prior wound healing treatment, the method comprising administering to the site of the wound a membrane according to any of the aspects and embodiments described herein. In some embodiments, the administering is effective to promote wound closure by 12 weeks after an initial administering step. In some embodiments, the administering is effective to promote wound closure by 5-6 weeks after an initial administering step. In some embodiments, the administering is effective to promote reduction in wound size by 50% or more 28 days after an initial administering step.

In another aspect, the disclosure provides a method for treating a chronic wound comprising administering a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that are native to the amniotic membrane; and

D) depleted amounts of one or more types of functional immunogenic cells;

wherein the administering provides the viable therapeutic cells, extracellular matrix, and one or more therapeutic factors in an amount effective to promote healing of the chronic wound.

In another aspect, the disclosure provides a method for treating an acute wound comprising administering a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that are native to the amniotic membrane; and

D) depleted amounts of one or more types of functional immunogenic cells;

wherein the administering provides the viable therapeutic cells, extracellular matrix, and one or more therapeutic factors in an amount effective to promote healing of the acute wound.

In embodiments of the above aspects relating to wound treatment, accelerated wound treatment, and/or chronic wound treatment, the wound may be selected from the group consisting of lacerations, scrapes, burns, incisions, punctures, wound caused by a projectile, an epidermal wound, skin wound, chronic wound, acute wound, external wound, internal wound, congenital wound, ulcer, pressure ulcer, diabetic ulcer, tunnel wound, wound caused during or as an adjunct to a surgical procedure, venous skin ulcer, and avascular necrosis.

In embodiments of the above aspects relating to methods, the membrane may either directly or may indirectly promote one or more of: (i) a reduction of the amount and/or activity of pro-inflammatory cytokines; (ii) an increase in the amount and/or activity of anti-inflammatory cytokines; (iii) a reduction of the amount and/or activity of reactive oxygen species; (iv) an increase in the amount and/or activity of antioxidant agents; (v) a reduction of the amount and/or activity of proteases; (vi) an increase in cell proliferation; (vii) an increase in angiogenesis; and/or (viii) an increase in cell migration.

In another aspect, the disclosure provides a method of treating an inflammatory ocular condition in a subject comprising administering to the subject a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable therapeutic cells, extracellular matrix, and one or more therapeutic factors in an amount effective to treat the inflammatory ocular condition.

In some embodiments of this aspect, the inflammatory ocular condition is a wound or a disease characterized by inflammation.

In another aspect, the disclosure provides a method of promoting tissue repair and/or tissue regeneration in a subject comprising administering to the subject a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable therapeutic cells, extracellular matrix, and one or more therapeutic factors in an amount effective to promote tissue repair and/or tissue regeneration.

In some embodiments of this aspect, the method is used in combination with a surgical procedure selected from the group consisting of a tissue graft procedure, tendon surgery, ligament surgery, bone surgery, and spinal surgery. In some embodiments, the tissue is human tissue. In further embodiments the human tissue is cartilage, skin, ligament, tendon, or bone. In this aspect and the various embodiments, the membrane may directly or indirectly stimulate tissue regeneration.

In another aspect, the disclosure provides a method of modulating inflammatory response comprising administering to the wound a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable cells, extracellular matrix, and/or one or more therapeutic factors in an amount effective to reduce the amount and/or activity of pro-inflammatory cytokines, and/or increase the amount and/or activity of anti-inflammatory cytokines.

In an aspect, the disclosure provides a method of modulating protease activity comprising administering to the wound a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable cells, extracellular matrix, and/or one or more therapeutic factors in an amount effective to reduce the amount and/or activity of a protease, and/or increase the amount and/or activity of protease inhibitors.

In a further aspect, the disclosure provides a method of reducing the amount and/or activity of reactive oxygen species (ROS) and increasing the amount and/or activity of antioxidant agents comprising administering to the wound a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable cells, extracellular matrix, and/or one or more therapeutic factors in an amount effective to reduce the amount and/or activity of ROS and/or increase the amount and/or activity of antioxidant agents.

In another aspect, the disclosure provides a method of increasing angiogenesis comprising administering to wound a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable cells, extracellular matrix, and/or one or more therapeutic factors in an amount effective to increase angiogenesis.

In another aspect, the disclosure provides a method of increasing cell migration comprising administering to wound a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable cells, extracellular matrix, and/or one or more therapeutic factors in an amount effective to increase cell migration.

In yet another aspect, the disclosure provides a method of increasing cell proliferation comprising administering to wound a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable cells, extracellular matrix, and/or one or more therapeutic factors in an amount effective to increase cell proliferation.

In an aspect, the disclosure provides a method of preventing or reducing scar or contracture formation in a subject comprising administering to the subject a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells;

wherein the administration provides the viable therapeutic cells, extracellular matrix, and one or more therapeutic factors in an amount effective to prevent or reduce scar or contracture formation.

In an aspect, the disclosure provides a method of treating or preventing tissue adhesion associated with a surgical procedure comprising administering a membrane comprising cryopreserved amniotic membrane, wherein after cryopreservation and subsequent thawing the amniotic membrane comprises:
A) tissue cells, wherein said tissue cells are native to the amniotic membrane and greater than 40% of said tissue cells are viable;
B) one or more therapeutic factors that are native to the amniotic membrane;
C) extracellular matrix that is native to the amniotic membrane; and
D) depleted amounts of one or more types of functional immunogenic cells;
   wherein the membrane is administered to the tissue and provides the viable cells, extracellular matrix, and/or one or more therapeutic factors in an amount effective to treat or prevent tissue adhesion.

In any of the above aspects relating to methods of modulating inflammatory response, modulating protease activity, reducing the amount and/or activity of reactive oxygen species, increasing the amount and/or activity of antioxidant agents, increasing/promoting angiogenesis, increasing cell migration, increasing cell proliferation, or preventing or reducing scar or contracture formation the administration of the membrane may directly or indirectly stimulate or induce the method.

In another aspect, the disclosure provides a kit for treating a wound or a tissue defect comprising:

A) a membrane according to any of the aspects and embodiments described herein, in a pharmaceutically acceptable container; and

B) instructions for administering the membrane for treating the wound or the tissue defect.

In embodiments, the kit may further comprise an additive. In further embodiments, the additive may be selected from one or more antibiotics, emollients, keratolytic agents, humectants, antioxidants, preservatives, therapeutics, bandages, tools, cutting device, buffer, thawing medium, handling media, forceps, container and combinations thereof.

The disclosure provides for other aspects and embodiments, which will be apparent from the description and non-limiting Examples that follow.

### BRIEF DESCRIPTION OF THE DRAWINGS

FIG. 1 depicts freezing rates of various freezing methods and various amounts of cryopreservation solution. FIG. 1A-1D involved a 30 minute refrigeration step at 4° C. before freezing at −80° C. FIG. 1E-1H show direct freezing at −80° C.

FIG. 2 depicts cell process recovery (A) and cell viability (B) of amniotic membrane when stored with various amounts of cryopreservation solution.

FIG. 3 depicts cell process recovery (A) and cell viability (B) of the chorionic membrane when stored with various amounts of cryopreservation solution.

FIG. 4 shows the effect of cryopreservation on cell viability of chorioamniotic membrane.

FIG. 5 depicts the effects of refrigeration time and freezing parameters on process (cryopreservation) cell recovery of amniotic (A) and chorionic (B) membrane.

FIG. 6 depicts the effects of refrigeration time and freezing parameters on cell viability for amniotic membrane.

FIG. 7 shows representative images of fresh amniotic (A and C) and chorionic (E) membranes as well as cryopreserved amniotic (B and D) and chorionic (F) membranes stained with live/dead stain. Live cells appear green, while dead cells appear red.

FIG. 8 depicts results from Mixed Lymphocyte Reaction (MLR) assay which measures expression of IL-2Ra from T-cells stimulated by various placental membranes from manufacturing intermediates—trophoblast, choriotrophoblast (CT), amnion with choriotrophoblast (ACT), amnion (AM), chorion (CM), and amnion with chorion (A/C).

FIG. 9 depicts results from MLR assay which measures expression of IL-2Ra from T-cells stimulated by various placental membranes from manufacturing intermediates and placental membranes after cryopreservation—Amnion (AM), Chorion (CM), Amnion with Chorion (A/C).

FIGS. 10A and B depict lipopolysaccharide (LPS) stimulated TNF-α released from various membrane preparations—Amnion+Chorion+Trophoblast (ACT), Chorion+Trophoblast (CT), Trophoblast (T), Amnion (AM), and Chorion (CM).

FIG. 11 shows expression of IL-2Rα from T-cells stimulated by choriotrophoblast (CT) which secreted high levels of TNF-α.

FIG. 12 shows images of cultured cells isolated from amniotic (A) and chorionic membranes (B), demonstrating plastic adherence. MSC isolated from human bone marrow aspirate are shown for comparison (C). Osteogenic potential of placental-derived cells is illustrated by purple stain for alkaline phosphatase (D).

FIG. 13 depicts expression of VEGF in fresh amniotic membrane as compared to cryopreserved amniotic membrane.

FIG. 14A depicts expression of IFN-2a and TGF-β3 in amniotic membrane homogenates. FIG. 14 B depicts expression of bFGF in amniotic membrane (AM) and chorionic membrane (CM) extracts.

FIG. 15 depicts expression of BMP-2, BMP-4, PLAB, PIGF (A), and IGF-1 (B) in amniotic membrane homogenates.

FIG. 16 depicts expression of EGF (A), IGFBP1 (B), and Adiponectin (C) in amniotic (AM), chorionic membranes (CM), and commercially available products.

FIG. 17 depicts the ratio of MMPs to TIMPs in amniotic, chorionic, and commercially available products.

FIG. 18 depicts expression of EGF in amniotic and chorionic membranes measured by ELISA in two separate placenta donors.

FIG. 19 shows that cryopreserved amniotic membranes produce high levels of anti-inflammatory cytokine PGE-2 when exposed to TNF-α.

FIG. 20 shows that cryopreserved amniotic membranes inhibit release of soluble pro-inflammatory cytokines such as IL-1α (A) and TNF-α (B) and upregulate the release of anti-inflammatory IL-10 (C) when co-cultured with activated immune cells.

FIG. 21 shows that amniotic membrane products exhibit a statistically significant (*p<0.05) ability to inhibit MMP activity as seen by the reduction in purple dye released.

FIG. 22 depicts inhibition of elastase by cryopreserved amniotic membranes.

FIG. 23 shows antioxidant capacity of cryopreserved amniotic membrane and ascorbic acid (a potent antioxidant).

FIG. 24 shows that cryopreserved amniotic membranes are able to rescue early-stage apoptotic Human Dermal Fibroblasts (HDFs). Apoptotic cells appear as bright blue dots due to their condensation of chromatin and nuclear fragmentations.

FIG. 25 shows that cryopreserved amniotic membranes promote Human Umbilical Vein Endothelial Cells (HUVECs) to form tubes and the number of formed tubes is significantly greater than negative control.

FIG. 26 depicts the Cell Biolabs 24-well Cytoselect wound healing assay.

FIG. 27 depicts representative images of Human Dermal Microvascular Endothelial Cells (HMVECs) treated with 5% conditioned media from amniotic, chorionic, or a combination of amniotic/chorionic preparations.

FIG. 28 depicts the promotion of endothelial cell migration by cryopreserved amniotic membrane (Amnion).

FIG. 29 depicts the promotion of fibroblast cell migration by cryopreserved amniotic membrane (Amnion).

FIG. 30 depicts the promotion of diseased keratinocyte migration by cryopreserved amniotic membrane (Amnion).

FIG. 31A-E depicts the remarkable efficacy of placental products for treating diabetic foot ulcers in patient 1.

FIG. 32A-D depicts the remarkable efficacy of placental products for treating diabetic foot ulcers in patient 2.

FIG. 33 depicts results of clinical study showing complete wound closure was significantly higher in patients that received placental product (31 of 50 patients, 62.0% wound closure) compared to control (10 of 47 patients, 21.0% wound closure, p=0.0001).

FIG. 34 depicts a Kaplan-Meier analysis showing a statistically greater probability of complete wound healing during the 12 week evaluation period for placental product compared to control (p<0.0001).

FIG. 35 shows that patients undergoing treatment with the placental product required statistically fewer applications in order to achieve complete wound closure, relative to the control arm (p=0.0001).

FIG. 36 depicts a Kaplan-Meier analysis showing patients in the control that cross-over to receive up to 12 weeks of placental product therapy (n=26) had a probability of wound closure of 67.8%.

FIG. 37A-F depicts a general overview of a method for surgical repair of a tendon that incorporates a cryopreserved membrane disclosed herein.

FIG. 38A-C depicts a general overview of a method for surgical repair of tendinosis that incorporates a cryopreserved membrane disclosed herein.

### DETAILED DESCRIPTION OF THE INVENTION

As used herein, the following definitions apply:

“Exemplary” (or “e.g.” or “by example”) means a non-limiting example.

“Chorionic tissue” or “Chorionic membrane” means the chorion or a portion thereof from placental tissue, e.g. the trophoblast, the somatic mesoderm, or combinations thereof.

“Amniotic tissue” or “Amniotic membrane” means the amnion or a portion thereof from placental tissue, e.g., the epithelium layer; the basement membrane; the compact layer; the fibroblast layer; and the intermediate (spongy) layer.

“Placental products” or “placental membrane” means the instant placental products and membranes disclosed and claimed herein. The term includes, and can be used interchangeably with, terms including placental membrane, cryopreserved placental membrane, cryopreserved chorioamniotic membrane, cryopreserved chorionic membrane, and cryopreserved amniotic membrane. Placental products may be used for tissue regeneration and wound repair.

Membranes and placental products that are “depleted of immunogenicity,” “depleted of immunogenic cells” or “depleted of immunogenic factors” or membranes and placental products that contain “depleted amounts of functional immunogenic cells” or “depleted amounts of one or more types of functional immunogenic cells” generally refer to a one or more placental product of the present technology that retains live therapeutic cells and/or retains therapeutic efficacy for the treatment of tissue injury (or defect) yet is free, substantially free, or depleted of at least one immunogenic cell type (e.g. CD14+ macrophages, trophoblasts, and/or maternal blood cells) and/or immunogenic factor that are otherwise present in a native placenta, amniotic membrane, or chorionic membrane. A membrane (like those of the presently described technology) that is free, substantially free, or depleted of immunogenic cell types and/or immunogenic factors includes membranes that may retain some amount of immunogenic cells/factors but the retained amount is at a level that is insufficient to produce a functional response (e.g., below detectable amounts, in negligible amounts, in amounts insufficient to produce a functional immune response).

“Extracellular matrix” or “ECM” as used herein refers to any one or more components of extracellular matrix that is associated with a tissue such as, for example, placental tissues including amniotic membrane, chorionic membrane, and/or chorioamniotic membrane. The term can include structural components of the ECM, such as collagens, laminins, fibronectin, hyaluronan, dermatan sulfate, heparin sulfate, chondroitin sulfate, decorin, and elastin, as well as soluble/functional therapeutic factors that may be present in the ECM (e.g., including proteins and fragments thereof).

“MSC” means mesenchymal stem cells and include fetal, neonatal, adult, or post-natal. “MSCs” include amniotic MSCs (AMSCs) and chorionic MSCs (CMSCs). MSCs generally express one or more of CD73, CD70, CD90, CD105, and CD166; and generally do not express CD45 and CD34. MSCs differentiate into mesodermal lineages (osteogenic, chondrogenic, and adipogenic).

“Native cells” or “tissue cells” means cells that are native, resident, or endogenous to the placental membrane, i.e. cells that are not exogenously added to the placental membrane, including amniotic and chorionic membranes.

“Native factors” means placental membrane factors that are native, resident, or endogenous to the placental membrane, i.e. factors that are not exogenously added to the placental membrane.

“Therapeutic cells,” or “beneficial cells” include cells and components present in the stromal layer and/or the epithelial layer of the placental membrane, and include, for example, MSCs, fibroblasts, and/or epithelial cells.

“Therapeutic factors” means placenta-, chorionic membrane-, or amniotic membrane-derived factors that promote wound healing. Therapeutic factors also encompass molecules that may be classified as cell growth factors/proteins, tissue repair factors/proteins, as well as other factors and proteins that generally promote wound healing. Non-limiting examples of therapeutic factors include antimicrobial factors, chemoattractants, remodeling proteins such as proteases and protease inhibitors, immunoregulatory factors, chemokines, cytokines, growth factors and other factors. Therapeutic factors also include factors that promote angiogenesis, cell proliferation and epithelialization. Non-limiting examples of such factors include TGFα, TGFβ1, TGFβ2, TGFβ3, EGF, HB-EGF, VEGF, VEGF-C, VEGF-D, HGF, PDGF-AA, PDGF-AB, PDGF-BB, PLGF, PEDF, Ang-2, IGF, IGFBP1, IGFBP2, IGFBP3, adiponectin, α2-macroglobulin, FGFs (e.g., FGF-2/bFGF, KGF, KDG/FGF-7), matrix metalloproteinases (e.g., MMP-1, MMP-2, MMP-3, MMP-7, MMP-8, MMP-9, MMP-10, MMP-13), tissue inhibitors of metalloproteinases (e.g., TIMP1, TIMP2), thrombospodins (e.g., TSP1, TSP2), fibronectin, IL-1RA, NGAL, defensin, G-CSF, LIF, IFN2α, PLAB, and SDF1b. The term “therapeutic factor” may be used interchangeably with the term “placental factor.”

“Stromal cells” refers to a mixed population of cells present (optionally in native proportions) composed of neonatal mesenchymal stem cells and neonatal fibroblasts. Both neonatal mesenchymal stem cells and neonatal fibroblasts are immunoprivileged; neither express surface proteins present on immunogenic cell types that trigger an immune response.

“Stromal layer” refers to the layers in the placental membrane that do not contain the epithelial layer.

In vitro describes the experiments and/or procedures performed outside of the living organism (e.g. under tissue culture conditions using artificial culture medium), including, but not limited to, culture expansion of cells.

In vivo describes experiments and/or procedures performed within an organism, for example, an animal or human.

A “cryopreservation agent” or “cryopreservative” or “cryoprotectant” are used interchangeably herein and are substances that help to prevent damage (e.g., cellular damage) during the freezing process. Suitable cryopreservation agents include, but are not limited to, Dimethyl Sulfoxide (DMSO), a glycerol, a glycol, a propylene glycol, an ethylene glycol, propanediol, polyethylene glycol (PEG), 1,2-propanediol (PROH) or a combination thereof. Other cryopreservation agents include, for example, one or more non-cell permeating cryopreservatives selected from, for example, polyvinyl pyrrolidione, a hydroxyethyl starch, a polysaccharide, a monosaccharide, an alginate, trehalose, raffinose, dextran, human serum albumin, ficoll, lipoproteins, polyvinyl pyrrolidone, hydroxyethyl starch, autologous plasma or a combination thereof. Other examples of useful cryopreservatives are described in Cryopreservation (BioFiles, Volume 5, Number 4 Sigma-Aldrich® Datasheet).

A “cryopreservation solution” or “cryopreservation media” refers to a composition comprising at least one cryopreservation agent. A cryopreservation solution or media may contain further components, for example, serum albumin, pharmaceutically acceptable carriers, buffers, electrolyte solutions, or saline (e.g., phosphate buffer saline). The cryopreservation solution or media may be a solution, a slurry, suspension, etc.

In a general sense the technology described herein provides for placental products comprising manipulated placental tissues. For example, the placental products can include cryopreserved amniotic membranes, cryopreserved chorionic membranes, and/or cryopreserved chorioamniotic membranes. In certain aspects the cryopreservation methods retain high amounts of viable placental cells (i.e., cells that are native to the placental tissue(s)) and provide for the depletion of immunogenic cells and factors associated with immunogenic cells. As such, the disclosure relates to placental products, and particularly membranes comprising cryopreserved amniotic, chorionic, and/or chorioamniotic membranes that comprise a combination of viable cells, therapeutic factors, extracellular matrix, and reduced immunogenicity, which find use in any number of beneficial therapeutic methods. In particular aspects discussed below, the membranes can be applied to a wound or a tissue defect, and provide amounts of viable cells, therapeutic factors, extracellular matrix that can directly or indirectly induce a change in the region to which the membrane is applied (e.g., an adaptive medicine). For example, the membranes can provide for improved healing of wounds, such as chronic wounds by providing viable cells, therapeutic factors, and extracellular matrix in amounts that can provide or promote the normal stages of wound healing by any of or more of promoting: (i) a reduction of the amount and/or activity of pro-inflammatory cytokines; (ii) an increase in the amount and/or activity of anti-inflammatory cytokines; (iii) a reduction of the amount and/or activity of reactive oxygen species; (iv) an increase in the amount and/or activity of antioxidant agents; (v) a reduction of the amount and/or activity of proteases; (vi) an increase in cell proliferation; (vii) an increase in angiogenesis; and/or (viii) an increase in cell migration. As a chronic wound environment can include any one or more of 1) high levels of proinflammatory cytokines, 2) low levels of anti-inflammatory cytokines, 3) high levels of proteases and low levels of their inhibitors, as well as 4) high levels of oxidants and low levels of antioxidant to counter balance, the characteristics and functionality of the cryopreserved membranes disclosed herein are well suited to such applications.

In some embodiments, the present technology discloses placental products for clinical use, including use in wound healing such as diabetic foot ulcers, venous leg ulcers, and burns. The manufacturing process optionally eliminates essentially all potentially immunogenic cells from the placental membrane while preserving specific cells that play an important role in wound healing.

In some embodiments, the present technology discloses a placental product that is selectively devitalized. In embodiments, the placental product may be selectively depleted of substantially all immunogenic cells. In some embodiments, the membranes do not contain culture expanded cells.

In some embodiments, the present technology provides a placental product that comprises at least one therapeutic factor, or a combination of any two or more therapeutic factors that are disclosed herein or otherwise known such as, for example, Insulin-like Growth Factor Binding Protein 1 (IGFBP1) or adiponectin. In embodiments, the placental product may comprise Epidermal Growth Factor (EGF) and/or IGFBP1. In embodiments, the disclosure provides a placental product that can be used to increase the anti-inflammatory activity. In embodiments, the disclosure provides a placental product that can be used to increase the antioxidant activity. In embodiments, the disclosure provides a placental product that can be used to reduce adhesion and fibrosis and generally promote anti-scarring activity. In some embodiments, the disclosure provides a placental product that can be used to increase cell migration. In some embodiments, the disclosure provides a placental product that can be used to increase the formation of vasculature.

In some embodiments, the present technology discloses a method of cryopreserving a placental product that preserves the viability of specific beneficial cells that are a primary source of factors for the promotion of wound healing while selectively depleting immunogenic cells (e.g. killing or rendering non-immunogenic).

In some embodiments, the present technology discloses a bioassay to test immunogenicity of manufactured placental products.

In some embodiments, the present technology discloses a placental product exhibiting a therapeutic ratio of MMP to TIMP comparable to that exhibited in vivo. The present inventors have identified a need for the development of placental products exhibiting a ratio of MMP-9 and TIMP1 of about 7-10 to one.

In some embodiments, the present technology discloses a placental product that comprises α2-macroglobulin.

In some embodiments, there is now provided a placental product that is capable of inactivating serine proteases, cysteine proteases, aspartic proteases, and metalloproteases. In other embodiments, there is now provided a placental product that inactivates serine proteases. In other embodiments, there is now provided a placental product that inactivates cysteine proteases. In aspects, there is now provided a placental product that inactivates aspartic proteases. In other aspects, there is now provided a placental product that inactivates metalloproteases.

In some embodiments, the present technology discloses a placental product that comprises bFGF.

In some embodiments, the present technology discloses a placental product exhibiting a MMP to TIMP ratio favorable for wound healing.

In some embodiments, the present technology discloses a cell migration assay capable of evaluating the wound-healing potential of a placental product.

IGFBP1 and adiponectin are among the factors contemplated herein that are important for wound healing. Evaluation of proteins derived from placental products prepared according to the presently disclosed technology reveal that EGF is one of the major factors secreted in higher quantities by these tissues. Additionally, the importance of EGF for wound healing together with high levels of EGF detected in the presently disclosed amniotic membranes support selection of EGF as a potency marker for evaluation of membrane products manufactured for clinical use pursuant to the present disclosure.

The present technology discloses a cryopreservation procedure for a placental product that selectively depletes immunogenic cells and preserves the viability of other beneficial cells (including at least one or more of mesenchymal stem cells, epithelial cells and fibroblasts). In some embodiments, the beneficial cells are the primary source of factors for the promotion of healing.

Placental products, their usefulness, and their immunocompatability are surprisingly enhanced by depletion of maternal trophoblast and selective elimination of CD14+ fetal macrophages. Immunocompatability can be demonstrated by any means commonly known by the skilled artisan, such demonstration can be performed by the mixed Lymphocyte Reaction (MLR) and by lipopolysaccharide (LPS)-induced Tumor Necrosis Factor (TNF)-α secretion.

The instant placental products contain basic Fibroblast Growth Factor (bFGF) optionally at a substantial concentration.

The instant placental products provide and/or optionally secrete factors that stimulate cell migration and/or wound healing. The presence of such factors can be demonstrated by any commonly recognized method.

For example, commercially available wound healing assays exist (Cell Biolabs) and cell migration can be assessed by using Human Dermal Microvascular Endothelial Cells (HMVEC-d) (Lonza Inc.). In one embodiment, conditioned medium from the present placental products enhance cell migration.

The placental products disclosed herein are useful in treating a number of wounds including: tendon repair, cartilage repair (e.g. femoral condyle, tibial plateau), ACL replacement at the tunnel/bone interface, dental tissue augmentation, fistulas (e.g. Crohn's disease, G-tube, tracheoesophogeal), missing tissue at adhesion barriers (e.g. nasal septum repair, vaginal wall repair, abdominal wall repair, tumor resection), dermal wounds (e.g. partial thickness burns, toxic epidermal necrolysis, epidermolysis bullosa, pyoderma gangrenosum, ulcers e.g. diabetic ulcers (e.g. foot) and venous leg ulcers), surgical wounds, hernia repair, tendon repair, bladder repair, periosteum replacement, keloids, organ lacerations, epithelial defects, and repair or replacement of a tympanic membrane.

The placental products disclosed herein exhibit one or more of the following properties beneficial to the wound healing process:
a. an epithelial cell layer, wherein the approximate number of cells per cm2 of the amniotic membrane is about 500 to about 360,000; about 10,000 to about 360,000; about 10,000 to about 200,000; or about 20,000; about 25,000; about 30,000; about 35,000; about 40,000; about 45,000; about 50,000; about 55,000; about 60,000; about 65,000; about 70,000; about 75,000; about 80,000; about 85,000; about 90,000; about 95,000; about 100,000; about 105,000; about 110,000; about 115,000; about 120,000; about 125,000; about 130,000; about 135,000; about 140,000; about 145,000; about 150,000; about 155,000; about 160,000; about 165,000; about 170,000; about 175,000; about 180,000; about 185,000; about 190,000; or about 195,000.
b. a thick basement membrane (comprising one or more of Collagen Type I, III, IV, laminin, and fibronectin),
c. a stromal layer;
d. an amniotic membrane with a thickness of about 20 to about 500 μm,
e. high thrombin activity,
f. low immunogenicity,
g. cryopreserved/cryopreserveable,
h. amniotic MSCs,
i. analgesic effect
j. reduces scarring,
k. reduces pro-inflammatory proteins such as IL-1α and TNF-α,
l. increases anti-inflammatory proteins such as IL-10,
m. immunomodulation for example by inhibition of CD8+ and CD4+ lymphocyte proliferation and/or increased amount and/or activity of CD4+ Treg cells,
n. antibacterial proteins such as defensins and allantoin (bacteriolytic proteins),
o. angiogenic and mitogenic factors that promote blood vessel formation and re-epithelialization such as EGF, HGF, and VEGF,
p. cells that are positive for CD70, CD73, CD90, CD105, and CD166 and negative for CD45 and CD34,
q. cells that express HLA-G,
r. cells that express IDO and FAS ligand, which likely contribute to immune tolerance,
s. cells with a capacity to differentiate into Human Amniotic Epithelial Cells (hAECs)
t. cells with a capacity to differentiate to neural, hepatocyte, and pancreatic cells,
u. Human Amniotic Mesenchymal Stem Cells (hAMSCs) with the capacity to differentiate to mesodermal lineages (osteogenic, chondrogenic, and adipogenic) and to all three germ layers-ectoderm (neural), mesoderm (skeletal muscle, cardiomyocytic, and endothelial), and ectoderm (pancreatic),
v. hAMSCs express CD49d and this distinguishes them from hAECs,
w. hAMSCs that positive for the embryonic cytoplasmic marker Oct-4 that plays a role in maintaining stemness, or multipotency and self-renewal,
x. hAECs that are positive for SSEA-3, SSEA-4, TRA-1-60, TRA-1-81, and negative for SSEA-4 and non-tumorigenic.

The present inventors have now identified a need for the development of placental products that do not contain culture expanded cells.

The present inventors have now identified a need for the development of placental products comprising IGFBP1.

The present inventors have now identified a need for the development of placental products comprising adiponectin.

The present inventors have now identified a need for the development of placental products exhibiting a protease-to-protease inhibitor ratio favorable for wound healing.

The present inventors have now identified a need for the development of a method of cryopreserving placental products that preserves the viability of beneficial cells that are a primary source of factors for the promotion of the wound healing process while selectively depleting immunogenic cells from membranes.

The present inventors have now identified a need for the development of a bioassay to test immunogenicity of manufactured placental products.

The present inventors have now identified a need for the development of placental products exhibiting a ratio of MMP to TIMP comparable to that exhibited in vivo.

The present inventors have now identified a need for the development of placental products exhibiting a ratio of MMP-9 and TIMP1 of about 7-10 to one.

The present inventors have now identified a need for the development of placental products that comprise α2-macroglobulin.

The present inventors have now identified a need for the development of placental products that inactivate serine proteases, cysteine proteases, aspartic proteases, and metalloproteases. The present inventors have now identified a need for the development of placental products that inactivate serine proteases. The present inventors have now identified a need for the development of placental products that inactivate cysteine proteases. The present inventors have now identified a need for the development of placental products that inactivate aspartic proteases. The present inventors have now identified a need for the development of placental products that inactivate metalloproteases.

The present inventors have now identified a need for the development of placental products that comprise bFGF.

The present inventors have now identified a need for the development of a cell migration assay to evaluate the potential of placental membrane products.

The present inventors have now identified a need for the development of a placental product for wound healing that comprises mesenchymal stem cells, epithelial cells and fibroblasts.

### Placental Product

### Overview

In one aspect, the disclosure provides a membrane comprising cryopreserved amniotic membrane, wherein after cryopreservation and subsequent thawing the amniotic membrane comprises:

A) tissue cells, wherein said tissue cells are native to the amniotic membrane and greater than 40% of said tissue cells are viable;

B) one or more therapeutic factors that are native to the amniotic membrane;

C) extracellular matrix that is native to the amniotic membrane; and

D) depleted amounts of one or more types of functional immunogenic cells.

In another aspect, the disclosure provides a membrane comprising cryopreserved amniotic membrane, wherein after cryopreservation and subsequent thawing the amniotic membrane comprises:

A) a stromal layer comprising viable cells, one or more therapeutic factors, and extracellular matrix,

B) an epithelial layer comprising viable cells and one or more therapeutic factors; and

C) depleted amounts of one or more types of functional immunogenic cells.

wherein greater than 40% of the combined cells in the stromal layer and epithelial layer are viable cells.

In another aspect, the disclosure provides a membrane comprising cryopreserved amniotic membrane having one or more tissue components, wherein after cryopreservation and subsequent thawing the one or more tissue components comprise:

A) tissue cells, wherein said tissue cells are native to the amniotic membrane and greater than 40% of said tissue cells are viable;

B) one or more therapeutic factors that are native to the amniotic membrane; and

C) extracellular matrix that is native to the amniotic membrane;

wherein the amniotic membrane has depleted amounts of types of functional immunogenic cells; and wherein the one or more tissue components is present in an amount effective to provide a therapeutic benefit.

In one aspect, the disclosure provides a membrane comprising cryopreserved amniotic membrane having one or more tissue components, wherein after cryopreservation and subsequent thawing the one or more tissue components comprise:

A) tissue cells, wherein said tissue cells are native to the amniotic membrane and greater than 40% of said tissue cells are viable;

B) one or more therapeutic factors that are native to the amniotic membrane; and

C) extracellular matrix that is native to the amniotic membrane;

wherein the amniotic membrane has depleted amounts of types of functional immunogenic cells, and wherein the one or more tissue components is present in an amount effective to:

(ii) reduce the amount and/or activity of pro-inflammatory cytokines;

(ii) increase the amount and/or activity of anti-inflammatory cytokines;

(iii) reduce the amount and/or activity of reactive oxygen species;

(iv) increase the amount and/or activity of antioxidant agents;

(v) reduce the amount and/or activity of proteases;

(vi) increase cell proliferation;

(vii) increase angiogenesis; and/or

(viii) increase cell migration.

In another aspect, the disclosure provides a membrane comprising cryopreserved placental membrane, wherein after cryopreservation and subsequent thawing the placental membrane comprises:

A) tissue cells, wherein said tissue cells are native to the placental membrane and greater than 40% of said tissue cells are viable;

B) one or more therapeutic factors that are native to the placental membrane;

C) extracellular matrix that is native to the placental membrane; and

D) depleted amounts of one or more types of functional immunogenic cells.

In some embodiments of the aspect comprising a cryopreserved placental membrane, the placental membrane comprises amniotic membrane and chorionic membrane.

In some embodiments of the above aspects, the depleted amounts of functional immunogenic cells produce immunogenic factors in amounts that are below levels sufficient to produce an immune response. In some embodiments, the depleted amounts of functional immunogenic cells produce immunogenic factors in amounts below detectable limits. As described herein, the depleted amounts of functional immunogenic cells may be selected from any one or more of maternal blood cells, neonatal blood cells, cord blood cells, tissue macrophages, and trophoblasts. In further embodiments, the depleted amounts of functional immunogenic cells comprise one or more tissue macrophage. The tissue macrophage can be of any characterized type of macrophage such as, for example, tissue macrophages selected from the group consisting of CD11b+, CD14+, CD18+, CD40+, and CD86+ and/or combinations thereof. In further embodiments, the depleted amounts of functional immunogenic cells produce one or more immunogenic factors (e.g., TNF-α, and/or other immunogenic factors described herein or otherwise known) in amounts that are below levels sufficient to produce an immune response. In further embodiments, the immunogenic factors may be produced in amounts that are negligible or below detectable limits. In some embodiments, less than 10% of viable cells are functional immunogenic cells (e.g., less that 10%, 9%, 8%, 7%, 6%, 5%, 4%, 3%, 2%, or less that 1% of viable cells).

In embodiments of the above aspects, the membrane comprises tissue cells wherein about 50% to about 100% of said tissue cells are viable. In some embodiments, about 60% to about 100% of said tissue cells are viable. In further embodiments, about 70% to about 100% of said tissue cells are viable. In further embodiments the viable cells may comprise mesenchymal stems cells (MSCs), fibroblasts, and/or epithelial cells.

In various embodiments of the above aspects, the membranes provide one or more tissue components (e.g., viable cells, one or more therapeutic factors, and/or extracellular matrix) in an amount that is effective to promote any of the activities of (i)-(viii) in vitro or in vivo.

In vitro describes the experiments and/or procedures performed outside of the living organism (e.g. under tissue culture conditions using artificial culture medium). In vivo describes experiments and/or procedures performed within an organism, for example, an animal or human.

Various embodiments of the above-described aspects may further comprise a delivery substrate, such that the membrane is fixed to the delivery substrate.

In embodiments of the above aspects, the membrane may be stored for an extended period of time prior to subsequent thawing. In some embodiments the extended period of time is from about 6 months to at least about 36 months or more, alternatively from about 6 months to at least about 24 months or greater, alternatively from about 6 months to about 25 months or greater, alternatively from about 6 months to at least about at least about 12 months or greater, alternatively from about 6 months to about 10 months, alternatively from about 6 months, alternatively from about 3 months to about 6 months, alternatively from about 1 month to about 3 months, including other monthly and day derivations thereof for the various time periods described herein. In these embodiments, the viability of the tissue cells is substantially maintained upon thawing. In some embodiments, the viability of the tissue cells is substantially maintained for at least 24 months when stored frozen.

In embodiments of the above aspects, the membrane can be thawed and ready for use within 30 minutes of the start of a thawing method, such as described herein or as modified from generally known methods.

In some embodiments of the above aspects, the membrane can be stored in saline up to an hour after thawing and still maintain about 70% viable cells.

One embodiment of the present technology provides a placental product comprising a cryopreservation solution and an amniotic membrane, wherein the amniotic membrane comprises viable native therapeutic cells and native therapeutic factors, and wherein the cryopreservation solution comprises an amount of a cryopreservative that is effective to provide for a cryopreserved product. According to this embodiment, the amniotic membrane is substantially free of at least one at least one or both of the following immunogenic cell types: CD14+ macrophages and maternal blood cells.

In one embodiment, the amniotic membrane comprises one or more layers which exhibit the architecture of the native amniotic membrane (e.g. has not been homogenized or treated with collagenase).

In one embodiment, the placental membrane is suitable for dermal application to a wound.

With the teachings provided herein, the skilled artisan can now produce the present placental products or membranes. The present disclosure provides methods of manufacture that produce the technical features of the present placental products. Accordingly, in one embodiment, the placental product is manufactured by steps taught herein. The present placental products are not limited to products manufactured by the methods taught here. For example, products of the present technology could be produced through methods that rely on screening steps; e.g. steps to screen for preparations with the described technical features and superior properties.

The present placental product comprises one or more of the following technical features:
a. the viable therapeutic native cells are capable of differentiating into cells of more than one lineage (e.g. osteogenic, adipogenic and/or chonodrogenic lineages),
b. the native therapeutic factors include IGFBP1,
c. the native therapeutic factors include adiponectin,
d. the native therapeutic factors include α2-macroglobulin,
e. the native therapeutic factors include bFGF,
f. the native therapeutic factors include EGF,
g. the native therapeutic factors include particular amounts of MMP-9 and TIMP1,
h. the native therapeutic factors include MMP-9 and TIMP1 in a ratio of about 7 to about 10,
i. the placental product does not comprise culture expanded cells,
j. the cryopreservative solution may be present in an amount of greater than about 15 mL,
k. the cryopreservative comprises DMSO,
l. the cryopreservation solution further comprises albumin, optionally wherein the albumin is Human Serum Albumin (HSA),
m. the cryopreservative comprises DMSO and albumin (e.g. HSA),
n. comprises about 5,000 to about 500,000 cells/cm², about 5,000 to about 240,000 cells/cm² or about 20,000 to about 60,000 cells/cm²,
o. the amniotic membrane comprises at least: about 2,000, or about 2,400, or about 4,000 or about 6,000, or about 8,000, or about 10,000, or about 10,585, or about 15,000 stromal cells per unit cm² of the amniotic membrane,
p. the amniotic membrane comprises about 2,000 to about 15,000 of stromal cells per cm² of the amniotic membrane,
q. comprises stromal cells wherein about 40%, or about 50%, or about 60%, or about 70%, or about 74.3%, or about 83.4% or about 90%, or about 92.5% or at least about 100% of the stromal cells are viable after a freeze-thaw cycle,
r. comprises stromal cells wherein about 40% to about 92.5% of the stromal cells are viable after a freeze-thaw cycle,
s. the amniotic membrane has a thickness of about 20 μm to about 500 μm; about 20 μm to about 200 μm; or about 20 μm to about 100 μm.
t. secretes less than about any of: 350 pg/cm², 225 pg/cm², 100 pg/cm² or 70 pg/cm² or less TNF-α into a tissue culture medium upon placing a 2 cm×2 cm piece of the tissue product in a tissue culture medium and exposing the tissue product to a bacterial lipopolysaccharide for about 20 to about 24 hours,
u. after refrigeration, cryopreservation and thawing, secretes less than about any of: 350 pg/cm², 225 pg/cm², 100 pg/cm² or 70 pg/cm² or less TNF-α into a tissue culture medium upon placing a 2 cm×2 cm piece of the tissue product in a tissue culture medium and exposing the tissue product to a bacterial lipopolysaccharide for about 20 to about 24 hours,
v. further comprises an chorionic membrane,
w. amniotic membrane comprises a layer of epithelial cells,
x. further comprises an chorionic membrane, wherein the amniotic membrane and the chorionic membrane are associated to one another in the native configuration,
y. further comprises an chorionic membrane, wherein the amniotic membrane and the chorionic membrane are not attached to one another in the native configuration,
z. further comprises a chorionic membrane wherein the chorionic membrane comprises about 2 to about 4 times more stromal cells relative to the amniotic membrane,
aa. does not comprise a chorionic membrane;
bb. comprises chorionic membrane, wherein the chorionic membrane comprises about 2 to about 4 times more stromal cells relative to the amniotic membrane, and
cc. is suitable for dermal application to a wound.

### Cells

According to the present technology, a placental product comprises native therapeutic cells of the amniotic membrane. The cells comprise one or more of MSCs, fibroblasts, and epithelial cells.

In one embodiment, the native therapeutic cells comprise viable MSCs.

In one embodiment, the native therapeutic cells comprise viable fibroblasts.

In one embodiment, the native therapeutic cells comprise viable epithelial cells.

In one embodiment, the native therapeutic cells comprise viable MSCs and viable fibroblasts.

In one embodiment, the native therapeutic cells comprise viable MSCs, viable fibroblasts, and viable epithelial cells.

In one embodiment, the therapeutic native cells are viable cells capable of differentiating into cells of more than one lineage (e.g. osteogenic, adipogenic and/or chonodrogenic lineages).

In one embodiment, the placental product comprises about 500 to about 360,000 cells per cm² or about 40,000 to about 90,000 cells per cm².

In one embodiment, the placental product comprises at least: about 2,000, or about 2,400, or about 4,000, or about 6,000, or about 8,000, or about 10,000, or about 10,585, or about 15,000, or about 180,000 stromal cells per unit cm² of the placental product.

In one embodiment, the placental product comprises about 2,000 to about 15,000 of stromal cells per cm² of the placental product.

In one embodiment, the placental product comprises stromal cells wherein at least: about 40%, or about 50%, or about 60%, or about 70%, or about 74.3%, or about 83.4% or about 90%, or about 92.5%, or about 100% of the stromal cells are viable after a freeze-thaw cycle.

In one embodiment, the placental product comprises stromal cells wherein about 40% to about 100% of the stromal cells are viable after a freeze-thaw cycle, alternatively from about 40% to about 99.9% of the stromal cells are viable, alternatively wherein about 70% to about 99% of the stromal cells are viable.

In one embodiment, the placental product comprises less than about 1% of CD14+ macrophages per total cells.

In one embodiment, the amniotic membrane of the placental product comprises about 2 to about 4 times less stromal cells relative to a chorionic membrane derived from the same placenta.

In one embodiment, the placental product further comprises chorionic membrane containing about 2 to about 4 times more stromal cells relative to the amniotic membrane.

In one embodiment, the amniotic membrane of the placental product comprises MSCs in an amount of up to about 70% of the total number of cells in the amniotic membrane product. In some embodiments the product comprises MSCs in an amount of: at least about 1%, at least about 2%, at least about 3%, at least about 4%, at least about 5%, about 1% to about 10%, or about 3% to about 100%, relative to the total number of cells in the amniotic membrane of the placental product. In some embodiments, of the total number of MSCs in the amniotic membrane product, at least: about 40%, about 50%, about 60%, about 70%, or about 100% of the MSCs are viable after a freeze-thaw cycle.

In one embodiment, the amniotic membrane of the placental product comprises fibroblasts in an amount of up to about 100% of the total number of cells in the amniotic membrane product. In some embodiments, the product comprises fibroblasts in an amount of: about 1%, about 20%, about 5% to about 15%, at least about 1%, at least about 2%, at least about 3%, or at least about 4% relative to the total number of cells in the amniotic membrane of the placental product. In some embodiments, of the total number of fibroblasts in the amniotic membrane product, at least: about 40%, about 50%, about 60%, about 70%, or about 100% of the fibroblasts are viable after a freeze-thaw cycle.

In one embodiment, the amniotic membrane of the placental product comprises stromal cells in an amount of: about 5% to about 100%, about 5% to about 50%, about 5% to about 30%, about 10% to about 30%, about 15% to about 25%, at least about 5%, at least about 10%, or at least about 15%, relative to the total number of cells in the amniotic membrane of the placental product. Optionally, at least: about 40%, about 50%, about 60%, about 70%, or about 100% of the stromal cells are viable after a freeze-thaw cycle.

In one embodiment, the amniotic membrane of the placental product comprises epithelial cells in an amount of: about 60% to about 100%, about 60% to about 90%, about 70% to about 90%, about 40% to about 90%, about 50% to about 90%, at least about 40%, at least about 50%, least about 60%, or at least about 70%, relative to the total number of cells in the amniotic membrane of the placental product. Optionally, at least: about 40%, about 50%, about 60%, about 70%, or about 100% of the epithelial cells are viable after a freeze-thaw cycle.

In embodiments, the total number of functional macrophages may comprise less than about 10% of viable cells (e.g., less than about 10%, about 9%, about 8%, about 7%, about 6%, about 5%, about 4%, about 3%, about 2%, or less than about 1%.

In one embodiment, the amniotic membrane of the placental product comprises fibroblasts and MSCs in a ratio of: about 4:1 to about 1:1 or about 3:1 to about 3:2, or about 2:1.

In one embodiment, the amniotic membrane of the placental product comprises MSCs in an amount of: at least about 1,000 cells/cm², at least about 2,000 cells/cm², about 1,000 to about 5,000 cells/cm², or about 2,000 to about 5,000 cells/cm². Optionally, at least: about 40%, about 50%, about 60%, about 70%, or about 100% of the MSCs are viable after a freeze-thaw cycle.

In one embodiment, the amniotic membrane of the placental product comprises fibroblasts in an amount of: at least about 2,000 cells/cm², at least about 4,000 cells/cm², about 2,000 to about 9,000 cells/cm², or about 2,000 to about 9,000 cells/cm². Optionally, at least: about 40%, about 50%, about 60%,about 70%, or about 100% of the fibroblasts are viable after a freeze-thaw cycle.

In one embodiment, the amniotic membrane of the placental product comprises stromal cells in an amount of: at least about 4,000, at least about 8,000 cells/cm², about 4,000 to about 18,000 cells/cm², or about 4,000 to about 18,000 cells/cm². Optionally, at least: about 40%, about 50%, about 60%, about 70%, or about 100% of the stromal cells are viable after a freeze-thaw cycle.

In one embodiment, the amniotic membrane of the placental product comprises epithelial cells in an amount of: at least about 10,000 cells/cm², at least about 20,000 cells/cm², at least about 32,000 cells/cm², about 10,000 to about 72,000 cells/cm², about 20,000 to about 72,000 cells/cm², or about 32,000 to about 72,000 cells/cm² Optionally, at least: about 40%, about 50%, about 60%, about 70%, or about 100% of the epithelial cells are viable after a freeze-thaw cycle.

In one embodiment, the amniotic membrane of the placental product comprises functional macrophages in an amount of: less than about 3,000 cells/cm², less than about 1,000 cells/cm², or less than about 500 cells/cm².

In one embodiment, the placental product comprises a layer of epithelial cells.

In one embodiment, the placental product comprises a chorionic membrane but is substantially free of trophoblasts.

In one embodiment, the placental product is substantially free of functional CD14+ macrophages.

In one embodiment, the placental product is substantially free of maternal blood cells.

In one embodiment, the placental product is substantially free of trophoblasts, functional CD14+ macrophages, and maternal blood cells. Optionally, the placental product comprises viable stromal cells. Optionally, the placental product comprises viable MSCs. Optionally, the placental product comprises viable fibroblasts. Optionally, the placental product comprises viable epithelial cells. Optionally, the placental product comprises viable MSCs, fibroblasts, and epithelial cells.

In one embodiment, the placental product comprises a chorionic membrane but is substantially free of maternal blood cells.

In one embodiment, the placental product comprises a chorionic membrane but is substantially free of maternal blood cells and/or trophoblast cells.

In one embodiment, the placental product is substantially free of culture expanded cells.

### Placental Factors

According to the present technology, a placental product comprises native therapeutic factors of the amniotic membrane, such as defined above and otherwise described herein in various aspects and embodiments, including the Examples. Table 10 provides a list of therapeutic factors tested and their functions.

In one embodiment, the factors include one or more of: IGFBP1, adiponectin, α2-macroglobulin, bFGF, EGF, MMP-9, and TIMP1. Optionally, the factors are present in amounts/cm² that are substantially similar to that of a native amniotic membrane or layer thereof (e.g. ±10% or 20%).

In one embodiment, the factors include VEGF, PDFG, HGF, SDF-1, KGF, IGFBP1, adiponectin, α2-macroglobulin, bFGF, EGF, MMP-9, and TIMP1, and the like. Optionally, the factors are present in ratios that are substantially similar to that of a native amniotic membrane thereof. Optionally, the factors are present in amounts/cm² that are substantially similar to that of a native amniotic membrane or layer thereof (e.g. ±10% or 20%).

In one embodiment, the factors include MMP-9 and TIMP1 in a ratio of about 7 to about 10 (e.g. about 7). Optionally, the factors are present in amounts/cm² that are substantially similar to that of a native amniotic membrane or layer thereof (e.g. ±10% or 20%).

In one embodiment, the factors include one or more (e.g. a majority or all) of the factors listed in Table 10. Optionally, the factors are present in ratios that are substantially similar to that of a native amniotic membrane or layer thereof. Optionally, the factors are present in amounts/cm² that are substantially similar to that of a native amniotic membrane or layer thereof (e.g. ±10% or 20%).

In one embodiment, the placental product thereof comprises substantially less TNF-α/cm² than a native amniotic membrane or layer thereof, respectively.

In one embodiment, the placental product thereof secretes substantially less TNF-α/cm² than a native placental product or layer thereof, respectively.

In one embodiment, the placental product secretes less than about any of: 420 pg/mL, 350 pg/mL, or 280 pg/mL TNF-α into a tissue culture medium upon placing a 2 cm×2 cm piece of the tissue product in a tissue culture medium and exposing the tissue product to a bacterial lipopolysaccharide for about 20 to about 24 hours.

In one embodiment, after refrigeration, cryopreservation and thawing, the placental product secretes less than about any of: 420 pg/mL, 350 pg/mL, or 280 pg/mL TNF-α into a tissue culture medium upon placing a 2 cm×2 cm piece of the tissue product in a tissue culture medium and exposing the tissue product to a bacterial lipopolysaccharide for about 20 to about 24 hours.

In one embodiment, the placental product further comprises an exogenously added inhibitor of TNF-α. Optionally, the inhibitor of TNF-α is PGE2.

In one embodiment, the product may be treated with an antibiotic or an antibiotic cocktail. One non-limiting example of an antibiotic cocktail includes gentamicin sulfate, vancomycin HCl, and amphotericin B.

### Architecture

A placental product of the present technology comprises one or more layers which exhibit the architecture of the native amniotic membrane. With the teachings provided herein, the skilled artisan will recognize placental layers that exhibit native architecture, for example, layers that have not been homogenized or treated with collagenase or other enzyme that substantially disrupts the layer.

In one embodiment, the placental product comprises a stromal layer with native architecture of the amniotic membrane.

In one embodiment, the placental product comprises a basement membrane with native architecture of the amniotic membrane.

In another embodiment, the placental product comprises an epithelial layer with native architecture of the amniotic membrane.

In one embodiment, the placental product comprises a stromal layer and an epithelial cell layer with native architecture of the amniotic membrane.

In one embodiment, the placental product or amniotic membrane thereof has a thickness of about 20 μm to about 500 μm; about 20 μm to about 200 μm; or about 20 μm to about 100 μm.

In one embodiment, the placental product comprises a chorionic membrane but is substantially free of trophoblasts. In one embodiment, the placental product comprises a basement membrane with native architecture of the chorionic membrane and the chorionic membrane is substantially free of trophoblasts. Optionally, the maternal portion in contact with the chorionic membrane comprises fragments of extracellular matrix proteins. Optionally, the placental product has been treated with Dispase (e.g. Dispase II) and/or a substantial portion of the extracellular matrix protein fragments comprises terminal leucine or phenylalanine.

In one embodiment, the placental product further comprises a chorionic membrane. Optionally, the amniotic membrane and the chorionic membrane in the placental product are associated to one another in the native configuration. Alternatively, the amniotic membrane and the chorionic membrane are not attached to one another in the native configuration.

In one embodiment, the placental product does not comprise a chorionic membrane.

### Formulation

According to the present technology, the placental product can be formulated with a cryopreservation solution.

In one embodiment, the cryopreservation solution comprising one or more cell-permeating cryopreservatives, one or more non cell-permeating cryopreservatives, or a combination thereof.

Optionally, the cryopreservation solution comprises one or more cell-permeating cryopreservatives selected from DMSO, a glycerol, a glycol, a propylene glycol, an ethylene glycol, or a combination thereof.

Optionally, the cryopreservation solution comprises one or more non cell-permeating cryopreservatives selected from polyvinylpyrrolidone, a hydroxyethyl starch, a polysaccharide, a monosaccharides, a sugar alcohol, an alginate, a trehalose, a raffinose, a dextran, or a combination thereof.

Other examples of useful cryopreservatives are described in “Cryopreservation” (BioFiles Volume 5 Number 4—Sigma-Aldrich® datasheet).

In one embodiment, the cryopreservation solution comprises a cell-permeating cryopreservative, wherein the majority of the cell-permeating cryopreservative is DMSO. Optionally, the cryopreservation solution does not comprise a substantial amount of glycerol.

In one embodiment, the cryopreservation solution comprises DMSO. Optionally, the cryopreservation solution does not comprise glycerol in a majority amount. Optionally, the cryopreservation solution does not comprise a substantial amount of glycerol.

In one embodiment, the cryopreservation solution comprises additional components such as albumin (e.g. HSA or BSA), an electrolyte solution (e.g. Plasma-Lyte), or a combination thereof.

In one embodiment, the cryopreservation solution comprises 1% to about 15% albumin by volume and about 5% to about 20% cryopreservative by volume (e.g. about 10%). Optionally, the cryopreservative comprises DMSO. In some embodiments, the cryopreservation solution comprises about 5% to about 100% of the cryopreservative, alternatively about 5% to about 20%.

In one embodiment, the placental product is formulated in greater than about 20 mL or about 50 mL of cryopreservation solution. Optionally, the cryopreservative solution comprises at least one cryopreservative (or cryopreservative agent). In some aspects, the at least one cryopreservative comprises DMSO (e.g. if there is more than one cryopreservative, DMSO is found in a majority amount of total cryopreservative). Optionally, the cryopreservation solution does not comprise a substantial amount of glycerol.

In some embodiments, the composition comprises cryopreservation solution. The cryopreservation solution may be added to a container containing the placental product, optionally as membrane-mounted composition (e.g., on nitrocellulose). Preferably, a sufficient amount of cryopreservation solution is added to protect the membrane during the subsequent freezing steps. The infusion of the membrane with the cryopreservation solution maintains viability of the cells contained within the membrane. While suitable cryopreservation solutions are known in the art, in one embodiment, the cryopreservation comprises storage in a cryopreservation solution comprising one or more cell-permeating cryopreservatives, one or more non-cell permeating cryopreservatives, or a combination thereof.

Suitable cryopreservatives include, but are not limited to, DMSO, a glycerol, a glycol, a propylene glycol, an ethylene glycol, propanediol, polyethylene glycol (PEG), 1,2-propanediol (PROH) or a combination thereof. In some embodiments, the cryopreservation solution may contain one or more non-cell permeating cryopreservative selected from polyvinyl pyrrolidione, a hydroxyethyl starch, a polysaccharide, a monosaccharide, an alginate, trehalose, raffinose, dextran, human serum albumin, ficoll, lipoproteins, polyvinyl pyrrolidone, hydroxyethyl starch, autologous plasma or a combination thereof. Other examples of useful cryopreservatives are described in Cryopreservation (BioFiles, Volume 5, Number 4 Sigma-Aldrich® Datasheet).

For example, a suitable cryopreservation solution comprises a cryopreservative, in an amount of at least about 0.001% to 100%, suitably in an amount from about 2% to about 20%, preferably about 5% to about 10% by volume, for example DMSO. In some instances, the cryopreservation solution comprises at least about 2% cryopreservative (e.g. DMSO). Further, the cryopreservation solution may comprise serum albumin or other suitable proteins. In some embodiments, the cryopreservation solution comprises from about 1% to about 20% serum albumin or other suitable proteins, alternatively from about 1% to about 10%. Serum albumin or other suitable proteins are present to help stabilize the membrane during the freeze-thaw process and to reduce the damage to cells, maintaining viability. Serum albumin may be human serum albumin or bovine serum albumin. The cryopreservation solution may further comprise a physiological buffer or saline, for example, phosphate buffer saline.

During the cryopreservation process, a container may be filled with a sufficient amount of the cryopreservation solution to cover the placental membrane. The amount of the cryopreservation solution necessary can depend on a number of factors including, for example, the type of container and mounting used as well as the size of the membranes to be preserved. The lower the amount of cryopreservation solution necessary to top (or cover) the composition/device, the faster the composition is able to thaw. Thus, it is desirable to use the least amount of cryopreservation solution that allows for top coverage of the membrane without compromising viability of the cells during the freeze thaw. Further, the smaller the membrane and the smaller the container used, the less cryopreservation solution can be used.

In some embodiments, a bag is used containing cryopreservation solution in an amount from about 7 mL to about 50 ml, alternatively from about 10 mL to about 50 ml, alternatively from about 15 mL to about 50 ml, alternatively from about 15 mL to about 25 ml. In one preferred embodiment, about 15 mL of cryopreservation solution is added to the container or bag. The amount of cryopreservation solution can be sufficient to fully submerge the membrane. The amount will depend on the size of the bag used and the size of the membrane being cryopreserved. If a small bag is being used with a small (e.g. smaller than 2 cm×2 cm membrane), about 3 mL to about 10 ml, alternatively 3 mL to about 7 mL of cryopreservation solution may be used.

In some embodiments a container is used containing from about 7 mL to about 50 ml, alternatively from about 5 mL to about 20 ml, alternatively from about 7 mL to about 20 ml, alternatively from about 7 mL to about 15 ml. The amount of cryopreservation solution can be sufficient to fully submerge the membrane within the container. The amount will depend on the size of the container used and the size of the membrane being cryopreserved.

In some embodiments, the amount of cryopreservation solution is sufficient to protect cells during the freezing and subsequent thawing procedures. In some embodiments, at least 70% cell viability is maintained after a freeze-thaw. In some aspects, at least 75% cell viability is maintained, alternatively about 80% cell viability is maintained, alternatively 85% cell viability is maintained, alternatively about 90% cell viability is maintained, alternatively about 95% cell viability is maintained, alternatively about 100% cell viability is maintained. In some embodiments, at viability of the membrane is at least 70%, at least 71%, at least 72%, at least 73%, at least 74%, at least 75%, at least 75%, at least 78%, at least 80%, at least 82%, at least 85%, at least 88%, at least 89%, at least 90%, at least 92%, and percentages in between.

In some embodiments, the amount of cryopreservation solution is sufficient to protect the structural, architectural, and/or 3-D structure of the membrane. In these instances, the cryopreservation solution contains a cell-permeating cryopreservative in an amount of 0.01% to about 100%, alternatively from about 2% to about 100%. In some embodiments, the cryopreservation solution contains polysaccharides or monosaccharides.

In one embodiment, the placental product is placed on nitrocellulose paper.

In one embodiment, the placenta, amniotic, and/or chorionic membrane may be cut into a plurality of sections. Optionally, the membranes may be any suitable size and are customizable depending on the type of membrane and the particular end application or usage of that membrane. Suitable sizes of membrane include, but are not limited to, about 1.5 cm×about 1.5 cm, about 2 cm×about 2 cm, about 3 cm×about 3 cm, about 4 cm×about 4 cm, about 5 cm×about 5 cm, about 6 cm×about 6 cm, about 7 cm×about 7 cm, about 8 cm×about 8 cm, about 7.5 cm×about 15 cm, about 1.5 cm×about 2 cm, about 1.5 cm×about 3 cm, about 2 cm×about 3 cm, about 3 cm×about 4 cm, about 2 cm×about 5 cm, about 3 cm×about 5 cm, about 4 cm×about 5 cm, about 5 cm×about 7 cm, about 5 cm×about 10 cm, about 5 cm×about 15 cm, about 7.5 cm×about 15 cm and include any variations or sizes and ranges there between, in increment of 0.1 cm to 1 cm.

The cryopreserved membranes have a surprisingly long shelf-life or stability and retain viable cells when frozen for extended periods of time. The cryopreserved products may be stored at about −20° C. to about −196° C. (e.g., about −45 to about −80° C.) for two years or more with retention of high cell viability (at least 40%, 50%, 60%, or 70% or more retention of viable cells) once thawed. In some aspects, the cryopreserved membranes can be stored at about −20° C. to about −196° C. (e.g., about −45 to about −80° C.) for at least about 3 months, at least about 6 months, at least about 9 months, at least about 12 months, at least about 15 months, at least about 24 months, at least about 25 months, at least about 36 months, or more before thawing with a high retention of viable cells (e.g., at least 40% viable cells, alternatively at least 50% viable cells, alternatively at least about 70% viable cells, alternatively at least about 80%, about 85%, 90%, 95% or 100% viable cells.

In another aspect, the disclosure provides a kit for treating a wound or a tissue defect comprising:

A) a membrane according to any of the aspects and embodiments described herein, in a pharmaceutically acceptable container; and

B) instructions for administering the membrane for treating the wound or the tissue defect.

In embodiments, the kit may comprise an additive that is selected from one or more antibiotics, emollients, keratolytic agents, humectants, antioxidants, preservatives, therapeutics, bandages, tools, cutting device, buffer, thawing medium, handling media, forceps, container and combinations thereof.

### Manufacture

### Overview

A placental product of the present technology can be manufactured from a placenta in any suitable manner that provides the technical features taught herein. According to the present technology, a placenta product comprises at least an immunocompatible amniotic membrane.

In one embodiment, a placental product is manufactured by a method comprising:

a. obtaining a placenta,
b. selectively depleting the placental membrane of immunogenicity; and
c. cryopreserving the placental membrane.

Optionally, the method comprises a step of removing maternal blood cells from the placental membrane, for example, by lysing red blood cells, by removing blood clots, or a combination thereof.

Optionally, the method comprises a step of treating the placental membranes with one or more antibiotics.

Optionally, the method comprises a step of selectively depleting CD14+ macrophages, optionally as demonstrated by a substantial decrease in LPS stimulation of TNFα release.

Optionally, the step of cryopreserving the placental membranes comprises freezing the membrane in a cryopreservation solution which comprises one or more cell-permeating cryopreservatives, one or more non cell-permeating cryopreservatives, or a combination thereof.

Optionally, the step of cryopreserving the placental membranes comprises placing at about 2° C. to about 8° C. for a period of time and then freezing at −80° C., thereby selectively depleting CD14+ macrophages optionally as demonstrated by a substantial decrease in LPS stimulation of TNFα release.

Optionally, the method comprises retaining a layer of epithelial cells of the amniotic membrane.

Optionally, the method comprises a step of removing the chorionic membrane or portion thereof. Optionally, the method comprises removing trophoblasts from the chorionic membrane while retaining the stromal layer of the chorionic membrane.

An exemplary placental membrane of the present technology can be manufactured or provided with a bandage or wound dressing.

### Immunocompatability and Selective Depletion

In one embodiment, the technology the placental product or membrane is immunocompatible. Immunocompatability can be accomplished by any selective depletion step that removes immunogenic cells or factors or immunogenicity from the placenta (or amniotic membrane thereof).

In one embodiment, the placental product is made immunocompatible by selectively depleting it of functional immunogenic cells. A placenta can be made immunocompatible by selectively removing immunogenic cells from the placental product (or amniotic membrane thereof) while retaining the therapeutic cells. For example, immunogenic cells can be removed by killing the immunogenic cells or by purification of the placenta there from.

In one embodiment, the placental product is made immunocompatible by selectively depleting trophoblasts, for example, by removal of the trophoblast layer.

In one embodiment, the placental product is made immunocompatible by selective depletion of functional CD14+ macrophages, optionally as demonstrated by a substantial decrease in LPS stimulation of TNFα release or by MLR assay.

In one embodiment, the placental product is made immunocompatible by selective depletion of maternal blood cells.

In one embodiment, the placental product is made immunocompatible by selective depletion of functional CD14+ macrophages, trophoblasts, and maternal blood cells.

In one embodiment, the placental product is made immunocompatible by selective depletion of trophoblasts and/or CD14+ macrophages, optionally as demonstrated by a substantial decrease in LPS stimulation of TNFα release or by MLR assay.

### Trophoblast Removal

In one embodiment, immunocompatability (or selective depletion) is accomplished by removal or depletion of trophoblasts from the placental product. Removal of trophoblasts from the chorionic membrane is conducted while retaining the stromal layer of the chorionic membrane. Surprisingly, such a placental product has one or more of the following superior features:

a. is substantially non-immunogenic;
b. provides remarkable healing; and
c. provides enhanced therapeutic efficacy.

In one embodiment, trophoblasts are removed while retaining the stromal layer of the chorionic membrane.

Trophoblasts can be removed in any suitable manner which substantially diminishes the trophoblast content of the placental product. In one embodiment, the trophoblasts are selectively removed. Optionally, the trophoblasts are selectively removed or otherwise removed without eliminating a substantial portion of one or more therapeutic components from the chorionic membrane (e.g. MSCs, therapeutic factors, extracellular matrix etc). Optionally, a majority (e.g. substantially all) of the trophoblasts are removed.

One method of removing trophoblasts comprises treating the placenta (e.g. chorion or amnio-chorion) with a digestive enzyme such as dispase (e.g. dispase II) and separating the trophoblasts from the placenta. Optionally, the step of separating comprises mechanical separation such as peeling or scraping. Optionally, scraping comprises scraping with a soft instrument such as a finger.

One method of removing trophoblasts comprises treating the chorionic membrane with dispase for about 30 to about 45 minutes separating the trophoblasts from the placenta. Optionally, the dispase is provided in a solution of about less than about 1% (e.g. about 0.5%). Optionally, the step of separating comprises mechanical separation such as peeling or scraping. Optionally, scraping comprises scraping with a soft instrument such as a finger.

Useful methods of removing trophoblasts from a placenta (e.g. chorion) are described by Portmann-Lanz et al. (“Placental mesenchymal stem cells as potential autologous graft for pre- and perinatal neuroregeneration”; American Journal of Obstetrics and Gynecology (2006) 194, 664-73), (“Isolation and characterization of mesenchymal cells from human fetal membranes”; Journal Of Tissue Engineering And Regenerative Medicine 2007; 1: 296-305.), and (Concise Review: Isolation and Characterization of Cells from Human Term Placenta: Outcome of the First International Workshop on Placenta Derived Stem Cells”).

In one embodiment, trophoblasts are removed before cryopreservation.

### Macrophage Removal

In one embodiment, functional macrophages are depleted or removed from the placental product. Surprisingly, such a placental product has one or more of the following superior features:

a. is substantially non-immunogenic;
b. provides remarkable healing; and
c. provides enhanced therapeutic efficacy.

Functional macrophages can be removed in any suitable manner which substantially diminishes the macrophage content of the placental product. Optionally, the macrophages are selectively removed or otherwise removed without eliminating a substantial portion of one or more therapeutic components from the placenta (e.g. MSCs, therapeutic factors, etc). Optionally, a majority (e.g. substantially all) of the macrophages are removed.

One method of removing immune cells such as macrophages comprises killing the immune cells by rapid freezing rates such as 60-100° C./min. Another method of removing immune cells comprises killing the immune cells by holding the cells at about 2° C. to about 8° C. for a period of time, and then freezing the immune cells (e.g., at about −20° C.) at a rate of about 1° C./min.

Although immune cells can be eliminated by rapid freezing rates, such a method can also be detrimental to therapeutic cells such as stromal cells (e.g. MSCs). The present inventors have discovered a method of selectively killing CD14+ macrophages by placing the placental membrane at about 2° C. to about 8° C. for a period of time (e.g. for at least about 10 min such as for about 30-60 mins) at a temperature above freezing (e.g. incubating at 2-8° C.) and then freezing the placenta (e.g. incubating at −80° C.±5° C.). Optionally, the step of freezing comprises freezing at a rate of less than 10°/min (e.g. less than about 5°/min such as at about 1°/min).

In one embodiment, the step of placing the placental membrane at about 2° C. to about 8° C. comprises soaking the placental membrane in a cryopreservation solution (e.g. containing DMSO) for a period of time sufficient to allow the cryopreservation solution to penetrate (e.g. equilibrate with) the placental tissues. Optionally, the step of freezing comprises reducing the temperature at a rate of about 1°/min. Optionally, the step of freezing comprises freezing at a rate of less than 10°/min (e.g. less than about 5°/min such as at about 1°/min).

In one embodiment, the step of placing the placental membrane at about 2° C. to about 8° C. comprises soaking the placental membrane in a cryopreservation solution (e.g. containing DMSO) at a temperature of about −10-15° C. (e.g. at 2-8° C.) for at least about any of: 10 min, 20 min, 30 min, 40 min, or 50 min. In another embodiment the step of placing the placental membrane at about 2° C. to about 8° C. comprises soaking the placental membrane in a cryopreservation solution (e.g. containing DMSO) at a temperature of about −10-15° C. (e.g. at 2-8° C.) for about any of: 10-120, 20-90 min, or 30-60 min. Optionally, the step of freezing comprises freezing at a rate of less than 10°/min (e.g. less than about 5°/min such as at about 1°/min).

### Removal of Maternal Blood Cells

In one embodiment, maternal blood cells are depleted or removed from the placental product. Surprisingly, such a placental product has one or more of the following superior features:

a. is substantially non-immunogenic;

b. provides remarkable healing; and

c. provides enhanced therapeutic efficacy.

Maternal blood cells can be removed in any suitable manner which substantially diminishes such cell content of the placental product. Optionally, the maternal blood cells are selectively removed or otherwise removed without eliminating a substantial portion of one or more therapeutic components from the placenta (e.g. therapeutic cells (e.g., MSCs), therapeutic factors, including angiogenic factors, antioxidant agents, anti-inflammatory agents, etc).

In one embodiment, removal of maternal blood cells comprises rinsing the amniotic membrane (e.g. with buffer such as PBS) to remove gross blood clots and any excess blood cells.

In one embodiment, removal of maternal blood cells comprises treating the amniotic membrane with an anticoagulant (e.g. citrate dextrose solution).

In one embodiment, removal of maternal blood cells comprises rinsing the amniotic membrane (e.g. with buffer such as PBS) to remove gross blood clots and any excess blood cells, and treating the amniotic membrane with an anticoagulant (e.g. citrate dextrose solution).

In one embodiment, the chorionic membrane is retained and removal of maternal blood cells comprises separating the chorion from the placenta by cutting around the placental skirt on the side opposite of the umbilical cord. The chorion on the umbilical side of the placenta is not removed due to the vascularization on this side.

In one embodiment, the chorionic membrane is retained and removal of maternal blood cells comprises separating the chorion from the placenta by cutting around the placental skirt on the side opposite of the umbilical cord and rinsing the amniotic membrane and chorionic membrane (e.g. with buffer such as PBS) to remove gross blood clots and any excess blood cells.

In one embodiment, the chorionic membrane is retained and removal of maternal blood cells comprises separating the chorion from the placenta by cutting around the placental skirt on the side opposite of the umbilical cord and treating the amniotic membrane and chorionic membrane with an anticoagulant (e.g. citrate dextrose solution).

In one embodiment, the chorionic membrane is retained and removal of maternal blood cells comprises separating the chorion from the placenta by cutting around the placental skirt on the side opposite of the umbilical cord, rinsing the chorionic membrane amniotic membrane (e.g. with buffer such as PBS) to remove gross blood clots and any excess blood cells, and treating the amniotic membrane with an anticoagulant (e.g. citrate dextrose solution).

### Selective Depletion of Immunogenicity as Demonstrated by a Substantial Decrease in LPS Stimulation of TNFα Release.

In one embodiment, the placental product is selectively depleted of immunogenicity as demonstrated by a reduction in LPS stimulated TNF-α release. In one embodiment, the placental product is selectively depleted of macrophages.

In one embodiment, TNF-α is depleted by killing or removal of macrophages.

In one embodiment, TNF-α is functionally depleted by treatment with PGE2, which suppresses TNF-α secretion.

In some embodiments, TNF-α is inhibited at least about 80%, at least about 85%, at least about 90%, at least about 95%, or at least about 100%.

### Preservation

A placental product of the present technology may be used fresh or may be preserved for a period of time. Surprisingly, cryopreservation in the instant technology results in immunocompatible placental products.

In one embodiment, a placental product is cryopreserved. A placental product may be cryopreserved by incubation at freezing temperatures (from about −20° C. to about −196° C., e.g. a −80° C.±5° C.) in a cryopreservative solution.

Cryopreservation can comprise, for example, incubating the placental product at about 2° C. to about 8° C. for 30-60 min, and then incubating at −80° C. until use. The placental product may then be thawed for use. Optionally, the placental product is cryopreserved in a manner such that cell viability is retained surprisingly well after a freeze-thaw cycle.

In one embodiment, cryopreservation comprises storage in a cryopreservation solution comprising one or more cell-permeating cryopreservatives, one or more non cell-permeating cryopreservatives, or a combination thereof. Optionally, the cryopreservation solution comprises one or more cell-permeating cryopreservatives selected from DMSO, a glycerol, a glycol, a propylene glycol, an ethylene glycol, or a combination thereof. Optionally, the cryopreservation solution comprises one or more non cell-permeating cryopreservatives selected from polyvinylpyrrolidone, a hydroxyethyl starch, a polysaccharide, a monosaccharides, a sugar alcohol, an alginate, a trehalose, a raffinose, a dextran, or a combination thereof. Other examples of useful cryopreservatives are described in “Cryopreservation” (BioFiles Volume 5 Number 4—Sigma-Aldrich® datasheet).

In one embodiment, the cryopreservation solution comprises a cell-permeating cryopreservative, wherein the majority of the cell-permeating cryopreservative is DMSO. Optionally, the cryopreservation solution does not comprise a substantial amount of glycerol.

In one embodiment, the cryopreservation solution comprises DMSO. Optionally, the cryopreservation solution does not comprise glycerol in a majority amount. Optionally, the cryopreservation solution does not comprise a substantial amount of glycerol.

In one embodiment, the cryopreservation solution comprises additional components such as albumin (e.g. HSA or BSA), an electrolyte solution (e.g. Plasma-Lyte), saline solution, or any combination thereof.

In some embodiments, the composition comprises cryopreservation solution. The cryopreservation solution is added to the container containing the membrane-mounted device or composition. Preferably, a sufficient amount of cryopreservation solution is added to the container to protect the membrane during the subsequent freezing steps. Suitable cryopreservation solutions are known in the art. In one embodiment, the cryopreservation comprises storage in a cryopreservation solution comprising one or more cell-permeating cryopreservatives, one or more non-cell permeating cryopreservatives, or a combination thereof. Suitable cryopreservatives include, but are not limited to, DMSO, a glycerol, a glycol, a propylene glycol, an ethylene glycol, propanediol, polyethylene glycol (PEG), 1,2-propanediol (PROH) or a combination thereof. In some embodiments, the cryopreservation solution may contain one or more non-cell permeating cryopreservative selected from polyvinyl pyrrolidione, a hydroxyethyl starch, a polysaccharide, a monosaccharide, an alginate, trehalose, raffinose, dextran, human serum albumin, ficoll, lipoproteins, polyvinyl pyrrolidone, hydroxyethyl starch, autologous plasma or a combination thereof. Other examples of useful cryopreservatives are described in Cryopreservation (BioFiles, Volume 5, Number 4 Sigma-Aldrich® Datasheet).

For example, a suitable cryopreservation solution comprises a cell-permeating cryopreservative, in an amount of at least about 0.001% to 100%, suitably in an amount from about 2% to about 20%, preferably about 5% to about 10% by volume. In some instances, the cryopreservation solution comprises at least about 2% cell-permeating cryopreservative. Further, the cryopreservation solution may comprise serum albumin or other suitable proteins. In some embodiments, the cryopreservation solution comprises from about 1% to about 20% serum albumin or other suitable proteins, alternatively from about 1% to about 10%. In one embodiment, the cryopreservation solution comprises 1% to about 15% albumin by volume and about 5% to about 20% cryopreservative by volume (e.g. about 10%). Optionally, the cryopreservative comprises DMSO (e.g. in a majority amount). Serum albumin or other suitable proteins are present to help stabilize the membrane during the freeze-thaw process and to reduce the damage to cells in order to maintain viability. Serum albumin may be human serum albumin or bovine serum albumin. The cryopreservation solution may further comprise a physiological buffer or saline, for example, phosphate buffer saline.

In one embodiment, cryopreservation comprises placing the placental membrane on nitrocellulose paper.

In one embodiment, the placental membrane is cut into a plurality of sections before cryopreservation. Optionally, the sections are placed on nitrocellulose paper before placing the placental membrane sections at about 2° C. to about 8° C.

### Methods of Use—

As discussed above, the placental products, and particularly the cryopreserved membranes described herein (e.g., amniotic, chorionic, and/or chorioamniotic membranes) provide an amount of viable cells, therapeutic factors, and extracellular matrix that are effective to promote a number of beneficial therapeutic activities and effects. In the methods disclosed herein the membranes may be applied and provide an environment that can promote cells to produce any number of therapeutic factors, as well as provide amounts of therapeutic factors, viable cells, and extracellular matrix that provide the same or similar therapeutic benefit.

Thus, in one aspect, the disclosure provides a method of treating a wound on a subject comprising administering a membrane comprising a cryopreserved amniotic membrane as described herein, wherein upon administration the membrane provides the viable cells, extracellular matrix, and/or one or more therapeutic factors in an amount effective to promote one or more of:

(i) a reduction of the amount and/or activity of pro-inflammatory cytokines;

(ii) an increase in the amount and/or activity of anti-inflammatory cytokines;

(iii) a reduction of the amount and/or activity of reactive oxygen species;

(iv) an increase in the amount and/or activity of antioxidant agents;

(v) a reduction of the amount and/or activity of proteases;

(vi) an increase in cell proliferation;

(vii) an increase in angiogenesis; and/or

(viii) an increase in cell migration.

In another aspect, the disclosure provides a method for accelerating wound healing comprising administering a membrane according to any of the aspects and embodiments described herein. In some embodiments, the administering is effective to promote wound closure by 12 weeks after an initial administering step. In some embodiments, the administering is effective to promote wound closure by 5-6 weeks after an initial administering step. In some embodiments, the administering is effective to promote reduction in wound size by 50% or more 28 days after an initial administering step. In embodiments, the administering is effective to improve wound closure rate by at least about 44% relative to standard wound treatment.

Standard wound treatment or standard of care for wound treatment may refer to and include any treatment that does not incorporate a membrane or product as described herein. Suitable standard wound treatments are known in the art and include, but are not limited to, application of dressings (e.g., gauze, bandages, barriers, or bioengineered membranes that contain no detectable or low numbers of viable cells), debridement, antibiotics, salves, ointment, and the like and any combinations thereof. Standard wound treatment may include debridement in conjunction with a wound dressing or bandage and an offloading device.

In another embodiment, the disclosure provides a method of treating a subject for a wound that is refractory to a prior wound healing treatment, the method comprising administering at or near the site of the wound a membrane according to any of the aspects and embodiments described herein. In some embodiments, the administering is effective to promote wound closure by 12 weeks after an initial administering step. In some embodiments, the administering is effective to promote wound closure by 5-6 weeks after an initial administering step. In some embodiments, the administering is effective to promote reduction in wound size by 50% or more 28 days after an initial administering step.

In another aspect, the disclosure provides a method for treating a chronic wound comprising administering to the site of the wound a membrane as disclosed herein wherein the administering provides the viable therapeutic cells, extracellular matrix, and one or more therapeutic factors in an amount effective to promote healing of the chronic wound.

In embodiments of the above aspects relating to wound treatment, accelerated wound treatment, and/or chronic wound treatment, the wound is selected from the group consisting of lacerations, scrapes, burns, incisions, punctures, wound caused by a projectile, an epidermal wound, skin wound, chronic wound, acute wound, external wound, internal wound, congenital wound, ulcer, pressure ulcer, diabetic ulcer, tunnel wound, wound caused during or as an adjunct to a surgical procedure, venous skin ulcer, and avascular necrosis.

In embodiments of the above aspects relating to wound healing methods, the membrane may either directly or may indirectly promote one or more of: (i) a reduction of the amount and/or activity of pro-inflammatory cytokines; (ii) an increase in the amount and/or activity of anti-inflammatory cytokines; (iii) a reduction of the amount and/or activity of reactive oxygen species; (iv) an increase in the amount and/or activity of antioxidant agents; (v) a reduction of the amount and/or activity of proteases; (vi) an increase in cell proliferation; (vii) an increase in angiogenesis; and/or (viii) an increase in cell migration.

In another aspect, the disclosure provides a method of treating an inflammatory ocular condition in a subject comprising administering to the subject a membrane as disclosed herein, wherein the administration provides the viable therapeutic cells, extracellular matrix, and one or more therapeutic factors in an amount effective to treat the inflammatory ocular condition. In embodiments of this aspect the method can comprise administration of the membrane using any technique that may be directed to promote epithelialization, reduce pain, and/or to generally reduce inflammation of eye tissue. For example the membrane can be administered via a surgical inlay or grafting technique, via an onlay or patching technique, or via a combination of inlay and onlay techniques. Generally, the method may be associated with eye surgery (e.g., photorefractive keratectomy (PRK)), eye trauma (e.g., lacerations, burns, or scrapes), or an eye disease that is characterized by inflammation or the treatment of which may result in an amount of inflammation in ocular tissue. Non-limiting examples of indications that include an “inflammatory ocular condition” encompassed by the method include general repair/reconstruction of the corneal or conjunctiva surface(s) such as, for example, persistent epithelial defects; corneal ulceration; corneal transplant; descemetocele; corneal perforations; defects following excision of epithelial or subepithelial lesions or tumors (conjunctival tumors, conjunctival intraeptithelial neoplasia, subepithelial lesions, band keratopathy, scars, conjunctival folds parallel to the edges of eyelids); acute chemical burns; acute keratitis; painful bullous keratopathy; partial or complete limbal stem cell deficiency (with stem cell grafting); acute Stevens-Johnson syndrome; symbelpharon; formix reconstruction; anophthalmia; bleb revisions; scleral thinning; and pterygium (see, e.g., Meller, D., et al., Dtsch. Arztebl. Int., (2011); 108(14):243-248, incorporated herein by reference).

In another aspect, the disclosure provides a method of promoting tissue repair and/or tissue regeneration in a subject comprising administering to the subject a membrane as disclosed herein wherein the administration provides the viable therapeutic cells, extracellular matrix, and one or more therapeutic factors in an amount effective to promote tissue repair and/or tissue regeneration. In some embodiments of this aspect, the method is used in combination with a surgical procedure selected from the group consisting of a tissue graft procedure, tendon surgery, ligament surgery, bone surgery, and spinal surgery. In some embodiments, the tissue is human tissue. In further embodiments the human tissue is cartilage, skin, ligament, tendon, or bone. In this aspect and the various embodiments, the membrane may directly or indirectly stimulates tissue regeneration.

In another aspect, the disclosure provides a method of modulating inflammatory response comprising administering to the wound a membrane as disclosed herein wherein the administration provides the viable cells, extracellular matrix, and/or one or more therapeutic factors in an amount effective to reduce the amount and/or activity of pro-inflammatory cytokines, and/or increase the amount and/or activity of anti-inflammatory cytokines. In embodiments, the pro-inflammatory cytokine is selected from TNF-α and IL-1α, or a combination thereof. In other embodiments the anti-inflammatory cytokine may be selected from IL-10 or PGE2 or a combination thereof.

In an aspect, the disclosure provides a method of modulating protease activity comprising administering to the wound a membrane as described herein wherein the administration provides the viable cells, extracellular matrix, and/or one or more therapeutic factors in an amount effective to reduce protease activity, and/or increase the amount and/or activity of protease inhibitors. In embodiments, the protease may be selected from the group consisting of a matrix metalloproteinase (MMP) and elastase, or any combinations thereof. In some embodiments, the one or more protease inhibitors comprise a tissue inhibitor of matrix metalloproteinase (TIMP).

In a further aspect, the disclosure provides a method of reducing the amount and/or activity of reactive oxygen species (ROS) comprising administering to the wound a membrane as described herein, wherein the administration provides the viable cells, extracellular matrix, and/or one or more therapeutic factors in an amount effective to reduce the amount and/or activity of ROS and/or increase the amount and/or activity of antioxidant agents. In some embodiments the antioxidant capacity provided by the membrane may be equivalent to up to about a 250 mM solution of ascorbic acid. In some embodiments, the membrane may rescue cells from oxidant-induced apoptosis. In some embodiments, the membrane may reduce or prevent cells from undergoing apoptosis after being exposed to an oxidant and/or undergoing oxidative injury. In some aspect, the products are able to reduce the amount of cells that undergo oxidant-induced apoptosis by at least about 50%, alternatively at least about 60%, alternatively at least about 70%, alternatively at least about 80%, alternatively at least about 90%.

In another aspect, the disclosure provides a method of increasing angiogenesis comprising administering to wound a membrane as described herein, wherein the administration provides the viable cells, extracellular matrix, and/or one or more therapeutic factors in an amount effective to increase angiogenesis. In some embodiments the membrane provides an increase the amount and/or activity of a vascular endothelial growth factor (VEGF) or epidermal growth factor (EGF) or a combination thereof. In some embodiments, the membrane promotes vessel formation.

In some embodiments, the membrane is able to promote and/or enhance the formation of closed tubes or vessels in a tissue. In vitro, membranes promote the formation of closed tubes by HUVECs.

In another aspect, the disclosure provides a method of increasing cell migration comprising administering to the wound a membrane as described herein, wherein the administration provides the viable cells, extracellular matrix, and/or one or more therapeutic factors in an amount effective increase cell migration. In some embodiments, the membrane induces the cell migration of cells selected from the group consisting of endothelial cells, fibroblasts, and epithelial cells, and combinations thereof.

In yet another aspect, the disclosure provides a method of increasing cell proliferation comprising administering to wound a membrane as described herein, wherein the administration provides the viable cells, extracellular matrix, and/or one or more therapeutic factors in an amount effective increase cell proliferation.

In a further aspect, the disclosure provides a method of preventing or reducing scar or contracture formation in a subject comprising administering to the subject a membrane as described herein, wherein the administration provides the viable therapeutic cells, extracellular matrix, and one or more therapeutic factors in an amount effective to prevent or reduce scar or contracture formation. In embodiments of this method the membrane may increase the amount and/or activity of IFN-2a and/or TGF-β3 in an amount effective to prevent or reduce scar formation.

In any of the above aspects relating to methods of modulating inflammatory response, modulating protease activity, reducing the amount of reactive oxygen species/increasing the amount of antioxidant agents, increasing/promoting angiogenesis, increasing cell migration, increasing cell proliferation, or preventing or reducing scar or contracture formation the administration of the membrane may directly or indirectly stimulate or induce the method.

The placental products of the present technology may be used to treat any tissue injury. A method of treatment may be provided, for example, by administering to a subject in need thereof, a placental product of the present technology.

A typical administration method of the present technology is topical administration. Administering the present technology can optionally involve administration to an internal tissue where access is gained by a surgical procedure.

Placental products can be administered autologously, allogeneically or xenogeneically.

In one embodiment, a present placental product is administered to a subject to treat a wound. Optionally, the wound is a laceration, scrape, thermal or chemical burn, incision, puncture, or wound caused by a projectile. Optionally, the wound is an epidermal wound, skin wound, chronic wound, acute wound, external wound, internal wounds, congenital wound, ulcer, or pressure ulcer. Such wounds may be accidental or deliberate, e.g., wounds caused during or as an adjunct to a surgical procedure. Optionally, the wound is closed surgically prior to administration.

In one embodiment, a present placental product is administered to a subject to treat a burn. Optionally, the burn is a first-degree burn, second-degree burn (partial thickness burns), third degree burn (full thickness burns), infection of burn wound, infection of excised and unexcised burn wound, loss of epithelium from a previously grafted or healed burn, or burn wound impetigo.

In one embodiment, a present placental product is administered to a subject to treat an ulcer, for example, a diabetic ulcer (e.g. foot ulcer).

In one embodiment, a placental product is administered by placing the placental product directly over the skin of the subject, e.g., on the stratum corneum, on the site of the wound, so that the wound is covered, for example, using an adhesive tape. Additionally or alternatively, the placental product may be administered as an implant, e.g., as a subcutaneous implant.

In one embodiment, a placental product is administered to the epidermis to reduce rhytids or other features of aging skin. Such treatment is also usefully combined with so-called cosmetic surgery (e.g. rhinoplasty, rhytidectomy, etc.).

In one embodiment, a placental product is administered to the epidermis to accelerate healing associated with a dermal ablation procedure or a dermal abrasion procedure (e.g. including laser ablation, thermal ablation, electric ablation, deep dermal ablation, sub-dermal ablation, fractional ablation, and microdermal abrasion).

Other pathologies that may be treated with placental products of the present technology include traumatic wounds (e.g. civilian and military wounds), surgical scars and wounds, spinal fusions, spinal cord injury, avascular necrosis, reconstructive surgeries, ablations, and ischemia.

In one embodiment, a placental product of the present technology is used in a tissue graft procedure. Optionally, the placental product is applied to a portion of the graft which is then attached to a biological substrate (e.g. to promote healing and/or attachment to the substrate). By way of non-limiting example, tissues such as skin, cartilage, ligament, tendon, periosteum, perichondrium, synovium, fascia, mesenter and sinew can be used as tissue graft.

In one embodiment, a placental product is used in a tendon or ligament surgery to promote healing of a tendon or ligament. Optionally, the placental product is applied to portion of a tendon or ligament which is attached to a bone. The surgery can be any tendon or ligament surgery, including, e.g. knee surgery, shoulder, leg surgery, arm surgery, elbow surgery, finger surgery, hand surgery, wrist surgery, toe surgery, foot surgery, ankle surgery, and the like. For example, the placental product can be applied to a tendon or ligament in a grafting or reconstruction procedure to promote fixation of the tendon or ligament to a bone.

Through the insight of the inventors, it has surprisingly been discovered that placental products of the present technology provide superior treatment (e.g. healing, healing time and/or healing strength) for tendon and ligament surgeries. Tendon and ligament surgeries can involve the fixation of the tendon or ligament to bone. Without being bound by theory, the present inventors believe that osteogenic and/or chondrogenic potential of MSCs in the present placental products promotes healing process and healing strength of tendons or ligaments. The present inventors believe that the present placental products provide an alternative or adjunctive treatment to periosteum-based therapies. For example, useful periosteum based treatments are described in Chen et al. (“Enveloping the tendon graft with periosteum to enhance tendon-bone healing in a bone tunnel: A biomechanical and histologic study in rabbits”; Arthroscopy. 2003 March; 19(3):290-6), Chen et al. (“Enveloping of periosteum on the hamstring tendon graft in anterior cruciate ligament reconstruction”; Arthroscopy. 2002 May-June; 18(5):27E), Chang et al. (“Rotator cuff repair with periosteum for enhancing tendon-bone healing: a biomechanical and histological study in rabbits”; Knee Surgery, Sports Traumatology, Arthroscopy Volume 17, Number 12, 1447-1453), each of which are incorporated by reference.

As non-limiting example of a method of tendon or ligament surgery, a tendon is sutured to and/or wrapped or enveloped in a placental membrane and the tendon is attached to a bone. Optionally, the tendon is placed into a bone tunnel before attached to the bone.

In one embodiment, the tendon or ligament surgery is a graft procedure, wherein the placental product is applied to the graft. Optionally, the graft is an allograft, xenograft, or an autologous graft.

In one embodiment, the tendon or ligament surgery is repair of a torn ligament or tendon, wherein the placental product is applied to the torn ligament or tendon.

Non-limiting examples of tendons to which a placental product can be applied include a digitorum extensor tendon, a hamstring tendon, a bicep tendon, an Achilles Tendon, an extensor tendon, and a rotator cuff tendon.

In one embodiment, a placental product of the present technology is used to reduce fibrosis by applying the placental product to a wound site.

In one embodiment, a placental product of the present technology is used as an anti-adhesion wound barrier, wherein the placental product is applied to a wound site, for example, to reduce fibrosis (e.g. postoperative fibrosis).

Non-limiting examples of wound sites to which the placental product can be applied include those that are surgically induced or associated with surgery involving the spine, laminectomy, knee, shoulder, or child birth, trauma related wounds or injuries, cardiovascular procedures, requiring angiogenesis stimulation, brain/neurological procedures, burn and wound care, and ophthalmic procedures. For example, optionally, the wound site is associated with surgery of the spine and the stromal side of the placental product is applied to the dura (e.g. the stromal side facing the dura). Direction for such procedures, including the selection of wound sites and/or methodologies, can be found, for example, in WO 2009/132186 and US 2010/0098743, which are hereby incorporated by reference.

A placental product of the present technology can optionally be used to reduce adhesion or fibrosis of a wound. Postoperative fibrosis is a natural consequence of all surgical wound healing. By example, postoperative peridural adhesion results in tethering, traction, and compression of the thecal sac and nerve roots, which cause a recurrence of hyperesthesia that typically manifests a few months after laminectomy surgery. Repeated surgery for removal of scar tissue is associated with poor outcome and increased risk of injury because of the difficulty of identifying neural structures that are surrounded by scar tissue. Therefore, experimental and clinical studies have primarily focused on preventing the adhesion of scar tissue to the dura matter and nerve roots. Spinal adhesions have been implicated as a major contributing factor in failure of spine surgery. Fibrotic scar tissue can cause compression and tethering of nerve roots, which can be associated with recurrent pain and physical impairment.

Without being bound by theory, the present inventors believe that placental products taught herein are useful to reduce adhesion or fibrosis of a wound, at least in part, because the placental products can function in-situ to provide an environment that includes reduced numbers of immune cells as well as an increased number of cellular factors (e.g., TGF-β3, HGF, VGF, EGF, HE, hyaluronic acid, etc.). One advantage of the wound dressings and processes of the present technology is that an anti-adhesion barrier is provided which can be used to prevent adhesions following surgery, and in particular following back surgery.

In the preceding paragraphs, use of the singular may include the plural except where specifically indicated. As used herein, the words “a,” “an,” and “the” mean “one or more,” unless otherwise specified. In addition, where aspects of the present technology are described with reference to lists of alternatives, the technology includes any individual member or subgroup of the list of alternatives and any combinations of one or more thereof.

The disclosures of all patents and publications, including published patent applications, are hereby incorporated by reference in their entireties to the same extent as if each patent and publication were specifically and individually incorporated by reference.

It is to be understood that the scope of the present technology is not to be limited to the specific embodiments described above. The present technology may be practiced other than as particularly described and still be within the scope of the accompanying claims.

Likewise, the following examples are presented in order to more fully illustrate the present technology. They should in no way be construed, however, as limiting the broad scope of the technology disclosed herein.

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

Other features and embodiments of the present technology will become apparent from the following examples which are given for illustration of the present technology rather than for limiting its intended scope.

### Manufacturing Process

### Example 1

### Exemplary Manufacturing Process of an Amniotic Product

In one embodiment, and as discussed herein, the disclosure relates to a method of manufacturing a placental product (or alternatively, a “membrane” in the examples that follow) comprising an amniotic membrane from placenta post-partum. One such method includes:
a. Remove umbilical cord close to placental surface,
b. Blunt dissect of the amnion to placental skirt,
c. Flip placenta over and completely remove amnion,
d. Rinse amnion in PBS to remove red blood cells,
e. Rinse amnion once with 11% ACD-A solution to assist in red blood cell removal,
f. Rinse amnion with PBS to remove ACD-A solution,
g. Use PBS to remove any remaining blood from the amnion,
h. Gently remove any other components that are not part of the epithelial and stromal layers of the amnion,
i. Place the amnion in PBS and set aside,
j. Place the amnion into a bottle containing antibiotic solution and incubate at 37° C.±2° C. for 24-28 hrs,
k. Remove bottle from the incubator and rinse membrane with PBS to remove antibiotic solution,
l. Mount amnion (epithelial side up) on reinforced nitrocellulose paper and cut to size,
m. Place into an FP-90 cryobag and heat seal,
n. Add 50 mL cryopreservation solution to the bag through a syringe and remove any air trapped within the bag with the syringe,
o. Tube seal the solution line on the FP-90 bag,
p. Place filled bag into secondary bag and heat seal,
q. Place unit into packaging carton,
r. Refrigerate at 2-8° C. for 30-60 minutes, Freeze at −80° C.±5° C. inside a Styrofoam container.

### Example 2

### Exemplary Manufacturing Process of a Placental Product Containing a Chorionic Membrane

One method of manufacturing a placental product comprising a chorionic membrane and optionally an amniotic membrane from placenta post-partum includes:
a. Remove umbilical cord close to placental surface,
b. Blunt dissect of the amnion to placental skirt,
c. Flip placenta over and completely remove amnion,
d. Remove chorion by cutting around placental skirt,
e. Rinse both membranes in PBS to remove red blood cells,
f. Rinse both membranes once with 11% ACD-A solution to assist in red blood cell removal,
g. Rinse both membranes with PBS to remove ACD-A solution,
h. Treat chorion in 200 ml 0.5% dispase solution at 37° C.±2° C. for 30-45 minutes,
i. When dispase treatment is complete, rinse chorion with PBS to remove dispase solution,
j. Gently remove trophoblast layer from the chorion,
k. Place chorion into a bottle containing antibiotic solution and incubate at 37° C.±2° C. for 24-28 hrs,
;. Remove bottles from the incubator and rinse chorion membrane with PBS to remove antibiotic solution,
m. Mount chorion on reinforced nitrocellulose paper and cut to size,
n. Place each piece into an FP-90 cryobag and heat seal,
o. Add 50 mL cryopreservation solution to the bag through a syringe and remove any air trapped within the bag with the syringe,
p. Tube seal the solution line on the FP-90 bag,
q. Place filled bag into secondary bag and heat seal,
r. Place unit into packaging carton,
s. Refrigerate at 2-8° C. for 30-60 minutes,
t. Freeze at −80° C.±5° C. inside a Styrofoam container.

### Example 3

### Exemplary Manufacturing Process of a Chorioamniotic Membrane Product

The disclosure provides a method of manufacturing a placental product comprising an amniotic membrane and a chorionic membrane from placenta post-partum that includes:
a. Remove umbilical cord close to placental surface,
b. Blunt dissect of the amnion to placental skirt,
c. Flip placenta over,
d. Remove chorion and attached amnion (chorioamniotic membrane) by cutting around placental skirt,
e. Rinse both membranes in PBS to remove red blood cells,
f. Rinse both membranes once with 11% ACD-A solution to assist in red blood cell removal,
g. Rinse both membranes with PBS to remove ACD-A solution,
h. Place the membranes into a bottle containing antibiotic solution and incubate at 37° C.±2° C. for 24-28 hrs,
i. Remove bottle from the incubator and rinse with PBS to remove antibiotic solution,
j. Gently remove the trophoblast layer from the chorion,
k. Mount chorioamniotic membrane on reinforced nitrocellulose paper and cut to size,
l. Place each piece into an FP-90 cryobag and heat seal,
m. Add 50 mL cryopreservation solution to the bag through a syringe and remove any air trapped within the bag with the syringe,
n. Tube seal the solution line on the FP-90 bag,
o. Place filled bag into secondary bag and heat seal,
p. Place unit into packaging carton,
q. Refrigerate at 2-8° C. for 30-60 minutes,
r. Freeze at −80° C.±5° C. inside a Styrofoam container.

### Example 4

### Exemplary Placental Product Manufacturing Process

Further details regarding one method for manufacturing a placental product comprising an amniotic membrane according to the presently disclosed manufacturing procedure are provided below.

The placenta was processed inside a biological safety cabinet. The umbilical cord was first removed, and the amniotic membrane was peeled from the underlying chorionic membrane using blunt dissection. The membrane was rinsed with phosphate buffered saline (PBS) (Gibco Invitrogen, Grand Island, N.Y.) to remove gross blood clots and any excess blood cells. The membrane was then washed with 11% anticoagulant citrate dextrose solution (USP) formula A (ACD-A) (Baxter Healthcare Corp., Deerfield, Ill.) in saline (Baxter Healthcare Corp., Deerfield, Ill.) to remove remaining blood cells.

The stromal side of the amnion was cleaned by gently scraping away any other components that are not part of the epithelial and stromal layers.

The amnion was then each disinfected in vented flasks with 250 mL of antibiotic solution consisting of gentamicin sulfate (50 μg/mL) (Abraxis Pharmaceutical Products, Schaumburg, Ill.), vancomycin HCl (50 μg/mL) (Hospira Inc., Lake Forest, Ill.), and amphotericin B (2.5 μg/mL) (Sigma Aldrich, St. Louis, Mo.) in Dulbecco's Modified Eagle Medium (DMEM) at 37° C.±2° C. for 24-28 hours. After the incubation period, the membrane was washed with PBS to remove any residual antibiotic solution.

The membrane was mounted on Optitran BA-S 85 reinforced nitrocellulose paper (Whatman, Dassel, Germany) and cut to the appropriate size prior to packaging into an FP-90 cryobag (Charter Medical Ltd., Winston-Salem, N.C.). The stromal side of the amnion was mounted towards the nitrocellulose paper. Once the membrane unit was placed into the FP-90 cryobag and the cryobag was heat sealed, 50 mL of a cryopreservation solution containing 10% dimethyl sulfoxide (DMSO) (Bioniche Teo. Inverin Co., Galway, Ireland) and 5% human serum albumin (HSA) (Baxter, West Lake Village, Calif.) in PlasmaLyte-A (Baxter Healthcare Corp., Deerfield, Ill.) were added through the center tubing line. Any excess air was removed, and the tubing line was subsequently sealed.

The FP-90 cryobag was placed into a mangar bag (10 in.×6 in.) (Mangar Industries, New Britain, Pa.), which was then heat sealed. The mangar bag was placed into a packaging carton (10.5 in.×6.5 in.×0.6 in.) (Diamond Packaging, Rochester, N.Y.). All cartons were refrigerated at 2-8° C. for 30-60 minutes prior to freezing at −80° C.±5° C. inside a Styrofoam container.

### Example 4.1

### Thawing Time for Membrane Mounted on Nitrocellulose Paper

The cryopreserved membranes described herein can exhibit thawing properties that are more rapid than other cryopreserved products. A rapid thaw profile allows for the membranes described herein to be used more efficiently and effectively for on-demand uses and application. One thawing protocol is discussed below.
Take 2 samples from 2 lots of placental tissue packaged on nitrocellulose paper out from the deep freezer (−80° C.).
Put cryobag containing placental tissue in the thawing basin filled with room temperature water.
Record thawing time, determined when no observable ice crystals remain.

TABLE 1
Thawing time for placental membrane products cryopreserved
with 50 mL cryopreservation solution.

| Donor | Thawing Time (min) |
|---|---|
| A | 24 |
| B | 28 |
| Average | 26 |

For thawing to be complete, all ice crystals have to disappear. Otherwise, the weight of the ice may tear the membrane or cause membrane to fall off of the nitrocellulose paper leading to self-folding when thawing is complete. Results are depicted in the Table 1 above, and demonstrate that average thawing time was 26 minutes, thus facilitating “on-demand” uses and applications for the cryopreserved membranes described herein.

### Example 5

### Exemplary Manufacturing Process of a Chorioamniotic Membrane Product

One method of manufacturing a placental product comprising an amniotic membrane product and a chorionic membrane product according to the presently disclosed manufacturing procedure is as follows:

The placenta was processed inside a biological safety cabinet. The umbilical cord was first removed, and the chorion with attached amniotic membrane was cut from the placental skirt. Both membranes were rinsed with phosphate buffered saline (PBS) (Gibco Invitrogen, Grand Island, N.Y.) to remove gross blood clots and any excess blood cells. The membranes were then washed with 11% anticoagulant citrate dextrose solution (USP) formula A (ACD-A) (Baxter Healthcare Corp., Deerfield, Ill.) in saline (Baxter Healthcare Corp., Deerfield, Ill.) to remove remaining blood cells.

The chorioamniotic membrane was then disinfected in vented flasks with 500 mL of antibiotic solution consisting of gentamicin sulfate (50 μg/mL) (Abraxis Pharmaceutical Products, Schaumburg, Ill.), vancomycin HCl (50 μg/mL) (Hospira Inc., Lake Forest, Ill.), and amphotericin B (2.5 μg/mL) (Sigma Aldrich, St. Louis, Mo.) in DMEM at 37° C.±2° C. for 24-28 hours. After the incubation period, the membranes were washed with PBS to remove any residual antibiotic solution. The trophoblast layer of the chorion was gently removed using blunt dissection.

The membranes were mounted on Optitran BA-S 85 reinforced nitrocellulose paper (Whatman, Dassel, Germany) and cut to the appropriate size prior to packaging into an FP-90 cryobag (Charter Medical Ltd., Winston-Salem, N.C. Once a membrane unit was placed into the FP-90 cryobag and the cryobag was heat sealed, 50 mL of a cryopreservation solution containing 10% dimethyl sulfoxide (DMSO) (Bioniche Teo. Inverin Co., Galway, Ireland) and 5% human serum albumin (HSA) (Baxter, West Lake Village, Calif.) in PlasmaLyte-A (Baxter Healthcare Corp., Deerfield, Ill.) were added through the center tubing line. Any excess air was removed, and the tubing line was subsequently sealed.

The FP-90 cryobag was placed into a mangar bag (10 in.×6 in.) (Mangar Industries, New Britain, Pa.), which was then heat sealed. The mangar bag was placed into a packaging carton (10.5 in.×6.5 in.×0.6 in.) (Diamond Packaging, Rochester, N.Y.). All cartons were refrigerated at 2-8° C. for 30-60 minutes prior to freezing at −80° C.±5° C. inside a Styrofoam container.

### Example 6

### Quantitative Evaluation of Cell Number and Cell Viability after Enzymatic Digestion of Placental Membranes

Amnion and chorion membranes and present placental membrane products (from above) were evaluated for cell number and cell viability throughout the process. These analyses were performed on fresh placental tissue (prior to the antibiotic treatment step), placental tissue post antibiotic treatment, and product units post thaw. Cells were isolated from the placental membranes using enzymatic digestion. For the cryopreserved product units, the FP-90 cryobags were first removed from the packaging cartons and mangar bags. Then the FP-90 cryobags were thawed for 2-3 minutes in a room temperature water bath. Early experiments involved the use of a 37° C.±2° C. water bath. After thaw, the placental membranes were removed from the FP-90 cryobag and placed into a reservoir containing saline (Baxter Healthcare Corp., Deerfield, Ill.) for a minimum of 1 minute and a maximum of 60 minutes. Each membrane was detached from the reinforced nitrocellulose paper prior to digestion.

Amniotic membranes were digested with 40 mL of 0.75% collagenase (Worthington Biochemical Corp., Lakewood, N.J.) solution at 37° C.±2° C. for 20-40 minutes on a rocker. After collagenase digestion, the samples were centrifuged at 2000 rpm for 10 minutes. The supernatant was removed, and 30 mL of 0.05% trypsin-EDTA (Lonza, Walkersville, Md.) were added and incubated at 37° C.±2° C. for an additional 5-15 minutes on a rocker. The trypsin was warmed to 37° C.±2° C. in a water bath prior to use. After trypsin digestion, the suspension was filtered through a 100 μm cell strainer nylon filter to remove any debris. Pass 15 ml DMEM through the strainer into the same conical tube. Centrifugation at 2000 rpm for 10 minutes was performed, and supernatant was removed. Cell pellets were reconstituted with a volume of DMEM that was proportional to the pellet size, and 20 μL of the resuspended cell suspension were mixed with 80 μL of trypan blue (Sigma Aldrich, St. Louis, Mo.) for counting. The cell count sample was placed into a hemocytometer and evaluated using a microscope.

Chorionic membranes were digested with 25 mL of 0.75% collagenase solution at 37° C.±2° C. for 20-40 minutes on a rocker. After collagenase digestion, the suspension was filtered through a 100 μm cell strainer nylon filter to remove any debris. Centrifugation at 2000 rpm for 10 minutes was performed, and supernatant was removed. Cell pellets were reconstituted with a volume of DMEM that was proportional to the pellet size, and 20 μL of the resuspended cell suspension were mixed with 80 μL of trypan blue for counting. The cell count sample was placed into a hemocytometer and evaluated using a microscope.

Placenta membranes were analyzed prior to any processing to determine the initial characteristics of the membranes

Table 2 contains average cells per cm² and cell viability values for the amniotic and chorionic membranes from 32 placenta lots.

On average, there were 91,381 viable cells per cm² for the amniotic membrane with a corresponding average cell viability of 84.5%. For the chorionic membrane, there were 51,614 viable cells per cm² with a corresponding cell viability of 86.0%.

TABLE 2
Viable cells per cm² and cell viability values
from fresh placental tissue from 32 donors.

| | Viable Cells per cm² - | % Cell Viability - |
|---|---|---|
| | Fresh tissue | Fresh tissue |
| Membrane | (Average ± SD) | (Average ± SD) |
| Amnion | 91,381 ± 49,597 | 84.5% ± 3.7% |
| Chorion | 51,614 ± 25,478 | 86.0% ± 4.7% |

These data illustrate cell numbers that are useful with certain embodiments of the present technology; e.g. placental product comprising an amniotic membrane containing about 10,000 to about 360,000 cells/cm². Since the amniotic membrane consists of epithelial cells and stromal cells, experiments were conducted to determine the ratio of epithelial cells to stromal cells. Amniotic membranes from 3 placenta lots were analyzed. First, a 5 cm×5 cm piece of amniotic membrane was digested with approximately 25 mL of 0.05% trypsin-EDTA (Lonza, Walkersville, Md.) at 37° C.±2° C. in a water bath for 30 minutes. After the incubation step, epithelial cells were removed by gently scraping the cells from the membrane. After rinsing with PBS (Gibco Invitrogen, Grand Island, N.Y.), the membrane was subsequently digested in the same manner as chorionic membrane (described above). In addition, another intact 5 cm×5 cm piece of amniotic membrane was digested using the standard procedure (described above) to determine the total number of cells. The percentage of stromal cells was then determined by dividing the cell count from the amniotic membrane with the epithelial cells removed with the cell count from the intact membrane.

Results indicate that 19% of the total cells were stromal cells. Therefore, approximately 17,362 stromal cells were present in amniotic membrane with approximately 74,019 epithelial cells. These data indicated that there are approximately 3 times more stromal cells in chorionic membranes as compared to amniotic membranes. This ratio is consistent with certain embodiments of the present technology that provide a placental product comprising a chorionic membrane and an amniotic membrane, wherein the chorionic membrane comprises about 2 to about 4 times more stromal cells relative to the amniotic membrane.

Viable cells per cm² and cell viability were assessed after the antibiotic treatment step. Process cell recovery was calculated by comparing the number of viable cells before and after the antibiotic process (as described previously in this presently described technology). Table 3 provides the results from these analyses.

TABLE 3
Viable cells per cm² and cell viability for post
antibiotic placental tissue from 28 donors.

| | Viable Cells per | % Cell Viability | % Process Cell |
|---|---|---|---|
| | cm² post antibiotic | post antibiotic | Recovery |
| | treatment | treatment | (Process/Fresh*100) |
| Membrane | (Average ± SD) | (Average ± SD) | (Average ± SD) |
| Amnion | 75,230 ± 46,890 | 84.4% ± 4.2% | 87.7% ± 49.4% |
| Chorion | 33,028 ± 18,595 | 85.6% ± 4.4% | 70.3% ± 31.1% |

### Example 7

### Development of a Placental Product Cryopreservation Procedure

Cryopreservation is a method that provides a source of tissues and living cells. A main objective of cryopreservation is to minimize damage to biological materials during low temperature freezing and storage. Although general cryopreservation rules are applicable to all cells, tissues, and organs, a specific cryopreservation solution and procedure must be developed for each type of biological material. The present application discloses a cryopreservation procedure for placental membrane products that can selectively deplete immunogenic cells from the placental membranes and preserve viability of other beneficial cells that are the primary source of factors for the promotion of healing.

During cryopreservation method development for placental membranes, the present inventors evaluated key parameters of cryopreservation including volume of cryopreservation solution, effect of tissue equilibration prior to freezing, and cooling rates for a freezing procedures.

Acceptance of tissue allografts in the absence of immunosuppression will depend on the number of satellite immune cells present in the tissue. Cryopreservation is an approach which can be utilized to reduce tissue immunogenicity. This approach is based on differential susceptibility of different cell types to freezing injury in the presence of DMSO; leukocytes are sensitive to fast cooling rates. The freezing rate of 1° C./min is considered optimal for cells and tissues including immune cells. Rapid freezing rates such as 60-100° C./min eliminate immune cells. However, this type of procedure is harmful to other tissue cells, which are desirable for preservation according to the present technology. The developed cryopreservation procedure utilized a cryopreservation solution containing 10% DMSO, which is a key component protecting cells from destruction when water forms crystals at low temperatures. The second step of cryopreservation was full equilibration of placental membrane in the cryopreservation solution, which was achieved by soaking membranes in the cryopreservation solution for 30-60 min at 4° C. This step allowed DMSO to penetrate the placental tissues. Some data indicates that tissue equilibration prior to freezing may affect survival of lymphocytes (e.g., Taylor & Bank, Cryobiology, 1988, 25:1); however, this data shows that a substantial number of macrophages survive even after tissue equilibration.

It was unexpectedly found that 30-60 min placental membrane equilibration in a DMSO-containing solution at about 2° C. to about 8° C. increases sensitivity of immune cells such as macrophages to freezing such that these types of cells cannot withstand freezing even at rates of about 1° C./min.

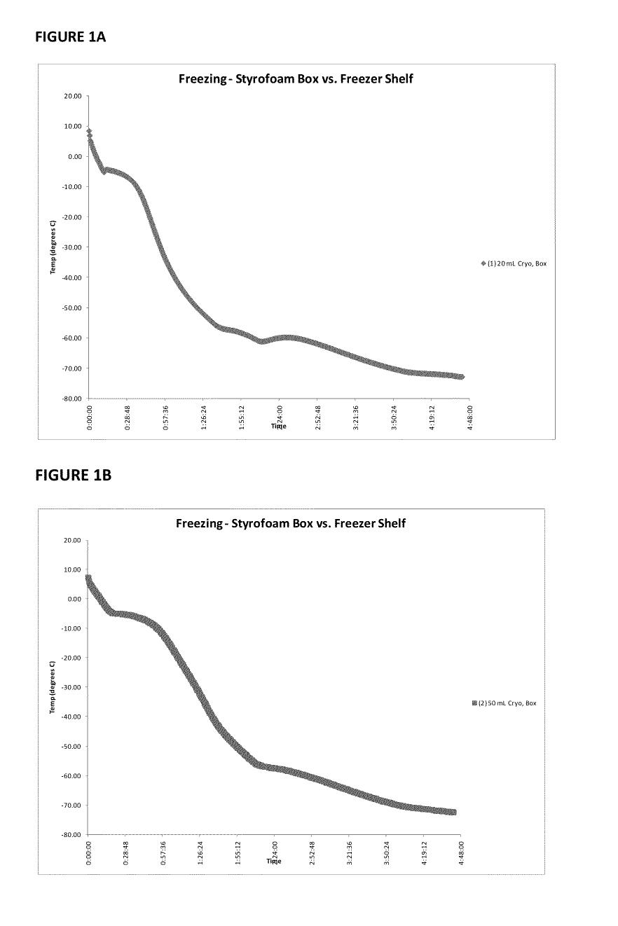

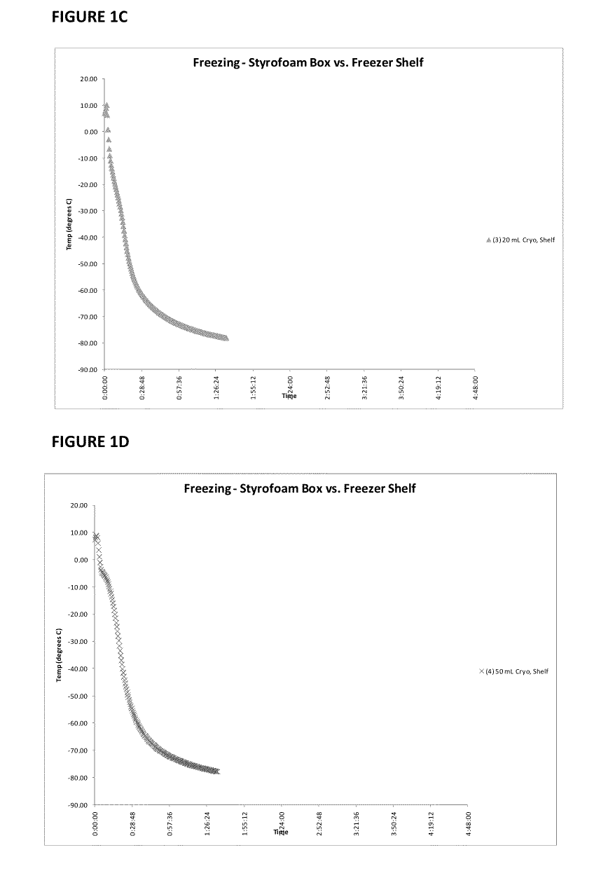

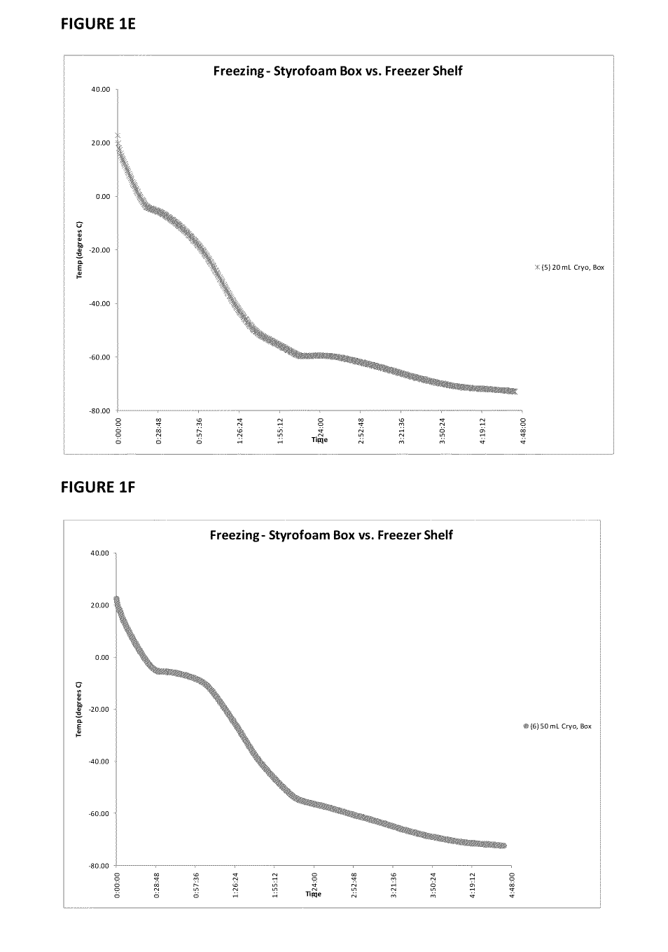

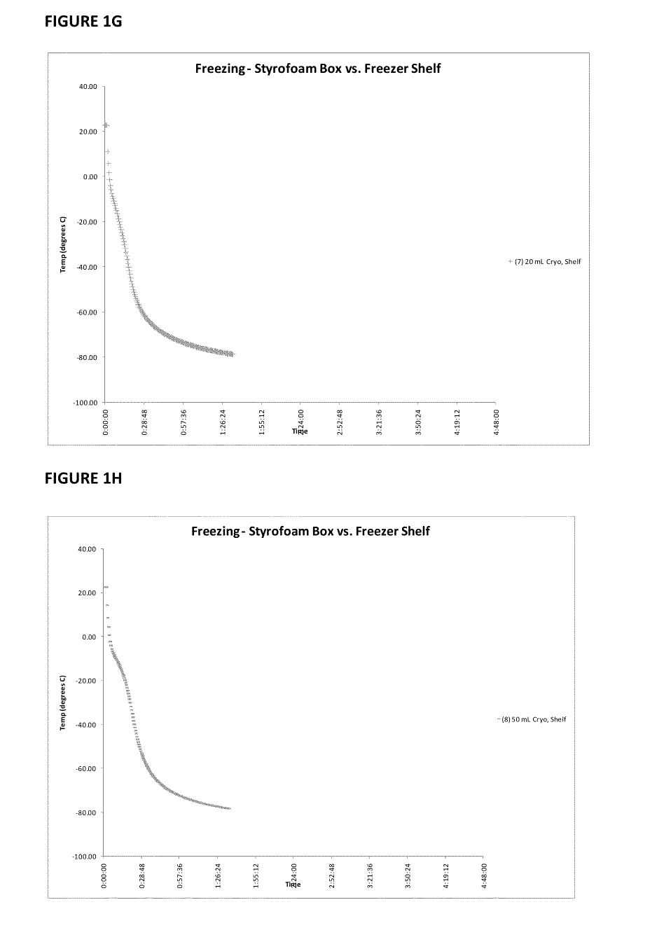

Temperature mapping experiments were performed to analyze the temperature profiles of potential cryopreservation conditions for the membrane products. These results are illustrated in FIG. 1. Eight (8) FP-90 cryobags were filled with either 20 mL or 50 mL of cryopreservation solution, and temperature probes were placed inside each cryobag. The first set of parameters (conditions 1 through 4 of FIG. 1A through FIG. 1D, respectively) involved a 30-minute refrigeration (2-8° C.) step prior to freezing (−80° C.±5° C.). In addition, the analysis involved freezing of the cryobags either inside a Styrofoam container or on the freezer shelf. The second set of parameters (conditions 5 through 8 of FIG. 1E through FIG. 1H, respectively) involved direct freezing (−80° C.±5° C.) of the cryobags either inside a Styrofoam container or on the freezer shelf. The results indicated that condition 6 and condition 2 exhibited the most gradual temperature decreases. Gradual temperature decreases are typically desired in order to preserve cell viability. The difference between condition 6 and condition 2 was that condition 2 included a 30-minute refrigeration step. Therefore, the decrease in temperature from the start of freezing to −4° C., where latent heat evolution upon freezing occurs, was examined further. For condition 6, the rate of cooling was approximately −1° C./minute during this period. The rate of cooling for condition 2 was approximately −0.4° C./minute during the same timeframe. Therefore, condition 2 was selected for incorporation into a non-limiting cryopreservation process since slower rates of cooling are generally desired to maintain optimal cell viability.

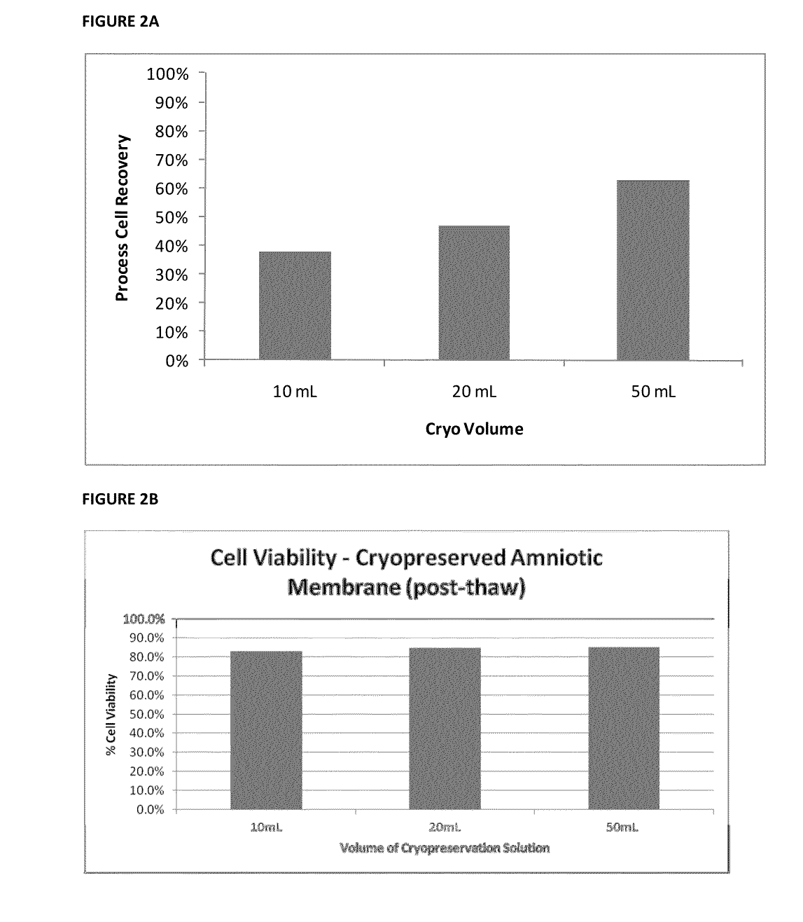

FIG. 2A depicts the effects of cryopreservation solution volume on process (cryopreservation) cell recovery for the amniotic membrane. FIG. 2B depicts the effects of cryopreservation solution volume on cell viability for the amniotic membrane.

Process (cryopreservation) cell recovery was calculated by comparing the number of viable cells before and after the cryopreservation process (as described previously herein). The intention was to identify cryopreservation conditions that provide maximum cell recovery. However, the utilized assay (trypan blue exclusion) requires taking tissue samples from different parts of the placenta, and since each tissue sample contains different cell counts, it is impossible to obtain an accurate cell recovery measurement. This explains the great range of process cell recovery (10%-410%) we have encountered. On the other hand, cell viability was calculated by comparing the number of live cells with the total number of cells in the same piece of tissue. Therefore, cell viability was used to optimize the parameters of the cryopreservation procedure.

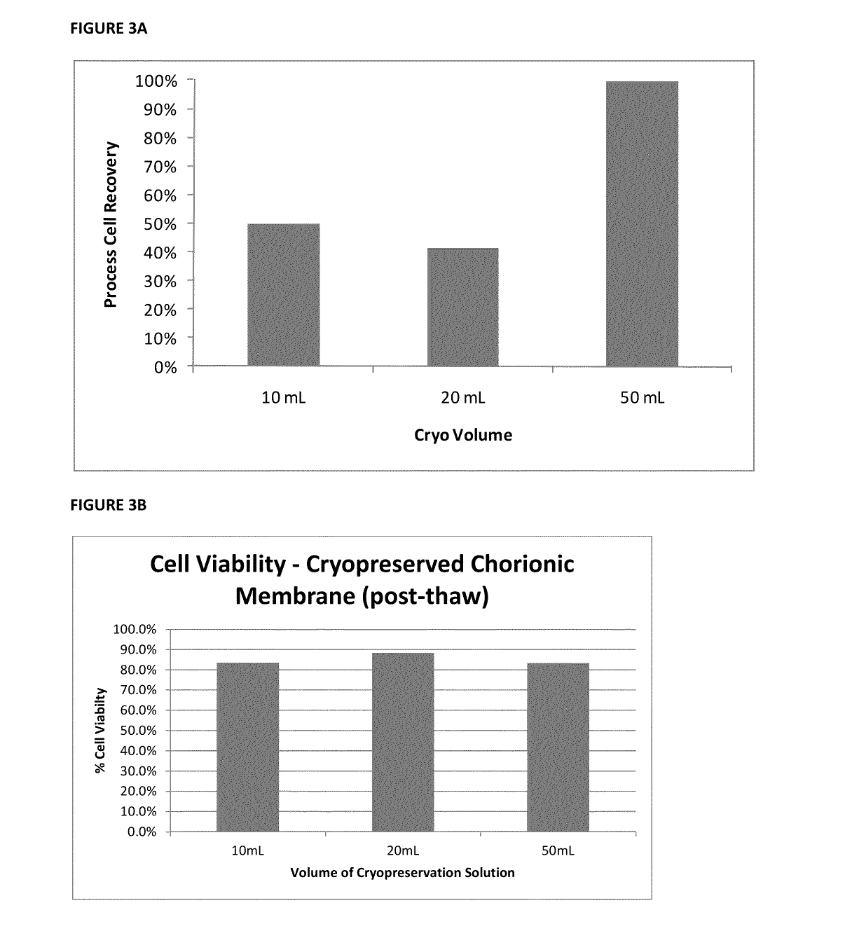

As depicted in FIG. 2B, the 50 mL volume of cryopreservation solution volume provided equivalent cell recovery compared to that of the 10 mL and 20 mL for a 5×5 placental membrane. These data indicate that 10 mL cryopreservation solution volume is sufficient to provide placental product with >70% cell viability according to the present technology. The same results were obtained for chorionic membrane as seen by FIGS. 3A and 3B.

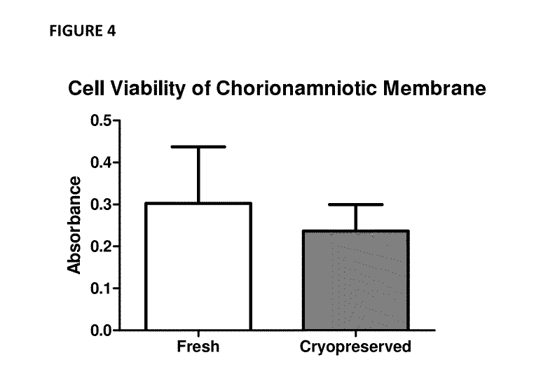

Experiments were conducted to evaluate whether the cryopreservation method could be applied to the preservation of the intact chorioamniotic membrane. A chorioamniotic membrane was cut out from the fresh placenta and incubated in antibiotics overnight. Trophoblast layer was then removed and the membranes were cut to 2 cm×2 cm pieces. Care was taken throughout the entire process so that the two membranes remained intact. Fresh and cryopreserved placental membranes were prepared and cell viability was measured using the MTT assay (Biotium 30006), a colorimetric assay to measure cellular viability. The mechanism of the assay is based on that fact that the metabolic reduction of the soluble tetrazolium salt to a blue formazan precipitate is dependent on the presence of viable cells with intact mitochondrial function. Samples were incubated in the MTT assay medium for 3-4 hours. At the end of the conversion period, samples were extracted for 1 hour at 37° C. in DMSO. At the end of the extraction period, 0.2 mL of each extract was transferred to a well of a 96-well plate. The absorbencies, which are proportional to cell viability, were read on a plate reader (Spectramax 340PC, Molecular Devices) at 570 nm with the DMSO extraction buffer as a blank. FIG. 4 shows the MTT values obtained from fresh and thawed cryopreserved samples. As indicated from the data, cell viability did not show significant difference between fresh and post-thaw cryopreserved chorioamniotic samples.

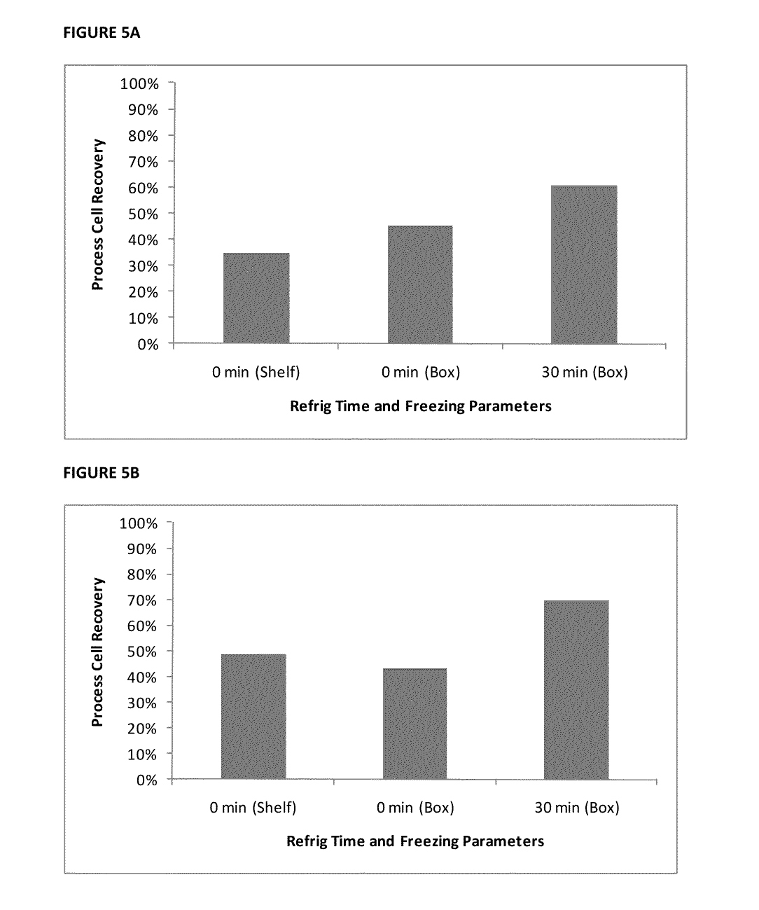

Experiments were conducted to evaluate different potential freezing conditions to maximize cell recovery after the cryopreservation process. FIG. 5A (amniotic membrane) and FIG. 5B (chorionic membranes) depict these results, showing the effects of refrigeration time and freezing parameters on process (cryopreservation) cell recovery for the chorionic membrane. Three conditions were analyzed. These conditions were also linked to the temperature mapping studies. The first condition involved directly freezing the product unit on a shelf within the freezer (−80° C.±5° C.). The second condition also contained a direct freeze, but the product unit was placed into a Styrofoam container within the freezer. The third condition included a refrigeration (about 2° C. to about 78° C.) period of 30 minutes prior to the freezing step. For the amniotic membrane, 3 placenta lots were evaluated. Two (2) placenta lots were analyzed for the chorionic membrane. Results indicated that the third condition was optimal for both membrane types.

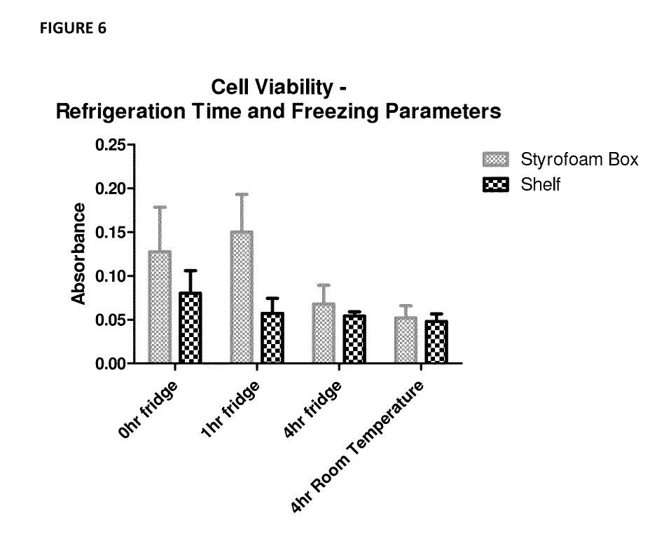

The effect of refrigeration time and freezing parameters on cell viability of amniotic membrane was also examined. Cell viability was measured using MTT assay described above where absorbance is proportional to cell viability. Four temperature equilibration times were tested: Direct freezing (0 hr), equilibration in refrigerator (4° C.) for 1 hr or 4 hrs, and equilibration at room temperature for 4 hrs. For each condition, freezing product directly on a freezer shelf or in a Styrofoam Box was also examined. FIG. 6 depicts the results of the study and shows that the highest cell viability was obtained when amniotic membranes were stored at 4° C. for up to 1 hour and then frozen within a Styrofoam box. This condition was chosen for further development as it provided the greatest processed cell recovery and cell viability.

All of the cryopreservation parameters that were assessed for the amniotic and chorionic membranes are summarized in Table 4 and Table 5.

The evaluation of cell viabilities from these experiments resulted in the selection of the final parameters for the manufacturing process. In addition, all average cell viability values were ≧70%.

TABLE 4
Post thaw cells per cm² and cell viability for the amniotic membrane.

| | | | | % Process | |
|---|---|---|---|---|---|
| | | | | Cell | |
| | | Viable | | Recovery | |
| | | Cells/ | % Cell | ((Proc./Fresh) * | |
| | | cm² | Viability | 100) | |
| | Condition | (Average ± | (Average ± | (Average ± | Comments/ |
| Parameter | Tested | SD) | SD) | SD) | Conclusions |
| Refrigeration | 30 min | 52,173 ± | 83.1% ± | 63.7% ± | No significant |
| time interval | | 39,750 | 4.5% | 21.4% | difference found in |
| | 60 min | 71,033 ± | 85.0% ± | 66.5% ± | process cell |
| | | 66,525 | 3.9% | 29.3% | recovery. A 30-60 min |
| | | | | | range was |
| | | | | | established. |
| Thawing | 37° C. ± | 48,524 ± | 83.3% ± | 64.0% ± | No significant |
| temperature | 2° C. water | 27,804 | 1.7% | 34.4% | difference found in |
| | bath | | | | process cell |
| | Room | 57,721 ± | 83.5% ± | 64.3% ± | recovery. The room |
| | temp | 49,271 | 4.9% | 19.0% | temp condition was |
| | water bath | | | | selected for logistical |
| | | | | | reasons. |
| Post-thaw | 1-15 min | 50,873 ± | 83.1% ± | 65.0% ± | No significant |
| stability in | | 38,969 | 3.9% | 24.2% | difference found in |
| saline | 1 hr | 76,667 ± | 85.1% ± | 61.0% ± | process cell |
| | | 66,565 | 6.2% | 14.3% | recovery. |
| | | | | | Membranes can be |
| | | | | | held in saline for up |
| | | | | | to 1 hr. |
| Tissue size | 5 cm × 5 cm | 58,431 ± | 83.3% ± | 62.8% ± | No decrease in |
| | | 47,603 | 4.5% | 21.7% | process cell |
| | | | | | recovery from the 5 cm × |
| | | | | | 5 cm product to |
| | | | | | the 2 cm × 2 cm |
| | | | | | product. Both sizes |
| | | | | | were acceptable for |
| | | | | | use. |

Notes:
cm = centimeter;
min = minutes;
temp = temperature;
hr = hour,
SD = standard deviation.

TABLE 5
Post thaw cells per cm² and cell viability for the chorionic membrane.

| | | | | % Process | |
|---|---|---|---|---|---|
| | | | | Cell | |
| | | Viable | | Recovery | |
| | | cells/ | | ((Proc./Fresh) * | |
| | | cm² | % Cell | 100) | |
| | Condition | (Avg ± | Viability | (Average ± | Comments/ |
| Parameter | Tested | SD) | (Avg ± SD) | SD) | Conclusions |
| Dispase | 30 min | 22,354 ± | 85.7% ± | 81.1% ± | No decrease in |
| treatment | | 9,505 | 5.1% | 32.4% | process cell |
| | 45 min | 27,125 ± | 90.6% ± | 172.6% ± | recovery for the |
| | | 7,963 | 2.2% | 101.2% | 45 min treatment. |
| | | | | | A 30-45 min |
| | | | | | range was |
| | | | | | established. |
| Refrigeration | 30 min | 23,815 ± | 86.8% ± | 102.2% ± | The process |
| time interval | | 9,681 | 5.2% | 68.8% | recovery value |
| | 60 min | 20,773 ± | 85.8% ± | 84.9% ± | was >80% for the |
| | | 7,356 | 4.7% | 14.4% | 60 min time |
| | | | | | interval. A 30-60 min |
| | | | | | range was |
| | | | | | established. |
| Thawing | 37° C. ± | 33,360 ± | 85.9% ± | 114.7% ± | No significant |
| temperature | 2° C. water | 8,497 | 4.0% | 38.1% | difference found |
| | bath | | | | in process cell |
| | Room | 21,298 ± | 86.8% ± | 96.3% ± | recovery. The |
| | temp | 8,189 | 5.3% | 67.2% | room temp |
| | water bath | | | | condition was |
| | | | | | selected for |
| | | | | | logistical reasons. |
| Holding | 1-15 min | 23,733 ± | 86.6% ± | 100.6% ± | No significant |
| period after | | 9,674 | 5.1% | 67.0% | difference found |
| transfer into | 1 hr | 20,550 ± | 87.0% ± | 91.4% ± | in process cell |
| saline | | 6,575 | 4.8% | 32.0% | recovery. |
| | | | | | Membranes can |
| | | | | | be held in saline |
| | | | | | for up to 1 hr. |
| Tissue size | 5 cm × 5 cm | 23,391 ± | 86.1% ± | 99.6% ± | No decrease in |
| | | 8,865 | 5.0% | 58.7% | process cell |
| | 2 cm × 2 cm | 23,036 ± | 88.4% ± | 98.7% ± | recovery from the |
| | | 11,362 | 5.0% | 81.3% | 5 cm × 5 cm |
| | | | | | product to the 2 cm × |
| | | | | | 2 cm |
| | | | | | product. Both |
| | | | | | sizes were |
| | | | | | acceptable for |
| | | | | | use. |

Notes:
cm = centimeter;
min = minutes;
temp = temperature;
hr = hour,
SD = standard deviation.

These data are consistent with certain embodiments of the present technology that provide a placental product comprising an amniotic membrane containing about 40,000 to about 90,000 or to about 260,000 cells/cm².

### Example 8

### Stability of Cryopreserved Amniotic Membrane

One unique aspect of the present technology is that the cryopreservation method is able to preserve cell viability for prolonged periods of time. In order to test whether cell viability would be maintained after two years of storage, three lots of amniotic membranes were manufacturing using the final manufacturing and cryopreservation process. Cell viability was determined using the quantitative method described in example 6 at Time 0 (1 week in storage), 12 months, 24 months and 25 months after storage. Table 6 demonstrates that after prolonged storage and subsequent thawing, cell viability remained above 70% for all lots at all time points.

TABLE 6
Cell Viability of Cryopreserved Amniotic Membranes
stored frozen for 25 months.

| | Time 0 | | | |
|---|---|---|---|---|
| Lot Number | (Initial Testing) | 12 Month | 24 Month | 25 Month |
| A11070 | 91.3% | 91.0% | 90.2% | 90.7% |
| A11081 | 78.6% | 82.3% | 87.8% | 92.4% |
| A11083 | 86.3% | 91.1% | 92.8% | 91.6% |

### Example 9

### Qualitative Evaluation of Cell Viability by Tissue Staining

The amniotic and chorionic membranes were stained using a LIVE/DEAD® Viability/Cytotoxicity kit (Molecular Probes Inc., Eugene, Oreg.) to qualitatively assess cell viability. Staining was performed as per the manufacturer's protocol. Membrane segments of approximately 0.5 cm×0.5 cm were used. Evaluation of stained membranes was performed using a fluorescent microscope. An intense uniform green fluorescence indicated the presence of live cells, and a bright red fluorescence indicated the presence of dead cells. Images of fresh amniotic and chorionic membranes as well as cryopreserved amniotic and chorionic membranes demonstrated that the manufacturing process did not alter the phenotypic characteristics of the membranes post thaw.

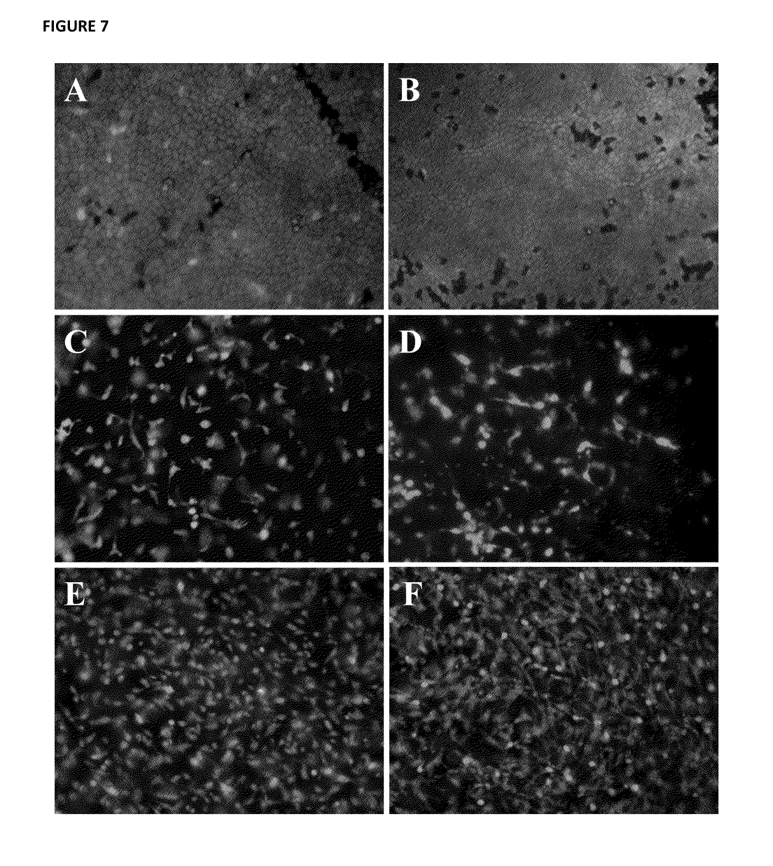

FIG. 7 contains representative images of the epithelial layer of fresh amniotic membrane (A), epithelial layer of cryopreserved amniotic membrane (B), stromal layer of fresh amniotic membrane (C), stromal layer of cryopreserved amniotic membrane (D), fresh chorionic membrane (E), and cryopreserved chorionic membrane (F). Live cells are green, and dead cells are red. These images demonstrated that the manufacturing process did not alter the phenotypic characteristics of the membranes and the proportion of viable cell types (epithelial and stromal cells) in the membranes post thaw.

### Example 10

### Exemplary Manufacturing Process Removes the Immunogenic Elements from the Placental Membranes

One unique feature of the human amnion and chorion is the absence of fetal blood vessels that prevent mobilization of leukocytes from fetal circulation. On the fetal side, macrophages resident in the chorioamniotic mesodermal layer represent the only population of immune cells. Thus, fetal macrophages present in the amnion and chorion are the major source of tissue immunogenicity. However, the number of macrophages in amnion is significantly lower than chorion (Magatti et al, Stem Cells, 2008, 26: 182), and this explains the low immunogenicity of amnion and the ability to use it across HLA barriers without matching between the donor and recipient (Akle et al, Lancet, 1981, 8254:1003; Ucakhan et al., Cornea, 2002, 21:169). In contrast, the chorion is considered immunogenic. In a study where the amnion was used together with the chorion for plastic repair of conjunctival defects, the success rate was low (De Roth Arch Ophthalmol, 1940, 23: 522). Without being bound by theory, the present inventors believe that removal of the maternal trophoblasts and CD14+ fetal macrophages, among other immunogenic cell types, from placental membranes prevents activation of lymphocytes in vitro. Removal of the maternal trophoblasts can be achieved by direct cleaning, whereas cryopreservation process described in this technology eliminates CD14+ cells.

Immunogenicity testing was used to characterize a placental product as a safe clinical therapeutic. Two bioassays (Mixed Lymphocyte Reaction (MLR) and Lipopolysaccharide (LPS)-induced Tumor Necrosis Factor (TNF)-α secretion) were used to test immunogenicity of placental products at different manufacturing steps.

### Example 10.1

### Mixed Lymphocyte Reaction (MLR)

An MLR is a widely used type of in vitro assay to test cell and tissue immunogenicity. The assay is based on the ability of immune cells (responders) derived from one individual to recognize allogeneic Human Leukocyte Antigen (HLA) and other antigenic molecules expressed on the surface of allogeneic cells and tissues (stimulators) derived from another individual when mixed together in a well of an experimental tissue culture plate. The response of immune cells to stimulation by allogeneic cells and tissues can be measured using a variety of methods such as secretion of particular cytokines (e.g., Interleukin (IL-2), expression of certain receptors (e.g., IL-2R), or cell proliferation, all of which are characteristics of activated immune cells.

Placental tissue samples representing different steps of the presently disclosed manufacturing process were used for immunogenicity testing. These samples included amnion with choriotrophoblast (ACT) as a starting material, separated choriotrophoblast (CT), chorion (CM), trophoblast (T), amnion (AM), and amnion and chorion (A/C) Both freshly purified and cryopreserved (final products) tissues were tested.

For the MLR assay, cells from placental tissues were isolated using 280 U/mL of collagenase type II (Worthington, Cat No. 4202). Tissues were treated with enzyme for 60-90 min at 37° C.±2° C., and the resulting cell suspension was filtered through a 100 μm filter to remove tissue debris. Single cell suspensions were then centrifuged using a Beckman, TJ-6 at 2000 rpm for 10 min and washed twice with DPBS. Supernatant was discarded after each wash, and cells were resuspended in 2 mL of DMEM (Invitrogen, Cat No. 11885) and evaluated for cell number and cell viability by counting cells in the presence of Trypan blue dye (Invitrogen, Cat No. 15250-061). Placental-derived cells were mixed with allogeneic hPBMCs at a 1:5 ratio in 24-well culture plates in DMEM supplemented with 5% fetal bovine serum (FBS) and incubated for 4 days in the incubator containing 5% CO₂, 95% humidity at 37° C.±2° C. Human Peripheral Blood Mononuclear Cells (hPBMCs) alone were used as a negative control, and a mixture of two sets of hPBMCs derived from two different donors was used as a positive MLR control. After 4 days of incubation, cells were collected from wells, lysed using a lysis buffer (Sigma, Cat No. C2978) supplemented with protease inhibitor cocktail (Roche, Cat No. 11836153001), and IL-2Rα was measured in cell lysates using the IL-2Rα ELISA kit (R&D Systems, Cat No. SR2A00) according to the manufacturer's protocol.

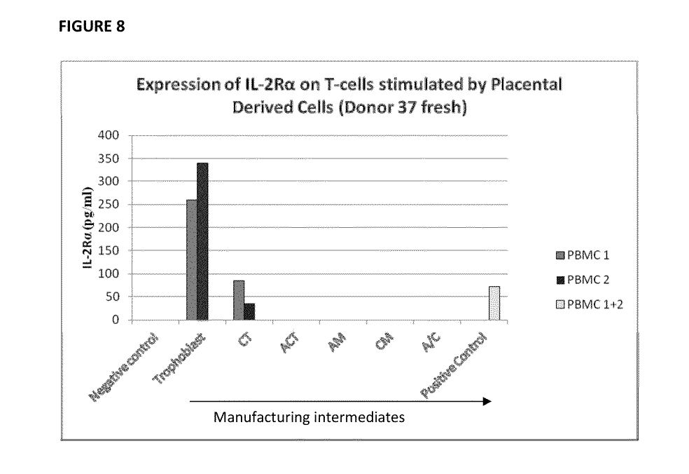

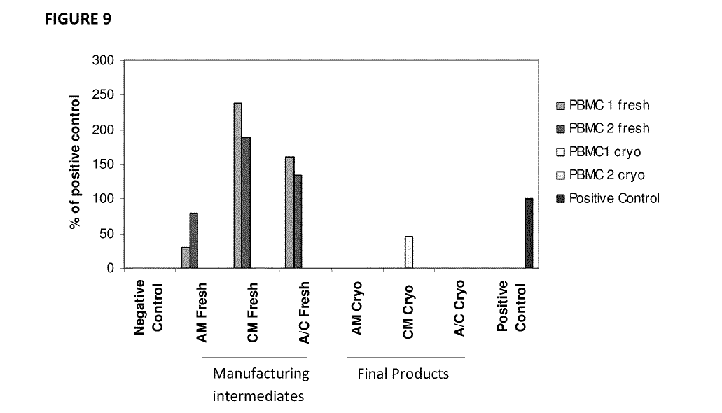

The level of IL-2Rα is a measure of activation of T-cells in response to immunogenic molecules expressed by allogeneic cells. Results presented in FIGS. 8 and 9 demonstrate a method of manufacture of placental membranes, resulting in low immunogenicity of the final products.

FIG. 8 demonstrates the manufacturing process serially reduces immunogenicity of the placental product. Samples representing different steps of the manufacturing process were co-cultured with hPBMCs for 4 days. IL-2Rα was measured in cell lysates as a marker of T-cell activation. Negative control shows a basal level of immune cell activation: PBMCs derived from one donor were cultured alone. Positive control: a mixture of PBMCs derived from 2 different donors.

FIG. 9 demonstrates selective depletion of immunogenicity results from the present cryopreservation process of producing the present placental products, as evidenced by the significant decrease in immunogenicity upon cryopreservation.

### Example 10.2

### LPS-Induced TNF-α Secretion by Placental Membrane Cells

As described herein, fetal macrophages present in the amnion and chorion are a major source of tissue immunogenicity. Without being bound by theory, the present inventors believe that removal of CD14+ fetal macrophages from placental membrane prevents activation of lymphocytes and decreases the level of inflammatory cytokine secretion and tissue immunogenicity. Macrophages in fetal placental membranes respond to bacterial LPS by secretion of inflammatory cytokines such as TNF-α. Therefore, secretion of TNF-α in response to LPS is used here to characterize tissue immunogenicity of placental membranes at each critical manufacturing step. Samples from each manufacturing step included trophoblast (T), amnion with chorio trophoblast (ACT), choriotrophoblast (CT), chorion (CM), and amnion (AM).

Pieces of placental membranes (2 cm×2 cm) representing intermediates and final products were placed in tissue culture medium and exposed to bacterial LPS (1 μg/mL) for 20-24 hr. Tissue culture supernatants were then collected and tested for the presence of TNF-α using a TNF-α ELISA kit (R&D Systems) according to the manufacturer's protocol. Human hPBMCs (SeraCare) known to contain monocytes responding to LPS by secretion of high levels of TNF-α were used as a positive control. hPBMCs and placental tissues without LPS were also included as controls in the analysis. In this assay, TNF-α detected in the culture medium from greater than about 70 pg/cm² (corresponding to 280 pg/mL) for both spontaneous and LPS-induced TNF-α secretion was considered immunogenic (Fortunato, et al. 1996).

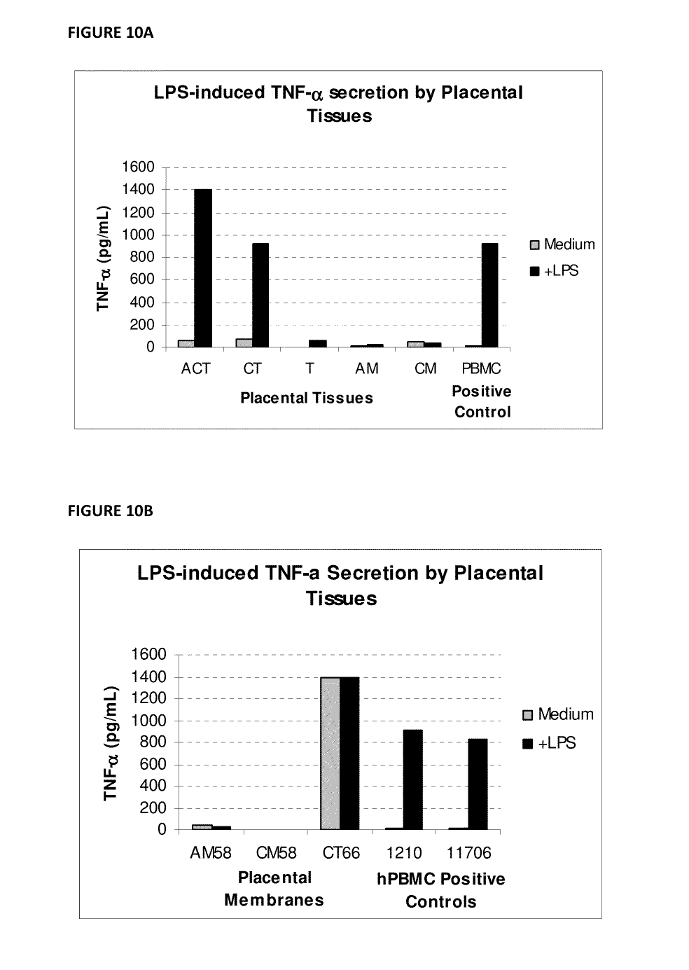

As depicted in FIGS. 10A and 10B, the manufacturing process serially reduces immunogenicity of the placental product. AM and CM had only 23.5 and 40 pg/ml TNF-α secretion as compared to ACT and CT at 1397.1 and 917.2 pg/ml, respectively. Tissues cultured in medium without LPS show the basal level of TNF-α secretion. PBMCs, which are known to secrete high levels of TNF, were used as a positive control.

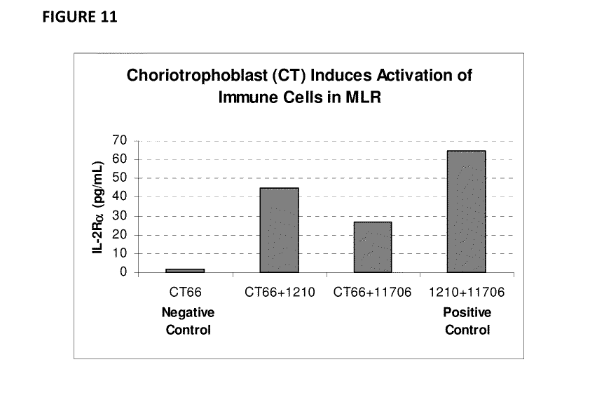

Choriotrophoblast membranes (CT) include the chorion membrane with an intact trophoblast layer. CT membrane which secreted high levels of TNF-α, was tested in MLR against two different PBMC donors (FIG. 11). CT cells were cultured with PBMCs for 4 days. IL-2Rα was measured in cell lysates as a marker of T-cell activation. Positive control: a mixture of PBMCs derived from 2 different donors. Results of this assay, as seen in FIG. 11, showed a correlation with the MLR data: tissues that produce high levels of TNF-α in response to LPS are immunogenic in the MLR assay

In conclusion, the low levels of TNF-α and the absence of the response to LPS by AM and CM indicates the exemplary cryopreservation method described in the current technology eliminates viable functional macrophages from the amniotic and chorionic membranes, which ensures the safety of such an allogeneic product.

### Characterization of the Cells Present in Placental Membranes

### Example 11

### Analysis of Placental Cells by Fluorescence-Activated Cell Sorting (FACS)

Knowing the cellular composition of amnion and chorionic membranes is important for developing a thorough understanding of potential functional roles in wound healing and immunogenicity. Previous reports demonstrated that both amnion and chorion contains multiple cell types. In addition to stromal cells that were identified for both the amnion and the chorion, amnion also contains epithelial cells. Although there are no fetal blood vessels within either the amniotic or chorionic membranes, both membranes comprise resident fetal macrophages. The close proximity to maternal blood circulation and decidua provide a potential source of immunogenic cells (maternal leukocytes and trophoblast cells) and therefore are a potential source of immunogenicity. To investigate the cellular composition of the amnion and chorion, Fluorescence-activated cell sorting (FACS) analysis was performed. The data demonstrated the presence of stromal cells in addition to fetal epithelial cells for amniotic membrane and stromal cells for chorionic membrane. One unique characteristic of the presently disclosed placental products is the presence of MSCs, which have been shown to be one of three types of cells (in addition to epithelial cells and fibroblasts) that are important for wound healing.

### Example 11.1

### FACS Procedure: Single Cell Suspension Preparation

Purified amnion and chorionic membranes were used for cellular phenotypic analysis via FACS. Cells from amnion and chorion were isolated using 280 U/mL collagenase type II (Worthington, Cat No. 4202). Tissues were treated with enzyme for 60-90 min at 37° C.±2° C., and the resulting cell suspension was filtered through a 100 μm filter to remove tissue debris. Single cell suspensions were then centrifuged using a Beckman TJ-6 at 2000 rpm for 10 min and washed twice with DPBS. Supernatant was discarded after each wash, and cells were resuspended in 2 mL of FACS staining buffer (DPBS+0.09% NaN₃+1% FBS).

### Example 11.2

### Immunolabeling Cells for Specific Cellular Markers

Once the single cell suspension was prepared according to Example 11.1, a minimum of 1×10⁵ cells in 100 μL of FACS staining buffer was treated with antibodies labeled with fluorescent dye. Table 7 provides descriptions of the antibodies used. For cell surface markers, cells were incubated for 30 min at room temperature in the dark with antibodies followed by washing twice with FACS staining buffer by centrifugation at 1300 rpm for 5 min using a Beckman TJ-6 centrifuge. Cells were then resuspended in 400 μl_of FACS staining buffer and analyzed using a BD FACSCalibur flow cytometer. To assess cell viability, 10 μL of 7-AAD (7-amino-actinomycin D) regent (BD, Cat No. 559925) was added just after the initial FACS analysis and analyzed again. For intracellular staining, cells were permeabilized and labeled following the manufacturer's recommendations (BD Cytofix/Cytoperm, Cat No. 554714) and analyzed using a BD FACSCalibur flow cytometer.

TABLE 7
Description of reagents used for placental
cell characterization by FACS.

| Cell marker | | | |
|---|---|---|---|
| antibody and | | Cell marker | Cell marker |
| label type | Catalog # | type | specificity |
| CD105-PE | Caltag | Cell surface | MSC marker |
| | MHCD10504 | | |
| CD166-PE | BD 559263 | Cell surface | MSC marker |
| CD73-PE | BD 550257 | Cell surface | MSC marker |
| CD90- | BD 550402 | Intracellular | MSC marker |
| unlabeled | | | |
| CD45-PE | BD 555483 | Cell surface | Pan Hematopoetic cell |
| | | | marker |
| CD34-APC | BD 340667 | Cell surface | Hematopoetic marker |
| CD40-FITC | BD 556624 | Cell surface | Immune co- |
| | | | stimulatory marker |
| CD86-FITC | BD 557343 | Cell surface | Immune co- |
| | | | stimulatory marker |
| CD14-PE | BD 555398 | Cell surface | Monocyte marker |
| HLA-DR-PE | BD 556644 | Cell surface | HLA class II specific |
| | | | for antigen-presenting |
| | | | cells |
| Cytokeratin 7- | Dako M7018 | Intracellular | Trophoblast marker |
| unlabeled | | | |
| CD19-APC | BD561742 | Cell surface | B-lymphocyte marker |
| CD41a-PE | BD 555467 | Cell surface | Platelet marker |
| CD49a- | Pierce | Cell surface | Integrin α1 |
| unlabeled | MA49A0 | | |
| Rabbit anti- | Dako F0261 | Intracellular | Secondary antibody |
| mouse FITC | | | |
| IgG1 isotype- | Dako X0931 | Intracellular | Isotype control |
| unlabeled | | | |
| IgG1 isotype- | BD555748 | Cell surface | Isotype control |
| FITC | | | |
| IgG1 isotype-PE | BD 559320 | Cell surface | Isotype control |
| IgG2a isotype- | BD 555574 | Cell surface | Isotype control |
| PE | | | |

### Example 12

### Phenotypic Analysis of Placental Cells

FACS analysis of single cell suspensions of both amnion and chorion membranes demonstrates that both membranes contain cells expressing markers specific for mesenchymal stem cells such as CD105 and CD166 (refer to Table 7), implicating the presence of MSCs. In addition, very few placental macrophages expressing CD14 were detected. Some immunogenic markers, which are more likely expressed on CD14+ placental macrophages, were detected, but the ranges of these markers are very wide. These ranges can be explained by: 1) high variability in cell number between placenta donors; and 2) technical issues, which include the presence of high and variable cellular and tissue debris in the cellular suspension. Although debris can be gated out, debris particles that are comparable with cells by size will affect the accuracy of the calculated % for each tested marker. Table 8 shows the characterization of the cellular composition of placental membranes based on selective CD markers. In addition, Table 9 provides a FACS analysis of cells isolated from the amniotic and chorionic membranes that were cultured in 10% FBS in DMEM at 37° C.±2° C. until confluency (passage 0 cells).

These data demonstrated that cells derived from amniotic and chorionic membranes retained a phenotype similar to MSCs after culturing. In conclusion, the presence of stromal cells in placental tissues was confirmed by FACS analysis.

These data are consistent with certain embodiments of the present technology that provide a placental product comprising an amniotic membrane containing MSCs.

TABLE 8
Characterization of the cellular composition of
placental membranes based on selective CD markers.

| | | Amnion | Chorion |
|---|---|---|---|
| | Marker | (% range) | (% range) |
| MSC Markers | CD105 | 72.1-88.2 | 6.4-78.5 |
| | CD166 | 17.3-58.0 | 4.8-51.5 |
| Hematopoietic Cell | CD14 | 6.93-10.5 | 0.9-6.1 |
| Markers | CD45 | 4.4-9.9 | 4.6-14.7 |
| Immune co- | HLA-DR | 0-5.6 | 0-14.7 |
| stimulatory markers | CD86 | 24.3-49.6 | 4.9-22.5 |
| | CD40 | 7.0-68.7 | 2-5.8 |
| Trophoblast marker | Cytokeratin-7 | 1.36-4.66 | 2.71-23.07 |

TABLE 9
FACS analysis of cultured cells (passage 0)
from placenta lot D16.

| Cell Surface Marker | Amnion (%) | Chorion (%) |
|---|---|---|
| CD45 | 2.18 | 0.53 |
| CD166 | 92.77 | 82.62 |
| CD105 | 83.02 | 86.73 |
| CD49a | 92.28 | 92.26 |
| CD73 | 89.57 | 94.57 |
| CD41a | −0.03 | −0.05 |
| CD34 | −0.23 | −0.25 |
| HLA-DR | −0.23 | −0.19 |
| CD19 | −0.19 | −0.22 |
| CD14 | −0.25 | −0.27 |
| CD90 | 99.12 | 98.00 |

### Example 13

### Adherence of Cells Derived from Placental Products

In addition to the presence of specific cellular markers, a cell can be classified as an MSC if it shows plastic adherence properties under normal culture conditions and has a fibroblast-like morphology. Cells were isolated from the amniotic and chorionic membrane products as described in this technology, plated into MSC media and cultured at 37° C.±2° C. until they reach confluency. Their ability to adhere to the plastic culture dishes was then evaluated.

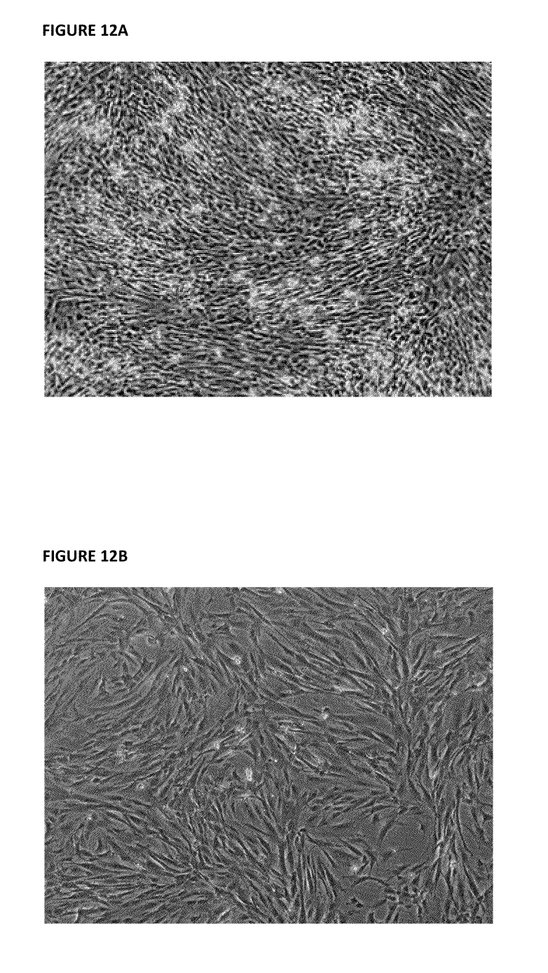

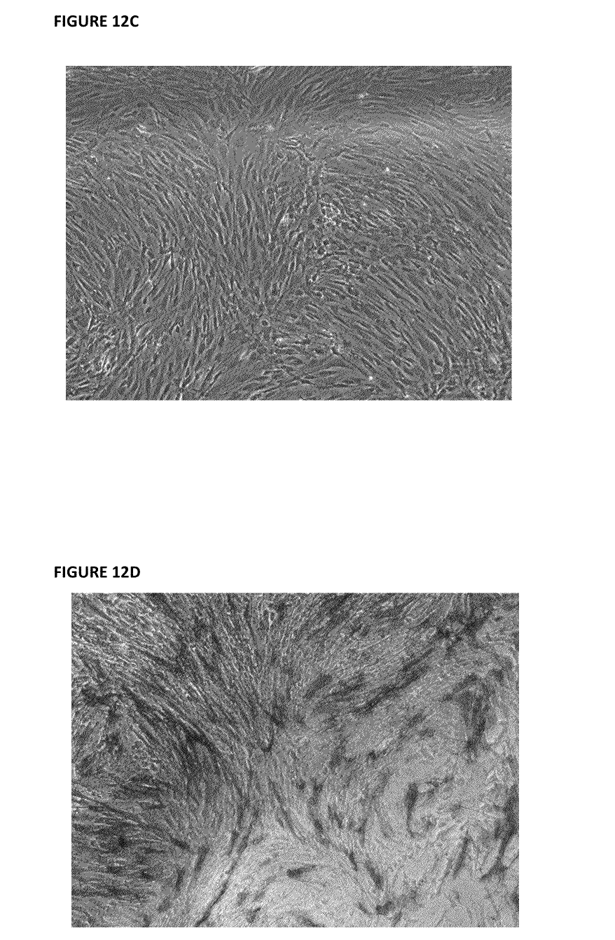

FIG. 12 demonstrates plastic adherence of cells isolated and cultured from amniotic (FIG. 12A) and chorionic (FIG. 12B), which is similar to MSCs isolated and expanded from human bone marrow aspirate (FIG. 12C). These data show that cells derived from amniotic and chorionic membranes retain a phenotype similar to MSCs.

In addition, MSCs are also defined by their ability to differentiate into different connective tissue types. Thus, the ability of placental-derived cells to undergo osteogenic differentiation was tested. Placental cells were isolated and cultured at 37° C.±2° C. until they reach confluency. Then, osteogenic medium was added to the culture and expression of alkaline phosphatase was measured. Alkaline phosphatase is an enzyme involved in the mineralization of bone and is a well known osteogenic marker. FIG. 12D shows that several cells are stained with a purple dye which represents alkaline phosphatase expression. This demonstrates that MSCs present in placental membranes retain their differentiation potential.

### Therapeutic Factors

### Example 14

### Cryopreservation does not Compromise the Level of Therapeutic Factors in Placental Membranes

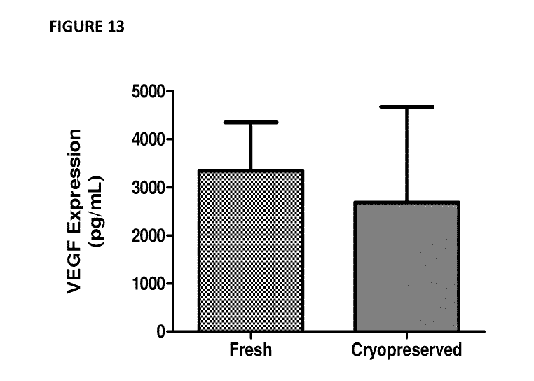

We first investigated whether cryopreservation process affects the level of growth factors that are important for wound healing. Vascular endothelial growth factor (VEGF) was chosen as it is critical for promoting angiogenesis. VEGF was extracted using an 8M guanidine hydrochloride (GuHCl) solution, bead homogenizer, and incubation with GuHCl solution for 24 hours. VEGF expression from fresh amniotic membrane and cryopreserved amniotic membrane product (as described by this present technology) was measured using Enzyme-Linked Immunosorbant Assay (ELISA) as per manufacturer's protocol. Results in FIG. 13 show that by cryopreserving the membrane using the procedures described in this present technology, there is no loss in the expression of the growth factor as compared to fresh membrane.

### Example 15

### Therapeutic Factors Analyses

The protein profiles of amniotic and chorionic membranes were investigated using a SearchLight Multiplex chemiluminescent array or ELISA. The presence of proteins in tissue membrane extracts and secreted by tissues in culture medium was investigated. Testing consisted of an analysis of proteins that are important for wound healing. The list of identified proteins is described in Table 10.

TABLE 10
List of selected Therapeutic Factors for analysis.

| Function | Therapeutic Factors |
|---|---|
| Angiogenesis | Angiotensin-2 (Ang-2), Fibroblast Growth |
| | Factor basic (bFGF), heparin-bound |
| | Epidermal Growth Factor (HB-EGF), EGF, |
| | Keratinocyte Growth Factor (KGF-also |
| | known as FGF-7), Platelet derived Growth |
| | Factors (PDGF) AA, AB and BB, Vascular |
| | Endothelial Growth Factor (VEGF), VEGF-C, |
| | Hepatocyte Growth Factor (HGF), Placental |
| | Growth Factor (PIGF), Pigment Epithelium |
| | Derived Factor (PEDF), Trombospondin-1 |
| | (TSP-1), TSP-2 |
| Re-epithelialization | Epidermal Growth Factor (EGF), |
| | Keratinocyte Growth Factor (KGF), |
| | Adiponectin (Acrp-30), Insulin Growth Factor |
| | 1 (IGF), Insulin-like growth factor binding |
| | protein (IGFBP 1, 2, 3), Transforming Growth |
| | Factor α (TGFα), TGF-β1, TGF-β2 |
| Anti-microbial | Neutrophil gelatinase-associated lipocalin |
| | (NGAL), Defensin |
| Chemoattractant | Stromal Cell Derived Factor 1 Beta (SDF- |
| | 1b), bFGF, EGF, KGF |
| Anti-Scarring | TGF-β3, Interferon 2α (IFN-2α) |
| Extracellular | Matrix Metalloproteinase 1 (MMP1), |
| Matrix Remodeling - | MMP2,3,7,8,9,10,13, Tissue Inhibitors of |
| Proteases and | MMPs (TIMP1 and 2), Alpha-2- |
| Protease Inhibitors | macroglobulin, Fibronectin |
| Immunoregulatory | Granulocyte Colony-Stimulating Factor (G- |
| | CSF), Interlaukin1 receptor antagonist (IL- |
| | 1RA), Leukemia Inhibitory Factor (LIF), |
| | Interferon 2α (IFN-2α), Placental Bone |
| | Morphogenetic Protein (PLAB) |

### Example 15.1

### Preparation of the Amniotic and Chorionic Membrane Samples for Therapeutic Factors Profiling

Amniotic and chorionic membranes were isolated and packaged at −80° C.±5° C. according to the manufacturing protocols disclosed herein in Examples 1 and 2. Packaged membranes were then thawed in a 37° C.±2° C. water bath and washed 3 times with DPBS. Membranes were cut into 8 cm² pieces. For tissue lysate samples, one 8 cm² piece of membrane was snap frozen in liquid nitrogen followed by pulverization using a mortar and pestle. Crushed tissue was transferred to a 1.5 mL microcentrifuge tube and 500 μL of Lysis buffer (Cell Signaling Technologies, Cat No. 9803) with protease inhibitor (Roche, Cat No. 11836153001) was added and incubated on ice for 30 min with frequent vortexing. Tissue lysate was then centrifuged at 16000 g for 10 min. The supernatant was collected and sent for protein array analysis by Aushon Biosystems. For supernatant samples, one 8 cm² piece of membrane was plated onto a well of a 12-well dish and 2 mL of DMEM+1% HSA+antibiotic/antimycotic were added and incubated at 37° C.±2° C. for 3, 7, or 14 days. After incubation, tissue and culture media were transferred to a 15 mL conical tube and centrifuged at 2000 rpm for 5 min. Culture supernatant was collected and sent for protein array analysis by Aushon Biosystems.

### Example 15.2

### Therapeutic Factors Present in Tissue Lysates

Placental product lysates were analyzed for the presence of proteins that are important in tissue repair. Table 11 depicts the biochemical profile of the lysates of exemplary placental tissue products of the present technology.

TABLE 11
Therapeutic factors present in Tissue Lysates of Exemplary
Placental Tissues (pg/mL).

| | AM75 lysate | AM78 lysate | CM75 lysate | CM78 lysate |
|---|---|---|---|---|
| Factor | pg/ml | pg/ml | pg/ml | pg/ml |
| hACRP30 | 50.8 | 1154.6 | 1213.7 | 225.3 |
| hAlpha2- | 1910.6 | 426191.6 | 8174.4 | 9968.6 |
| Macroglobulin | | | | |
| hEGF | 127.3 | 361.4 | 0.0 | 0.8 |
| hbFGF | 119.1 | 821.5 | 375.0 | 351.3 |
| hGCSF | 0.7 | 3.2 | 1.2 | 0.7 |
| hHBEGF | 127.5 | 168.0 | 15.4 | 84.5 |
| hHGF | 3943.7 | 15060.0 | 29979.6 | 50392.8 |
| hIGFBP1 | 5065.0 | 9456.6 | 934.0 | 1443.6 |
| hIGFBP2 | 12460.8 | 5569.7 | 135.9 | 134.6 |
| hIGFBP3 | 50115.7 | 41551.4 | 4571.5 | 11970.2 |
| hIL1ra | 3881.0 | 32296.9 | 5168.2 | 525.5 |
| hKGF | 1.4 | 8.8 | 3.1 | 1.5 |
| hLIF | 0.0 | 4.2 | 0.0 | 0.0 |
| hMMP1 | 9144.1 | 20641.2 | 2882.9 | 6582.3 |
| hMMP10 | 0.0 | 15.5 | 79.3 | 87.5 |
| hMMP2 | 2067.3 | 4061.9 | 949.5 | 748.8 |
| hMMP3 | 0.0 | 36.2 | 0.0 | 0.0 |
| hMMP7 | 5.1 | 11.4 | 4.5 | 9.1 |
| hMMP8 | 0.0 | 0.0 | 0.0 | 0.0 |
| hMMP9 | 92.2 | 2878.1 | 2676.2 | 1259.3 |
| hNGAL | 6900.1 | 6175.9 | 938.5 | 229.7 |
| hPDGFAA | 0.0 | 12.5 | 39.8 | 35.2 |
| hPDGFAB | 11.2 | 31.3 | 14.4 | 14.0 |
| hPDGFbb | 4.6 | 13.4 | 4.0 | 1.3 |
| hPEDF | 0.0 | 652.6 | 0.0 | 0.0 |
| hTIMP1 | 7958.1 | 35955.6 | 50712.3 | 17419.9 |
| hTIMP2 | 3821.8 | 7443.2 | 640.7 | 780.0 |
| hVEGF | 3.3 | 11.8 | 125.2 | 8.4 |
| hVEGFC | 46.5 | 150.0 | 123.5 | 51.7 |
| hVEGFD | 25.7 | 31.0 | 15.0 | 20.4 |

### Example 15.3

### Sustained Release of Therapeutic Factors Over a Period of 14 Days

Placental products of the present technology also demonstrate a durable effect, which is desirable for wound healing treatments. The extracellular matrix and presence of viable cells within the amniotic membrane described herein allow for a cocktail of proteins that are known to be important for wound healing to be present for at least 14 days. Amniotic membranes were thawed and plated onto tissue culture wells and incubated at 37° C.±2° C. for 3, 7, and 14 days. At each time point, a sample of the culture supernatant was collected and measured through protein array analysis as described in Example 15.1. Table 12 illustrates the level of various secreted factors in tissue culture supernatants of amniotic membrane lots at 3, 7 and 14 days as measured through protein array analysis.

TABLE 12
Levels of proteins secreted in amnion tissue culture
supernatants at different time points (pg/ml).

| Factor | Day 3 | Day 7 | Day 14 |
|---|---|---|---|
| hACRP30 | 548.03 | 766.73 | 319.75 |
| hAlpha2Macroglobulin | 69687.55 | 31764.00 | 48477.62 |
| hANG2 | 0.00 | 9.28 | 1.65 |
| hEGF | 3.06 | 2.51 | 6.15 |
| hbFGF | 40.80 | 85.46 | 41.1 |
| hFibronectin | 1932101.25 | 3506662.00 | 3120902 |
| hHBEGF | 41.78 | 80.50 | 53 |
| hHGF | 5358.09 | 9327.67 | 1631.35 |
| hIGFBP1 | 2654.57 | 6396.11 | 6364.6 |
| hIGFBP2 | 4379.76 | 23797.46 | 1552.15 |
| hIGFBP3 | 36030.52 | 107041.71 | 11218.55 |
| hIL1ra | 116593.20 | 675.09 | 95028.3 |
| hKGF | 7.29 | 13.86 | 3.05 |
| hMMP1 | 323249.53 | 1727765.60 | 77157.55 |
| hMMP10 | 14804.44 | 20557.91 | 5432.65 |
| hMMP13 | 92.92 | 408.17 | 140.95 |
| hMMP2 | 38420.90 | 322500.72 | 44233.15 |
| hMMP3 | 66413.54 | 283513.74 | 522.25 |
| hMMP7 | 128.51 | 147.65 | 93.35 |
| hMMP8 | 463.32 | 2109.21 | 192.45 |
| hMMP9 | 6139.53 | 25810.38 | 1384.45 |
| hNGAL | 15754.19 | 70419.63 | 3400.95 |
| hPDGFAA | 18.02 | 58.69 | 12.95 |
| hPDGFAB | 16.58 | 58.41 | 28.30 |
| hPDGFBB | 1.94 | 21.67 | 8.75 |
| hPEDF | 6793.74 | 21645.90 | 8060.3 |
| hSDF1b | 0.00 | 24.09 | 37.12 |
| hTGFa | 15.05 | 14.89 | 2 |
| hTGFb1 | 334.07 | 341.53 | 680.33 |
| hTGFb2 | 119.59 | 207.79 | 731.96 |
| hTIMP1 | 197743.23 | 437492.21 | 196491.9 |
| hTIMP2 | 4724.25 | 19970.76 | 6325 |
| hTSP1 | 0.00 | 0.00 | 157.5 |
| hTSP2 | 13820.61 | 59695.21 | 13988.65 |
| hVEGF | 44.98 | 57.45 | 7.40 |
| hVEGFC | 307.75 | 569.9 | 516.2 |

### Example 15.4

### Presence of Interferon 2α (IFN-2α) and Transforming Growth Factor-β3 (TGF-β3) in Amniotic Membrane

Interferon-α and TGF-β3 are cytokine/growth factor known to reduce fibrosis in various tissues. Clinically, IFN-2α and TGF-β3 have been suggested to modulate wound healing by the prevention of scar and contracture formation (Ishida, Kondo et al. 2004; Ferguson, Duncan et al. 2009). IFN-2α may serve a role to inhibit fibroblast proliferation, decrease collagen and fibronectin synthesis and fibroblast-mediated wound contracture (Wang, Crowston et al. 2007). Clinically, IFN-2α has been administered subcutaneously and shown to improve scar quality (Nedelec et al, Lab Clin Med 1995, 126:474). TGF-β3 regulates the deposition of extracellular matrix and has been shown to decrease scar formation when injected in rodent cutaneous wound models. Clinically, TGF-β3 has been shown to improve scar appearance when injected at the wound site (Occleston et al., J Biomater Sci Polym Ed 2008, 19:1047). TGF-β3 works as a TGF-β1 antagonist, modulating fibroblast-myofibroblast differentiation, and restricting profibrotic gene transcription (Chang, Kishimoto et al. 2014).

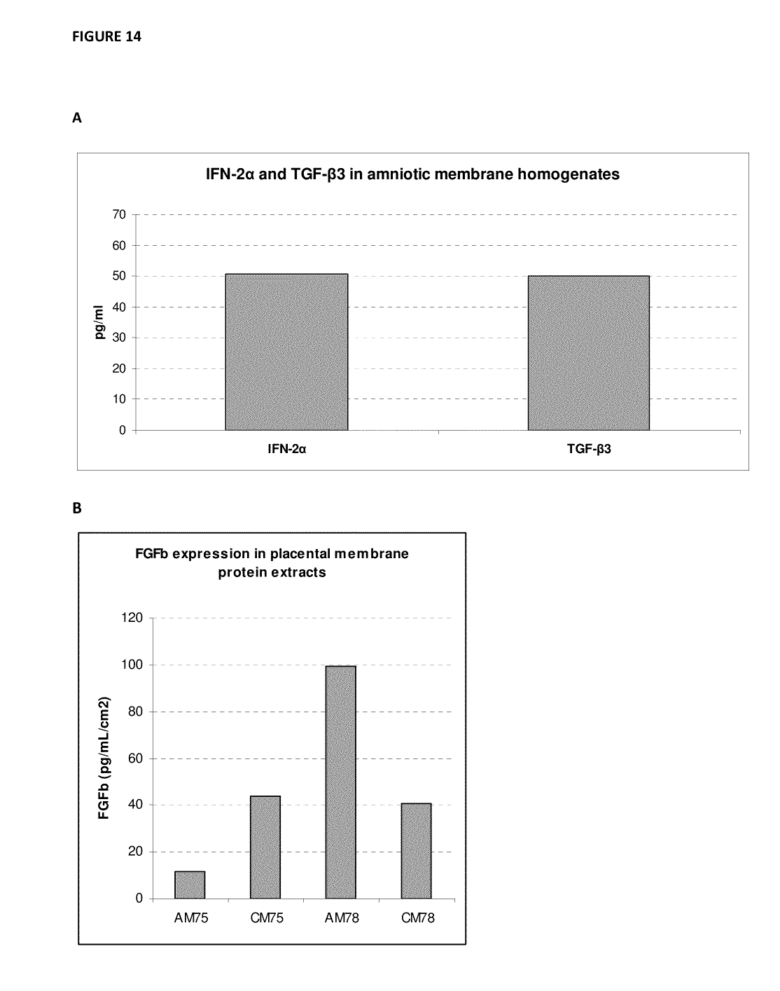

Placental products described in the present technology have been analyzed for the presence of IFN-2α and TGF-β3. Briefly, after thawing, the membranes were homogenized and centrifuged at 16,000 g to collect the resulting supernatants. Supernatants were analyzed on a commercially available ELISA kit from MabTech (IFN-2α) and R&D Systems (TGF-β3). FIG. 14 shows significant expression of IFN-2α (A) and TGF-β3 (B) in placental product homogenates.

### Example 15.5

### Presence of bFGF in Amniotic and Chorionic Membrane

bFGF modulates a variety of cellular processes including angiogenesis, tissue repair, and wound healing (Presta et al., 2005, Reuss et al., 2003, and Su et al., 2008). In wound healing models, bFGF has been shown to increase wound closure and enhance vessel formation at the site of the wound (Greenhalgh et al., 1990). Evaluation of proteins derived from amniotic and chorionic membranes prepared pursuant to the presently disclosed manufacturing process revealed that bFGF is one of the major factors in placental tissue protein extracts. FIG. 14 B depicts expression of bFGF by amniotic membranes (AM) and chorionic membranes (CM) from two donors detected during the protein profile evaluation of placental membranes.

### Example 15.6

### Presence of Placental Growth Factor PLGF (PLGF) and Insulin Growth Factor-1 (IGF-1) in Amniotic Membrane

Without being bound by theory, the inventors believe that efficacy of the present placental products for wound repair are due, in part, to the role of BMPs, IGF-1, and PIGF in the development and homeostasis of various tissues by regulating key cellular processes. BMP-2 and BMP-4 may stimulate differentiation of MSCs to osteoblasts in addition to promote cell growth; placental BMP or PLAB is a novel member of the BMP family that is suggested to mediate embryonic development. Insulin-like growth factor 1 (IGF-1) may promotes proliferation and differentiation of osteoprogenitor cells. Placental derived growth factor (PIGF) may acts as a mitogen for osteoblasts.

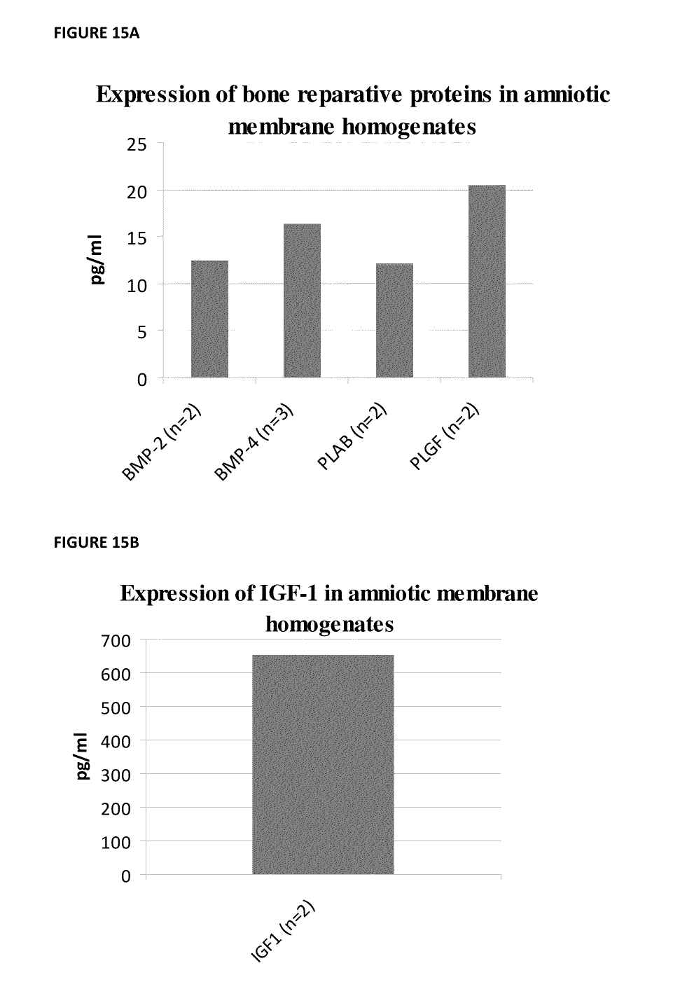

Placental products described in the present technology have been analyzed for the presence of tissue reparative proteins. Briefly, the thawed products were incubated in DMEM+10% FBS for 72 hrs. The membranes were then homogenized in a bead homogenizer with the culture media. The homogenates were centrifuged, and the supernatants were analyzed on commercially available ELISA kits from R&D Systems. FIG. 15 shows significant expression of BMP-2, BMP-4, PLAB, PIGF, and IGF-1 in several donors of amniotic membranes.

### Example 15.7

### Presence of α2-Macroglobulin in Amniotic Membranes

α2-macroglobulin is known as a plasma protein that inactivates proteinases from all 4 mechanistic classes, serine proteinases, cysteine proteinases, aspartic proteinases, and metalloproteinases. Another important function of this protein is to serve as a reservoir for cytokines and growth factors, examples of which include TGF, PDGF, and FGF. In the chronic wounds like diabetic ulcers or venous ulcers, the presence of high amount of proteases leads to rapid degradation of growth factors and delays in wound healing. Thus, the presence of α2-macroglobulin in products designed for chronic wound healing will be beneficial. Results of the protein array analysis showed that amniotic and chorionic membranes contain α2-macroglobulin (Table 13). Although these preliminary data show high variability between donors, the importance of this protein in wound healing prompted the additional evaluation of α2-macroglobulin in placental tissues using a single analyte ELISA instead of protein array, which is a useful tool to evaluate the presence of multiple proteins in one sample for profiling.

These data are consistent with certain embodiments of the present technology that provide a placental product comprising an amniotic membrane containing α2-macroglobulin.

TABLE 13
Expression of α2-macroglobulin in placental
tissue protein extracts.

| | α2-macroglobulin |
|---|---|
| Sample | (pg/mL/8 cm²) |
| AM75 | 7 |
| CM75 | 790 |
| AM78 | 53042 |
| CM78 | 1014 |

### Example 16

### Comparison of Therapeutic Factors in Exemplary Placental Tissues and Two Commercially Available Products

For comparison, the protein profiles of two commercially available products containing living cells, Dermagraft and Apligraf, were assayed as well using a SearchLight Multiplex chemiluminescent array.

### Example 16.1

### Protocol for Comparison of Therapeutic Factors of Exemplary Placental Tissues and Two Commercially Available Products

For testing Dermagraft, the membrane was thawed and washed according to the manufacturer's instructions. Dermagraft membrane was cut into 7.5 cm² pieces. For tissue lysates, one 7.5 cm² piece of membrane was snap frozen in liquid nitrogen followed by pulverization using a mortar and pestle. Crushed tissue was transferred to a 1.5 mL microcentrifuge tube and 500 μL of Lysis buffer (Cell Signaling Technologies, Cat No. 9803) with protease inhibitor (Roche, Cat No. 11836153001) was added and incubated on ice for 30 min with frequent vortexing. The sample was then centrifuged at 16000 g for 10 min. The supernatant was collected and sent for protein array analysis by Aushon Biosystems. For tissue culture, one 7.5 cm² piece of membrane was plated onto a well of a 12-well dish and 2 mL of DMEM+1% HSA+antibiotic/antimycotic were added and incubated at 37° C.±2° C. for 3, 7, or 14 days. After incubation, tissue and culture media were transferred to a 15 mL conical tube and centrifuged at 2000 rpm for 5 min. Culture supernatant was collected and sent for protein array analysis by Aushon Biosystems.

For testing Apligraf, the membrane was cut into 7.3 cm² pieces. For tissue lysates, one 7.3 cm² piece of membrane was snap frozen in liquid nitrogen followed by pulverization using a mortar and pestle. Crushed tissue was transferred to a 1.5 mL microcentrifuge tube and 500 μL of Lysis buffer (Cell Signaling Technologies, Cat No. 9803) with protease inhibitor (Roche, Cat No. 11836153001) was added and incubated on ice for 30 min with frequent vortexing. The sample was then centrifuged at 16000 g for 10 min. The supernatant was collected and sent for protein array analysis by Aushon Biosystems. For tissue culture, one 7.3 cm² piece of membrane was plated onto a well of a 12-well dish and 2 mL of DMEM+1% HSA+antibiotic/antimycotic were added and incubated at 37° C.±2° C. for 3, 7, or 14 days. After incubation, tissue and culture media were transferred to a 15 mL conical tube and centrifuged at 2000 rpm for 5 min. Culture supernatant was collected and sent for protein array analysis by Aushon Biosystems.

### Example 16.2

### Therapeutic Factors present in Day 3 Supernatants of Exemplary Placental Tissues and Commercially Available Products

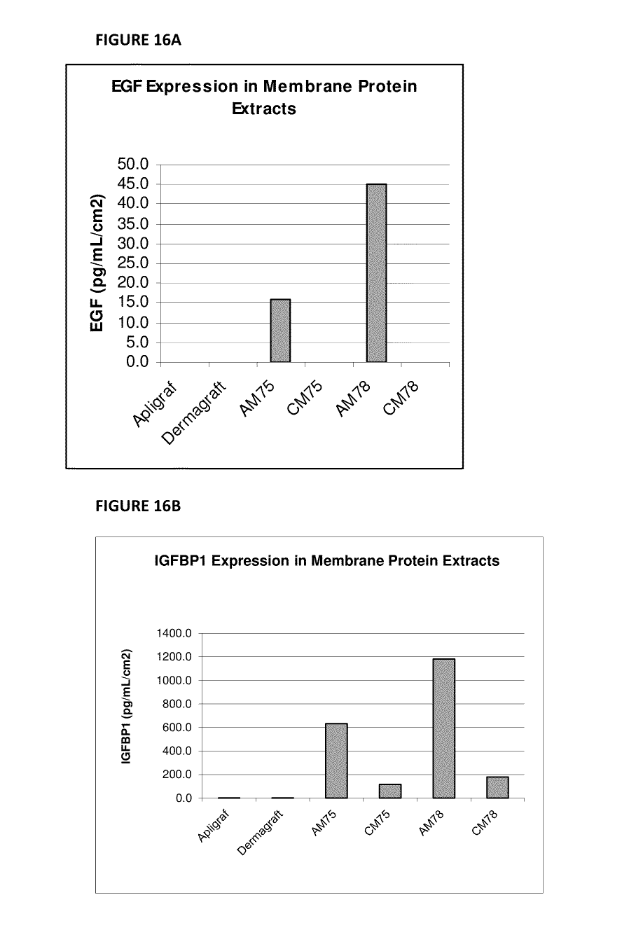

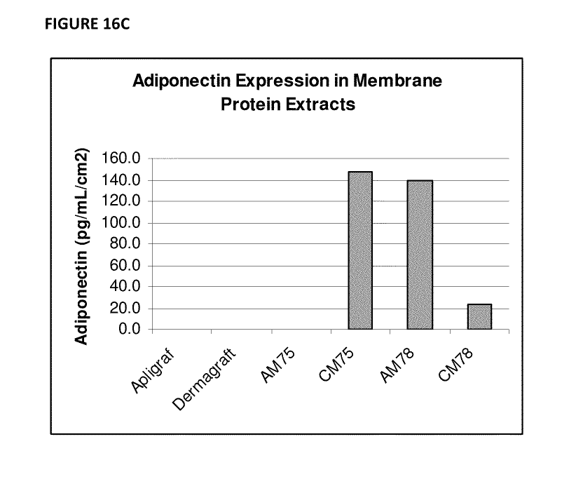

Protein array data analyses showed that the majority of selected testing factors (refer to Table 11) were expressed in amniotic membrane, chorionic membrane, Apligraf, and Dermagraft. Three proteins were identified as unique for the amniotic membrane and/or the chorionic membrane which are undetectable in Apligraf and Dermagraft. These proteins are EGF, IGFBP1, and Adiponectin. All three proteins are important for wound healing. FIG. 16 depicts expression of EGF (A), IGFBP1 (B), and Adiponectin (C) in amniotic (AM), chorionic membrane (CM) and commercially available products. AM75 and AM 78 are cryopreserved placental products of the present technology (e.g. cryopreserved), while CM75 and CM78 are cryopreserved chorionic membrane products. These proteins are believed by the inventors to facilitate the therapeutic efficacy of the present placental products for wound healing.

These data are consistent with certain embodiments of the present technology that provide a placental product comprising an amniotic membrane containing EGF, IGFBP1, and/or adiponectin.

Table 14 depicts the biochemical profile of the supernatants of exemplary placental products of the present technology and two commercially available products (results adjusted per 8 cm² after subtraction of the negative background). AM75 and AM 78 are placental products of the present technology (e.g. cryopreserved) and CM75 and CM78 are cryopreserved chorionic membrane products.

TABLE 14
Therapeutic factors present in Supernatants of Exemplary Placental Tissues
and Commercially Available Products (pg/ml/8 cm²).

| Factor | Units | Apligraf | Dermagraft | AM75 | CM75 | AM78 | CM78 |
|---|---|---|---|---|---|---|---|
| hMMP1 | pg/ml/8 cm² | 1964945.37 | 14818.20 | 2821.85 | 3531.81 | 117326.89 | 95.46 |
| hMMP7 | pg/ml/8 cm² | 911.54 | 0.00 | 0.00 | 0.00 | 3.96 | 0.00 |
| hMMP10 | pg/ml/8 cm² | 1836.44 | 159361.33 | 993.34 | 465.47 | 1091.97 | 91.97 |
| hMMP13 | pg/ml/8 cm² | 21.61 | 0.00 | 0.00 | 0.00 | 0.71 | 0.00 |
| hMMP3 | pg/ml/8 cm² | 208281.70 | 180721.52 | 170.26 | 161.52 | 8325.17 | 0.00 |
| hMMP9 | pg/ml/8 cm² | 8872.28 | 19321.39 | 214.78 | 1455.11 | 630.56 | 57.59 |
| hMMP2 | pg/ml/8 cm² | 153341.77 | 19712.21 | 287.14 | 37.93 | 3823.38 | 24.44 |
| hMMP8 | pg/ml/8 cm² | 36.92 | 12.19 | 0.00 | 0.00 | 0.00 | 0.00 |
| hTIMP1 | pg/ml/8 cm² | 2487.18 | 10909.84 | 569.23 | 883.05 | 28743.48 | 97.94 |
| hTIMP2 | pg/ml/8 cm² | 7285.53 | 1796.56 | 89.29 | 13.72 | 424.06 | 4.83 |
| MMP/TIMP | | 239.26 | 19.72 | 6.81 | 6.26 | 4.50 | 2.62 |

Both MMPs and TIMPs are among the factors that are important for wound healing. However, expression of these proteins must be highly regulated and coordinated. Excess of MMPs versus TIMPs is a marker of poor chronic wound healing. We investigated expression of MMPs and TIMPs and its ratio in amniotic membrane and chorionic membrane and compared it to the expression profile in Apligraf and Dermagraft.

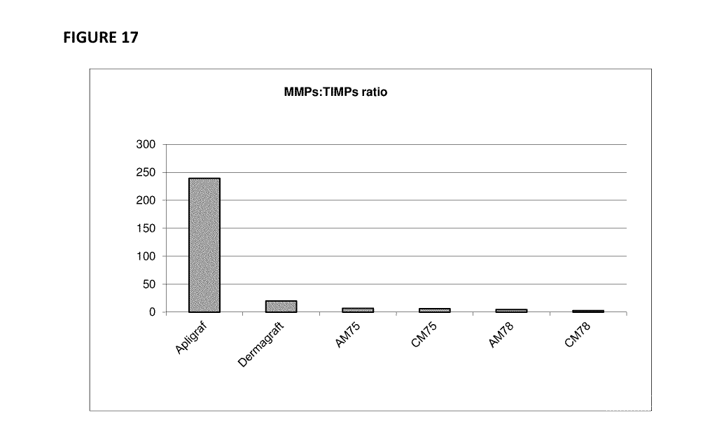

Results in Table 14 and FIG. 17 showed that all membranes express MMPs and TIMPs; however, the ratio in the thawed placental products and chorionic membranes is significantly lower. Therefore, these membranes will be more beneficial for wound healing (FIG. 17).

Accumulated data indicate that the MMP to TIMP ratio is higher in cases of non-healing wounds. For example, the ratio between MMP-9 and TIMP1 is approximately 7-10 to one for good healing and 18-20 or higher for poor healing. Analysis of the ratio between MMPs and TIMPs secreted by placental tissues, Apligraf, and Dermagraft showed that the amniotic and chorionic membrane products contain MMPs and TIMPs at an approximate ratio of 7, which is favorable for wound healing. In contrast, Dermagraft had a ratio >20, and Apligraf had a ratio >200.

These data are consistent with certain embodiments of the present technology that provide a placental product comprising an amniotic membrane containing MMP-9 and TIMP1 at a ratio of about 7-10 to one

### Example 17

### Establishment of EGF as a Marker for Amniotic Tissue Potency

EGF is among the factors that are important for wound healing (Schultz et al., 1991, Komarcevic, 2000, and Hong et al., 2006). The absence or decreased amount of EGF is one characteristic of chronic wounds (Harding et al., 2002). Evaluation of proteins derived from amniotic membrane samples prepared according to the developed manufacturing process disclosed by the present application reveal that EGF is one of the major factors secreted in higher quantities by these tissues. The importance of EGF for wound healing together with high levels of EGF detected in the presently disclosed amniotic membranes support selection of EGF as a potency marker for evaluation of membrane products manufactured for clinical use pursuant to the present disclosure. A commercially available ELISA kit from R&D Systems was selected for evaluation of its suitability to measure EGF secreted by amniotic membranes. ELISA qualification meets the standards established by the FDA and ICH guidances for bioanalytical assay validation (Validation of Analytical Procedures: Text and Methodology Q2(R1), 1994; ICH Harmonized Tripartite Guideline and Guidance for Industry Bioanalytical Method Validation, 2001). Amniotic membranes evaluated for expression of EGF by this method confirmed protein array data and further demonstrated that EGF was a unique factor expressed at clinically significant levels in these tissues.

### Example 17.1

### Amniotic Tissue Expression of EGF

Protein array analysis provided initial evidence that EGF was uniquely expressed in amniotic membranes but not in chorionic membranes (Table 15). The levels of EGF measured in amniotic membranes were of clinical significance.

TABLE 15
Protein array data showing range of expression of
EGF in amniotic and chorionic membranes from
multiple donors.

| | Amnion | Chorion |
|---|---|---|
| | (pg/ml) | (pg/ml) |
| EGF | 127.3-361.4 | 0-0.8 |

These data are consistent with certain embodiments of the present technology that provide a placental product comprising an amniotic membrane containing EGF, optionally in substantial amounts

### Homogenate of Placental Products

Placental products were thawed until no ice crystals were present. Membranes were then removed from bags and cut into 4 cm×2 cm pieces while still adhered to nitrocellulose. Each piece of tissue was then removed from the nitrocellulose and washed twice with PBS. Each tissue was then snap frozen in a homogenization tube using liquid nitrogen. Subsequently, one pre-cooled 5 mm steel bead was added to each tube; samples were then homogenized using a Qiagen Tissue Lyser according to the manufacturer's recommendations in 500 μL homogenization media. Tissue homogenates were stored at −80° C.±5° C. until analyzed by ELISA for EGF expression.

### EGF Expression in Amniotic Membranes

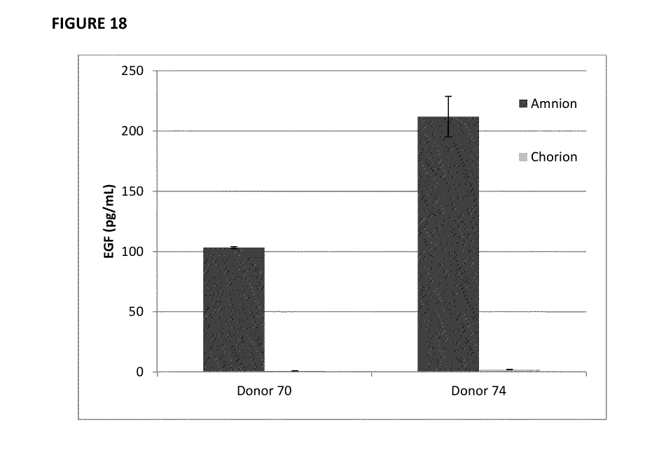

Measurement of EGF in amniotic preparations has proven to be both reliable and reproducible. Measurement of EGF in multiple donors showed that this method of quantification was a valuable means of evaluating potency in tissue prepared pursuant to the present disclosure for use in a clinical setting. FIG. 18 shows representative expression of EGF in a thawed placental product and chorionic membrane prepared and analyzed by the methods described above. Results have been reproduced in multiple tissue preparations.

These data are consistent with certain embodiments of the present technology that provide a placental product comprising an amniotic membrane containing EGF.

### Functional Studies:

### Example 18

### Immunomodulatory Effects of Placental Membranes

Chronic wounds fail to progress through the normal stages of healing, often stalling in the inflammatory stage (Maxson, Lopez et al. 2012). The characteristics of the chronic wounds environment include 1) high levels of proinflammatory cytokines such as TNF-α and IL-1α, 2) low levels of anti-inflammatory cytokines, 3) high levels of proteases and low levels of their inhibitors, as well as 4) high levels of oxidants and low levels of antioxidant to counter balance. Therefore inflammation control at the site of injury is the key to restart/progress the healing process. The effect of placental membranes on anti-inflammatory activity was investigated for wound healing.

### Example 18.1

### Tumor Necrosis Factor Alpha (TNF-α) Stimulation

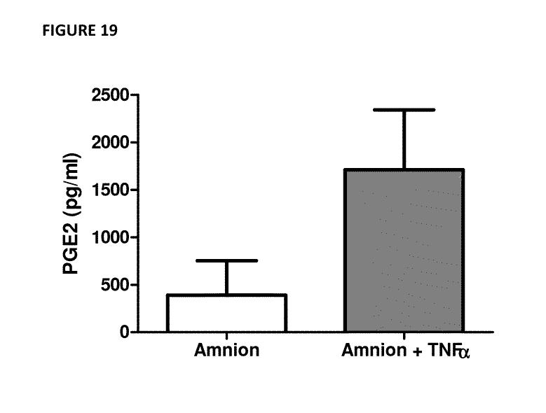

Tissue samples were cultured with and without 1 ng/ml of TNF-α (proinflammatory factor) for 18 hours. Supernatants were collected and the concentration of prostaglandin E2 (PGE2) was then measured using PGE2 monoclonal enzyme immunoassay assay (EIA) kit (Cayman). FIG. 19 showed that our placental membrane products (amniotic membrane product) produce high levels of anti-inflammatory cytokine PGE2 when exposed to TNF-α.

### Example 18.2

### Peripheral Blood Mononuclear Cells (PBMC) Assay

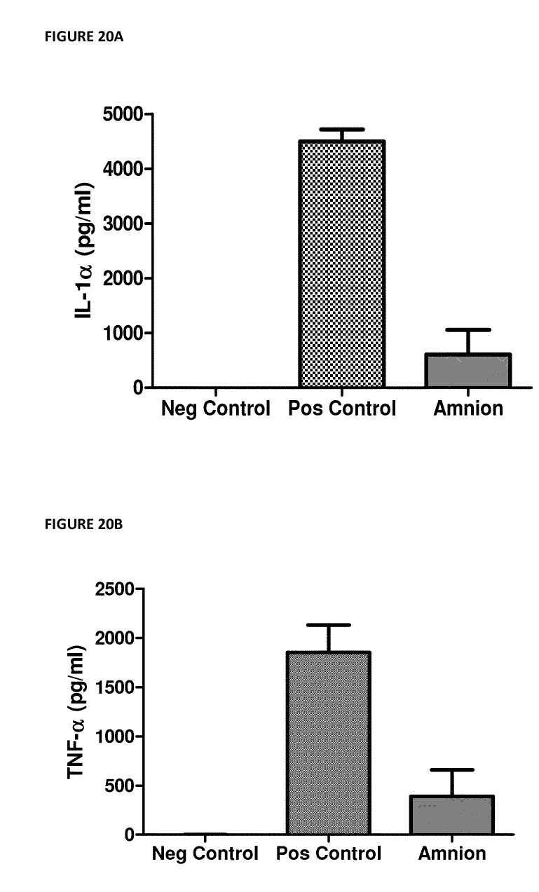

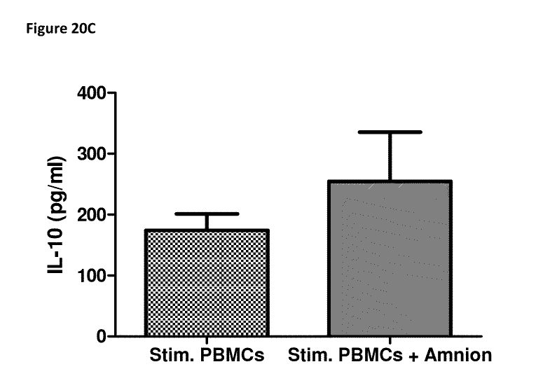

PBMC were obtained from SeraCare Life sciences. All experiments were performed in duplicates in 24-well plates with 10⁶ mononuclear cells in 1 mL assay medium per well. To examine the inhibitory effect of pro-inflammatory cytokines, T cell mitogens-anti-CD3 monoclonal antibodies (CD3) and anti-CD28 monoclonal antibodies (CD28) were added at 1 μg per mL to activate immune cells. Tissue samples were then incubated with activated PBMCs for 48 hours at 37° C. and 5% CO₂ in a humidified atmosphere. TNF-α, and IL-1α production were measured in supernatants using ELISA Duoset kits (R&D systems). To examine the release of anti-inflammatory factor IL-10, we pre-stimulated PBMCs with 100 ng/ml LPS for 4 hours, and then incubated tissue samples (pre-stimulated overnight with 10 ng/mL TNF-α) with the stimulated PBMCs for additional 24 hours. IL-10 was detected in the supernatant using IL-10 Duoset kit (R&D Systems). PBMCs without LPS stimulation was included as negative controls. The placental membrane product largely inhibited the release of the soluble proinflammatory cytokines (TNF-α, and IL-1α) and upregulated the release of anti-inflammatory IL-10 when co-cultured with activated immune cells (shown in FIG. 20).

### Example 18.3

### Regulation of Elevated Levels of Proteases by Amniotic Membranes

The prolonged inflammatory reaction in chronic wounds generates an intensified protease response, in particular with increased MMPs and neutrophil elastase activity. MMPs and elastase are involved in normal physiological and pathologic processes, such as degradation of basement membrane, remodeling of ECM, connective tissue turnover, angiogenesis, reproduction and wound repair. However, excessive MMPs and elastase destroy components of ECM and damage growth factors and their receptors that are essential for healing. In this study, we investigated whether placental membrane products mediate the inhibition of MMPs and elastase.

### Azocoll Assay for MMP Activity

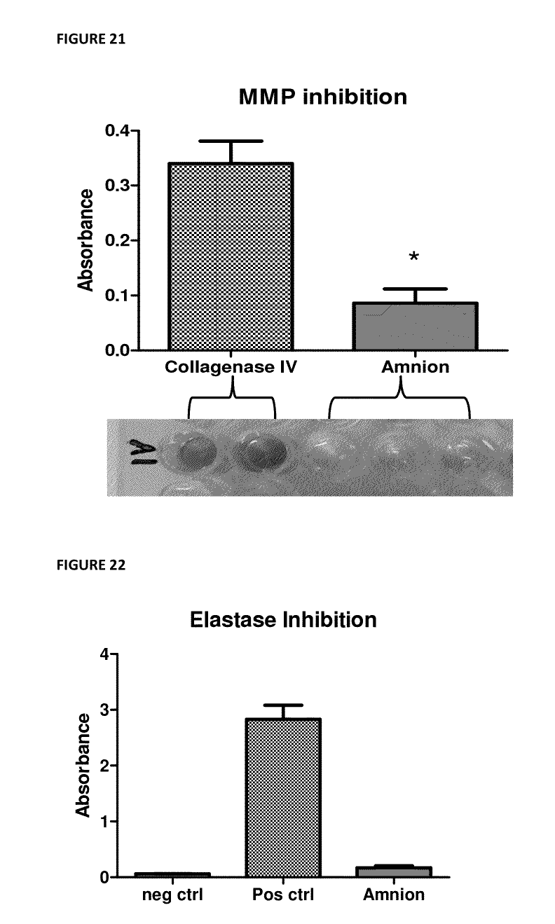

Azocoll is an insoluble, ground collagen to which a purple azodye is impregnated. Upon proteolysis, soluble azodye is released and can be detected by absorbance at 550 nm. Therefore, azocoll is often used as a chromogenic non-specific substrate to examine the protease activity in the environment. The assay was performed using a modified method developed by Jiang et al (Jiang, Tan et al. 2007). Azocoll was washed and suspended in 10 mM PBS, pH 7.4, at a final concentration of 1.5 mg/mL. Collagenase IV was used as the positive control because its two active forms, MMP-2 and MMP-9 (72 kD and 92 kD, respectively), have been shown to have elevated expression in wound fluid from chronic leg ulcer, which is correlated with poor healing (Trengove, Stacey et al. 1999). Amniotic membrane product was incubated with 0.1% (w/v) collagenase IV (Life Technologies) and azocoll suspension under gentle end-to-end rotation at 37° C. incubator for 5 hours. The reaction was stopped by centrifuging the samples at 10,000×g for 8 minutes. The absorbance of the supernatant solution was measured at 550 nm using an ELISA reader (spectramax) and two-tailed Student's T-test was performed to determine statistical significance (p<0.05) FIG. 21 demonstrates that cryopreserved amnion can significantly inhibit MMP activity.

### Neutrophil Elastase Assay

Amniotic membrane products were pre-conditioned with 100 ng purified human neutrophil elastase (Sigma) in DMEM with 1% FBS for 24 hours. Pre-conditioned tissue samples and 100 ng fresh neutrophil elastase were incubated for 4 hours at 37° C. in a final volume of 500 μl of 0.1M HEPES buffer, pH 7.4, containing 0.5M NaCl, 10% DMSO and 1 mM elastase substrate (Sigma). Substrate degradation was continuously monitored by measuring OD₄₀₅. FIG. 22 illustrates that cryopreserved amnion inhibited elastase activity by approximately 94%.

### Example 18.4

### Neutralization of ROS in the Wounds

Immune cells such as neutrophils and macrophages produce reactive oxygen species (ROS). ROS in low concentrations provide signaling and defense against microorganisms. High amounts of ROS, however, not only damage extracellular structure proteins, lipids and DNA, but also enhance the expression of MMPs, serine proteases and inflammatory cytokines, which would impair wound healing. We first investigated the total antioxidant capacity of placental membrane products using an antioxidant capacity kit. Moreover, whether placental membrane products can protect dermal fibroblasts from oxidant-induced apoptosis was examined.

### Antioxidant Assay

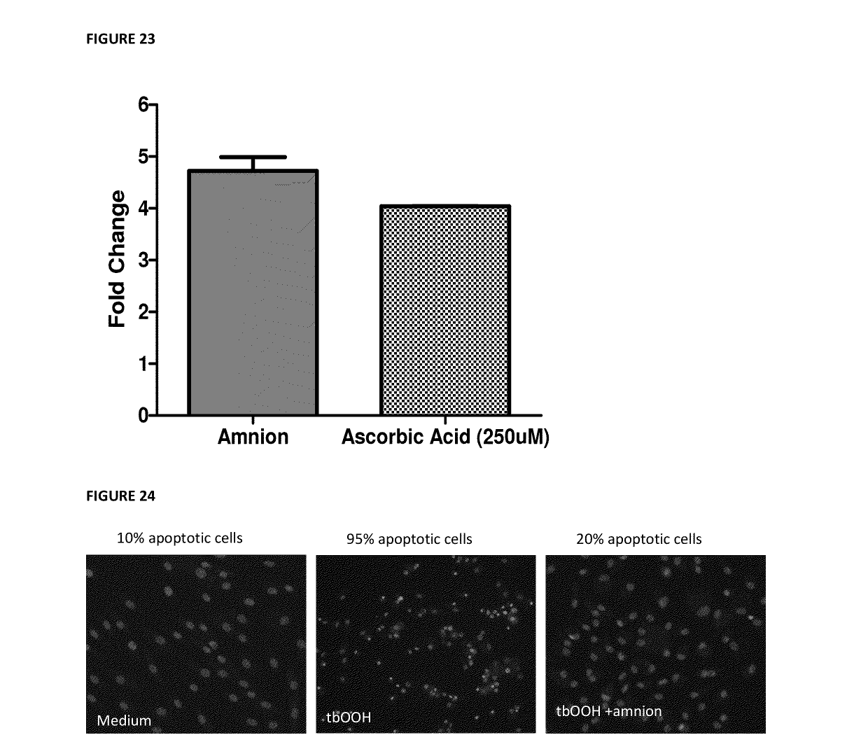

The antioxidant activities of conditioned medium from amniotic membrane products were measured using an antioxidant assay kit (Sigma CS0790) according to the manufacturer's instructions. The method is based on the release of a radical cation ABTS⁺ from ABTS, producing a green color signal when exposed to oxidative conditions. Inhibition of the color intensity produced can be related to the antioxidant capacity of a sample. FIG. 23 demonstrates that amniotic membrane products possess antioxidant capacity as potent as 250 μM ascorbic acid.

### Cell Survival Assay

Cell survival assay was tested using a modified method reported by Kim et al (Kim, Park et al. 2008). Normal human dermal fibroblasts (NHDFs) (Lonza) were plated at a density of 5×10⁴ cells/well in 24-well plates and starved for 24 hours in DMEM supplemented with 0.1% FBS. Subsequently, the organic hydroperoxide, tert-butyl hydroperoxide (tbOOH), was used as an oxidant to induce oxidative injury to NHDFs for 3 hours. Replace tbOOH containing medium with normal culture medium and incubate amniotic membrane products with HDFs to start rescuing process. The positive control group was replaced with 250 μM ascorbic acid. After 4 hours, cell apoptosis was determined by incubating NHDFs with 10 μg/ml Hoechst for 30 min at 37° C. and scoring the percentage of cells having intensely condensed chromatin and/or fragmented nuclei by fluorescence microscopy using ImageJ (NIH). FIG. 24 demonstrates that amniotic membrane products are able to rescue those early-stage apoptotic HDFs by 80%.

### Example 19

### Placental Membrane Products Enhance Angiogenesis

Neovascularization is a crucial step in the wound healing process. The formation of new blood vessels is necessary to provide the fibroblasts with sufficient nutrient supply for the production of a provisional granulation matrix and the survival of keratinyocytes.

### Tube Formation

To examine the angiogenic potential of amniotic membrane product, HUVECs (Lonza) were seeded with conditioned medium derived from amniotic membrane product, at a concentration of 10⁴ cells/well on Matrigel (BD)-coated culture wells. Conditioned medium was obtained by culture amniotic membrane products in endothelial cell growth medium EBM (Lonza) supplemented with 2% FBS for three days. EBM supplemented with all necessary cocktail of growth factors were used positive controls. After 8 hours of incubation, fields from each sample were randomly photographed by inverted microscopy, and the number of closed tubes were counted and plotted by ImageJ (NIH).

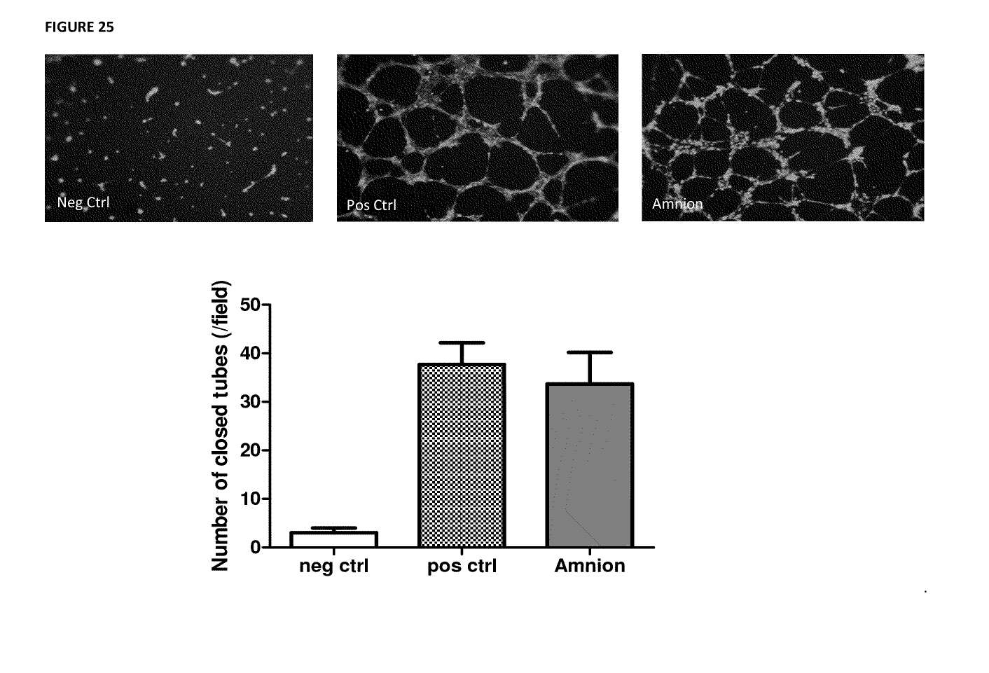

FIG. 25 demonstrates that conditioned medium from amniotic membrane products enhances tube formation, equivalent to positive control.

### Example 20

### Placental Tissues Enhance Cell Migration and Wound Healing

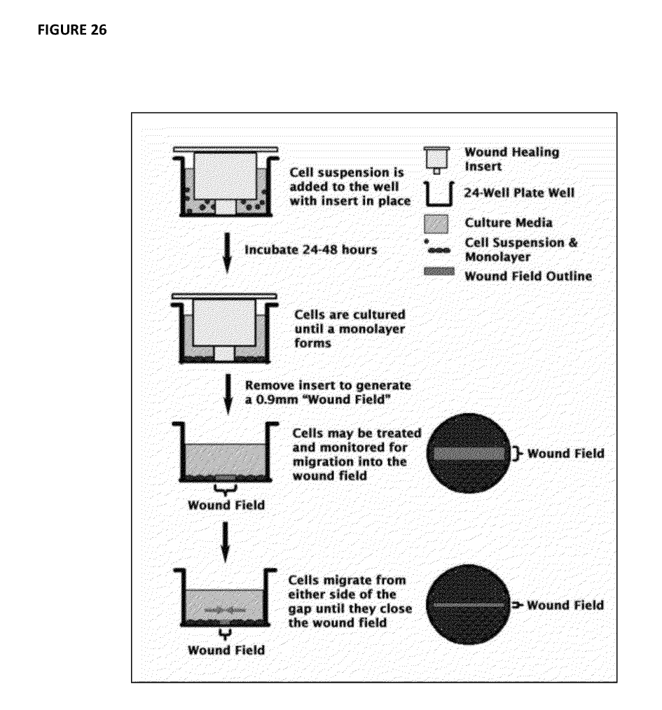

The process of wound healing is highly complex and involves a series of structured events controlled by growth factors (Goldman, 2004). These events include increased vascularization, infiltration by inflammatory immune cells, and increases in cell proliferation. The beginning stages of wound healing revolve around the ability of individual cells to polarize towards the wound and migrate into the wounded area In order to close the wound area and rebuild the surrounding tissue. Three types of cells are mainly involved in the cell migration process. They are vascular endothelial cells, fibroblasts and keratinocytes. Vascular endothelial cells migrate to the area of the wound and form new blood vessels, which provides the necessary oxygen and nutrients for proper wound healing. Fibroblasts need to migrate into the wound site to form granulation tissue, in order to reconstitute the various connective tissue components. Re-epithelialization (wound closure) requires the directional migration of keratinocytes. Therefore, we sought to determine if factors secreted from amniotic and chorionic membranes produced at Osiris promote these three types of cell migration and wound field closure. To accomplish this, we utilized a commercially available wound healing assay as seen in FIG. 26 (Cell Biolabs) and a transwell cell migration assay. Cell lines include human microvascular endothelial cells (HMVEC, Lonza Inc.), human umbilical endothelial cells (HUVEC, Lonza Inc.), human dermal fibroblasts (HDF, Lonza Inc.) and diseased human keratinocytes (D-HEK Lonza Inc.). Results indicate that cell migration of all three types of cells is enhanced by treatment with conditioned media from placental membranes.

### Example 20.1

### Placental Membrane Conditioned Media Supports Endothelial Cell Migration and Wound Field Closure

Cells are collected via trypsinization, pelleted, and counted before being resuspended in complete keratinocyte media at a density of 2×10⁵ cells/mL. 250 ul (5×10⁴ cells) of cell suspension is then pipetted into each side of a well containing a wound healing insert (Cytoselect 24 well Wound Healing Assay Plate, Cell Biolabs). The cells are grown for 24 hours in complete media. After 24 hours, the wound inserts are removed. At the same time, complete keratinocyte media is removed and replaced with experimental media. Complete keratinocyte media and basal keratinocyte media were used as positive and negative controls, respectively. To generate experimental media, placental membranes are incubated for three days in DMEM with 1% human serum albumin (HSA) in a tissue culture incubator. The resulting tissue and media are then placed in eppendorf tubes and spun at high speed in a microcentrifuge. The supernatants are collected and stored at −80 C until use. For migration and wound healing studies, conditioned media from placental membranes is diluted 1:20 in basal keratinocyte media before being added to experimental wells. After 18 hours, the media is removed and the cells are fixed for 20 min in 4% parafolmaldehyde and stained with crystal violet. The wound field in each well is then photographed. Wound healing in determined by the amount of wound field still visible at the end of the experiment when compared to control pictures taken before conditioned media is added to the wells.

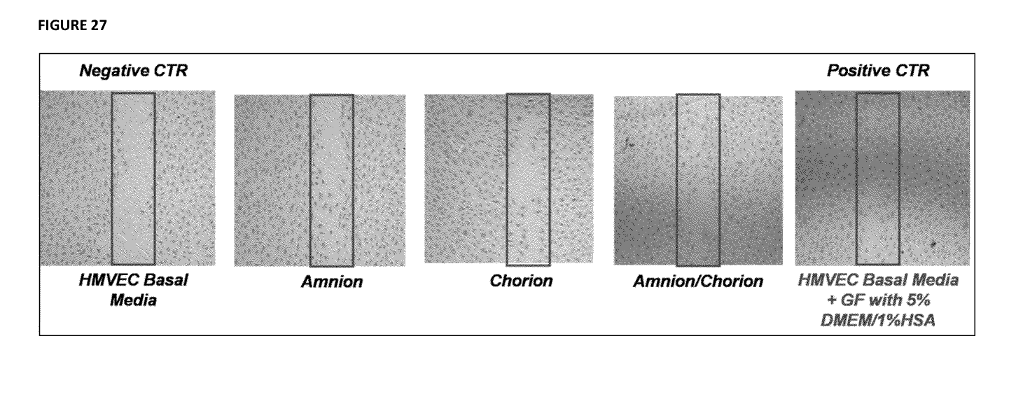

Conditioned media from amniotic and chorionic membranes was used to assess the potential for these membrane to promote endothelial cell migration and wound field closure. Conditioned media from placental amniotic, chorionic, and a combination of amniotic/chorionic membranes supported migration of endothelial cells into the experimental wound field (FIG. 27)

### Example 20.2

### Placental Membrane Conditioned Media Supports Endothelial Cells, Fibroblasts, and Keratinocytes Migration

### Transwell Cell Migration Assay

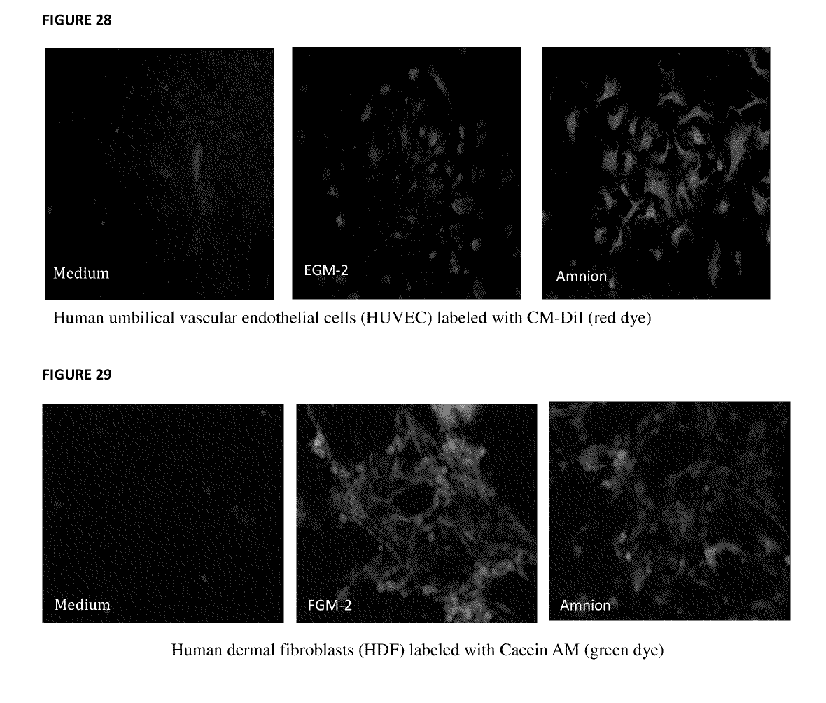

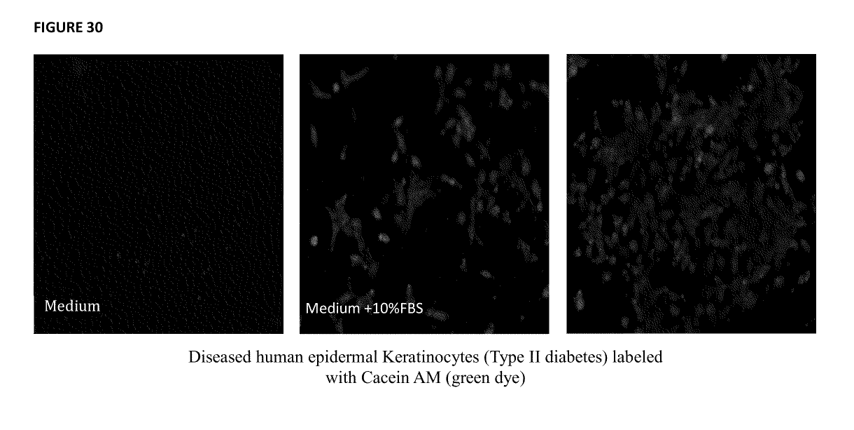

Migration assay was performed on human umbilical vein endothelial cells, human normal dermal fibroblasts, and human diseased keratinocytes (type II diabetes) (Lonza) using a FluoroBlok transwell system with 8 μm pores (BD). 100,000 cells were suspended in DMEM with 0.1% FBS and added to the upper chamber of the transwell to arrest mitosis. The next day, conditioned medium derived from tissue samples were added to lower chambers and incubated overnight. After incubation, cells that had migrated through the filter were fixed and stained for calcein (Molecular Probe). The field was visualized by a fluorescence inverted microscope and pictures were taken from four randomly chosen fields under 10× magnification. Our results demonstrated that amniotic products promote the migration of endothelial cells (FIGS. 27 and 28), fibroblasts (FIG. 29), and keratinocytes (FIG. 30).

### Clinical Studies

### Example 21

### Use of Placental Products for Treating Diabetic Foot Ulcers

Purpose:

Despite bioengineered skin substitutes that contain human fibroblasts or a combination of human fibroblasts and keratinocytes, published rates of chronic wound healing remain low, with approximately half of all wounds recalcitrant to even these newer therapies. Morbidity and mortality from diabetic foot ulceration are substantial as the 5 year mortality rate following a lower extremity amputation is between 39% and 68%. (Page J. J of Foot & Ankle Surgery 2002; 41(4):251-259; Isumi Y., et al. Diabetes Res and Clin Practice 2009; 83:126-131).

A cryopreserved placental membrane product, which provides necessary angiogenic and anti-inflammatory growth factors was introduced in an effort to improve outcomes of patients with chronic skin ulceration at-risk for amputation.

Objective:

Patients with chronic diabetic foot ulceration, unresponsive to available therapy and at-risk for amputation were considered for treatment. All wounds were aggressively debrided prior to application of cryopreserved placental membrane. Patients were evaluated regularly and application of a membrane product of the present technology was at the discretion of the treating physician. Offloading was encouraged in both patients.

### Introduction

According to the United States Food and Drug Administration (FDA), a chronic, cutaneous ulcer is defined as a wound that has failed to proceed through an orderly and timely series of events to produce a durable structural, functional and cosmetic closure (2). The most common chronic wounds include pressure ulcers and leg ulcers. The triad of peripheral neuropathy, deformity, and minor trauma has emerged as the most frequent causes of insult that lead to foot ulcerations. In terms of healing rates, an appropriate benchmark for a chronic wound is a decrease of 10% to 15% in size every week, or 50% decrease in size over a one-month period. A three-year retrospective cohort study performed by Ramsey et al. of 8,905 patients in a large health maintenance organization who have diabetes reported a 5.8% cumulative incidence of ulceration. At the time of diagnosis, 15% of these patients developed osteomyelitis and 16% required partial amputation of a lower limb.

Approximately 80% to 85% of lower extremity amputations are preceded by foot ulcerations. Morbidity and mortality from diabetic foot ulceration are substantial as the 5 year mortality rate following a lower extremity amputation is between 39% and 68%. (2). These mortality rates are higher than the five-year mortality rates for breast cancer, colon cancer, and prostate cancer.

Despite all of the advances in bioengineered tissue for the treatment of chronic diabetic ulcerations, there are an abundance of patients whose ulcerations are resistant to therapy, and result in a chronic wound. Because of healing rates that only approach 50% with these newer therapies, the use of stem cells in regenerative medicine has been of particular interest recently. The ultimate aim is to promote restoration of functional skin. A preliminary study was performed by Fiami et al. in which they isolated mesenchymal stem cells from umbilical cord blood and inoculated them onto a piece of de-epithelialized dermis. The results of this preliminary study showed that peripheral stem cells are capable of surviving and expressing neoangiogenesis. In addition to showing promise for tissue repair, mesenchymal stem cells exhibit low immunogenicity and can be transplanted universally without having to undergo compatibility testing between the donor and recipient.

In this study, clinical evidence of remarkable healing using a cryopreserved membrane product for the treatment of two chronic wounds that amputation was considered. The fundamentals of wound management are still the cornerstone of comprehensive wound care in any treatment protocol including adequate debridement, offloading, maintaining a moist environment, and adequate perfusion and infection control.

### Materials

A cryopreserved placental membrane was made as described herein, comprising an allograft derived from the amnion including a bilayer of native epithelial cells and a stromal layer consisting of neonatal fibroblasts, extracellular matrix (ECM) and mesenchymal stem cells (MSC).

### Limb Salvage: Case One

### History and Physical Examination

A 70 year old male presented to the emergency department with bulla formation on the dorsolateral aspect of his right foot between the fourth and fifth digits, edema and pain, and a small lesion lateral to the fifth digit. The patient reported a history of minor trauma to the area two weeks prior to presentation. The patient had a history of type II diabetes mellitus, hypertension, heart failure, chronic obstructive pulmonary disease, and chronic kidney disease treated with hemodialysis three times a week. The patient had a surgical history of an aorta-venous graft replacement. He denied any history of alcohol, tobacco or drug use. Physical exam revealed no active purulent drainage or malodor, and no tenderness on palpation. The vascular exam revealed non-palpable pulses in the dorsalis pedis and posterior tibial arteries. Doppler exam revealed a monophasic dorsalis pedis pulse with a biphasic posterior tibial artery pulse. The fifth digit had gangrenous changes and was cold on palpation. There were ischemic changes of the fourth digit. Radiographic evaluation revealed scattered air densities indicative of soft tissue gas in the fourth interspace as well as the tip of the fifth digit.

### Preoperative Management

The patient was started on intravenous antibiotics of vancomycin and piperacillin and tazobactam at appropriate renal dosing.

### Operative Management

The patient was taken to the operating room where an incision and drainage of the fourth interspace was performed, and a partial fifth ray amputation to the level of the metatarsal head was performed without complication. The wound was left open and packed with sterile gauze moistened with sterile normal saline, and covered with a sterile compressive dressing. Intraoperative findings revealed liquefactive necrosis of surrounding tissues with purulence and malodor. The patient underwent 2 subsequent surgical debridements, with the second resulting in further removal of the fourth and fifth metatarsal shafts. In a third surgery further debridement of necrotic soft tissue and amputation of the fourth digit was performed. On May 20, 2010 treatment with a cryopreserved placental membrane was initiated. Prior to the graft placement the patient had undergone successful recanalization of the popliteal artery and the peroneal artery without significant residual stenosis.

### Postoperative Course

The patient followed up with his podiatric surgeon within 2 days of being discharged from the hospital. Upon initial exam, there were no clinical signs of infection, and the proximal dorsal incision appeared coapted. The third digit was dusky and cool in appearance. Radiographs were taken which showed no evidence of soft tissue gas or acute osteomyelitis. A dry sterile dressing was applied. The patient received applications of the cryopreserved placental membrane at 6 additional visits in an outpatient office. Prior to each application the wound was evaluated for abscess, cellulitis, drainage, hematoma formation, and infection. At each visit, the wound decreased in size and appeared more granular in nature as compared to previous visits. At the time of the third application the wound had decreased in size 50%.

At 19 weeks the wound was considered closed, and the patient was instructed to remain weight bearing on the affected limb with the use of a surgical shoe only.

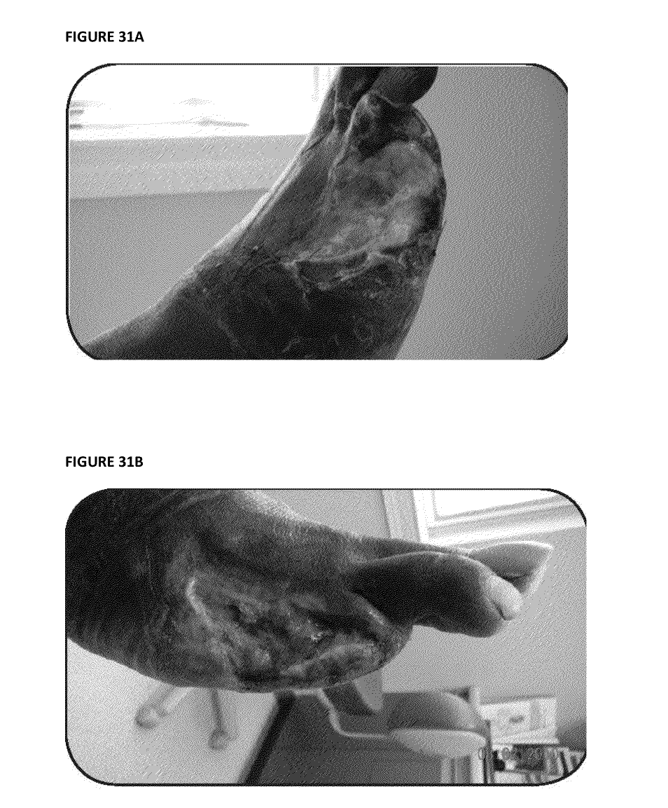

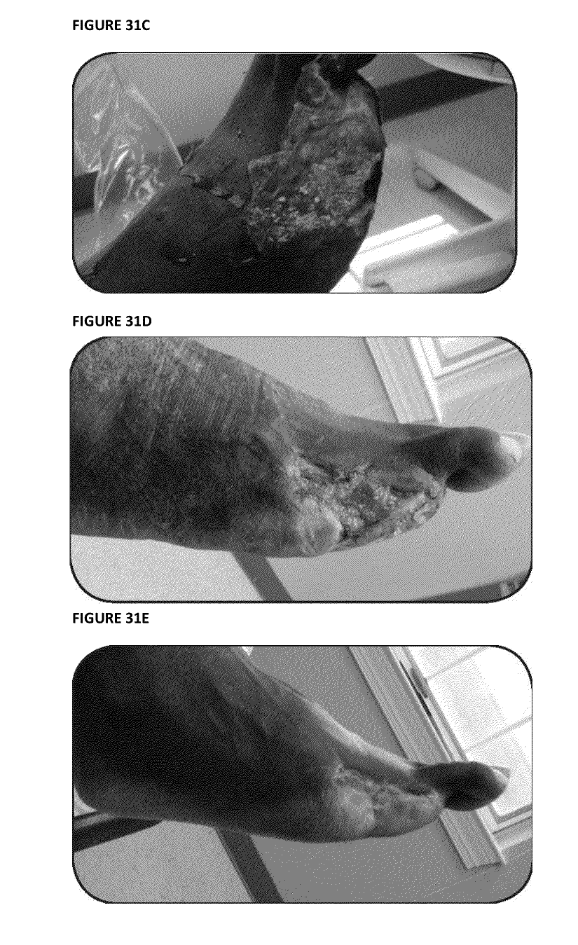

Photographs of the remarkable wound healing mediated by a placental product of the present technology as shown in FIG. 31. Panel A: First application of a cryopreserved placental membrane product; B: 8 weeks post first cryopreserved placental membrane an instant membrane product application; C: 10½ weeks post first cryopreserved placental membrane an instant membrane product application; D: 12 weeks post first cryopreserved placental membrane an instant membrane product application; E: 19 weeks post first cryopreserved placental membrane an instant membrane product application.

### Limb Salvage: Case Two

### History and Physical Examination

A 44-year old male presented to an outpatient office with a large ulceration on the plantar aspect of his left hallux, secondary to a previous trauma a few weeks prior to the visit. The patient had a history of diabetes mellitus for the past five years complicated by peripheral neuropathy, hypertension, dyslipidemia, and osteomyelitis. Past surgical history included abdominal aortic aneurysm repair and circumcision. On physical exam the ulceration measured 4.0 cm×2.0 cm×1.5 cm, probing to the distal phalanx with exposed tendon. There was no ascending cellulitis or lymphangitis, and no increased temperature gradient. Capillary fill time, hair growth, and tissue turgor were all normal. There were palpable pulses in the dorsalis pedis and the posterior tibial artery. Radiographic exam was negative for soft tissue gas. Magnetic resonance imaging revealed osteomyelitis in the distal aspect of the proximal phalanx and the distal phalanx of the great toe with a small soft tissue abscess in the region of the dorsal soft tissue adjacent to the distal phalanx.

### Preoperative Management

The patient was started on intravenous antibiotics. He was taken to the operating room for excisional debridement of all nonviable tissue and application of the instant membrane product.

### Operative Management

The ulceration was debrided to healthy tissue with utilization of both sharp dissection and VERSAJET™, leaving the head of the proximal phalanx exposed plantarly. The cryopreserved placental membrane product was then placed over the wound bed and exposed bone. The patient tolerated the procedure without complication. The patient was discharged from the hospital the day after surgery on a five week course of intravenous antibiotic therapy.

### Postoperative Course

The patient was instructed to remain strictly non-weight bearing to the affected limb, and returned for follow-up on post-operative day 6. The dressing was clean, dry and intact. There were no post-operative complications such as abscess, cellulitis, discomfort, or drainage and no clinical signs of infection. The patient received a total of 7 applications of cryopreserved placental membrane product over the course of the next 8 weeks. At each visit the wound was inspected for clinical signs of infection. Evaluation at each visit revealed marked development in granulation tissue to the wound base and significant decrease in size. Eight weeks after the initial application of the allograft tissue the wound was closed.

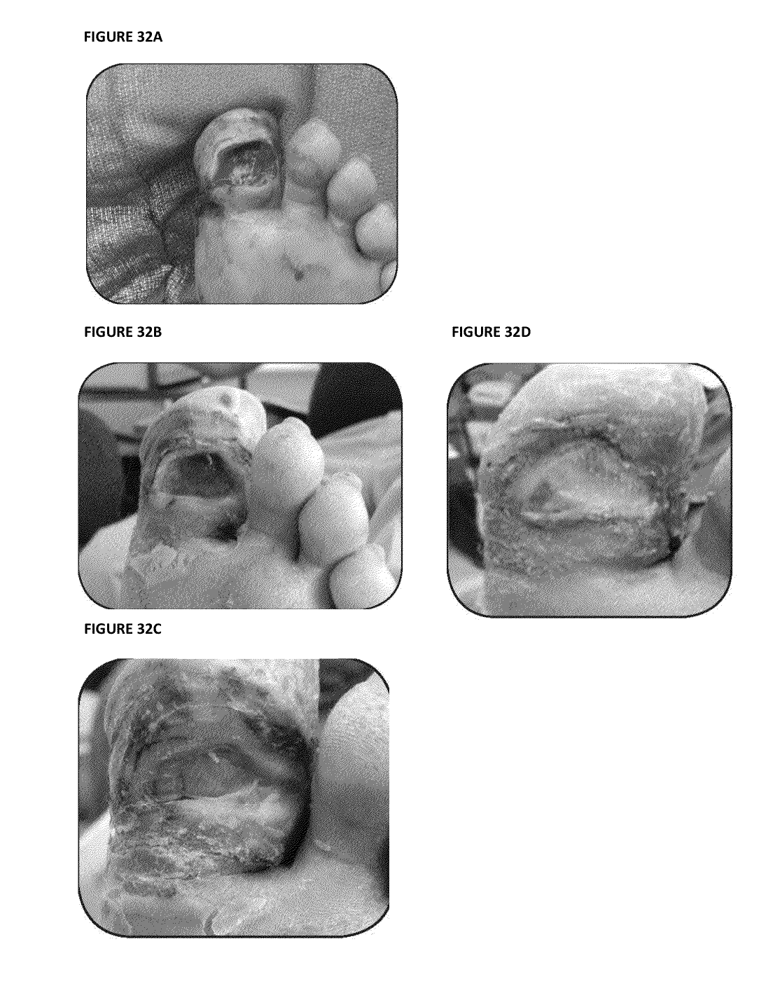

Photographs of the remarkable wound healing mediated by a placental product of the present technology as shown in FIG. 32. Panel A: Osteomyelitis, tendon exposed, probed to bone. First cryopreserved placental membrane graft was applied after surgical debridement; B: Status post 1 application of cryopreserved placental membrane graft, wound is granular in nature and no signs of infection; C: 3 weeks post-surgical intervention; 2 applications on the cryopreserved placental membrane graft, the wound is considerably smaller in circumference and depth; D: 6 weeks post-surgical intervention the wound is almost closed; At 8 weeks and 7 applications of the cryopreserved placental membrane graft, the wound is closed.

### Conclusion

Despite the tremendous progress in skin tissue engineering in the past few decades, current therapy has limited efficacy in the treatment of chronic diabetic ulceration. As shown in this case report of two patients, the use of advanced therapies containing stem cells may prove useful to ultimately heal these patients in lieu of amputation, reduce mortality rates, and at the same time be a cost effective alternative to standard treatments currently on the market. Both patients highlighted in this case report received 7 applications of a membrane product of the present technology. Complete healing occurred in both patients. There were no reported complications associated with treatment; the instant membrane product was safe and effective in an initial evaluation of two patients with diabetic foot ulceration at-risk for amputation. These results indicate that patients with recalcitrant, chronic wounds should be considered for this novel therapy.

### Example 22

### Evaluation of the Efficacy and Safety of Placental Membrane for the Treatment of Chronic Diabetic Foot Ulcers in Prospective Clinical Trial Study

Diabetic foot ulcers are a world-wide epidemic leading to significant morbidity and rising healthcare costs. This study evaluated the efficacy and safety of a cryopreserved placental membrane (N=50) compared to standard wound care (N=47) to heal chronic diabetic foot ulcers. This example is part of a prospective, multicenter, randomized, single-blinded clinical trial to evaluate the safety and efficacy of cryopreserved placental membrane products as described herein (e.g., Grafix®, Osiris Therapeutics, Columbia, Md.) that are derived from amniotic membranes for the treatment of chronic diabetic foot ulcers. The study conformed to the ethical guidelines of the 1975 Declaration of Helsinki and was registered with ClinicalTrials.gov (NCT01596920). Patients reviewed and signed a standard IRB approved consent form prior to enrollment. Patients were enrolled from May 2012 through April of 2013.

Key inclusion criteria included confirmed Type I or Type II diabetes, patient age between 18 years and 80 years, index wound present between 4 weeks and 52 weeks, wound located below the malleoli on plantar or dorsal surface of the foot, and ulcer between 1 cm² and 15 cm². Main exclusion criteria included hemoglobin A1c above 12%, evidence of active infection including osteomyelitis or cellulitis, inadequate circulation to the affected foot defined by an ankle brachial index <0.70 or >1.30, or toe brachial index ≦0.50 or Doppler study with inadequate arterial pulsation, exposed muscle, tendon, bone or joint capsule, and reduction of wound area by ≧30% during the screening period.

Following a one (1) week screening period, patients were randomized to the cryopreserved placental product or control treatment arm in a 1:1 ratio. Patients randomized to placental product treatment received an application of placental product once a week (±3 days) for up to 84 days (Blinded Treatment Phase). Patients in the control group received standard wound therapy once a week (±3 days) for up to 84 days. All wounds were appropriately cleaned and surgically debrided to remove all nonviable soft tissue from the wound by scalpel, tissue nippers and/or curettes at each weekly visit. For patients randomized to the placental product group, the cryopreserved placental membrane was placed to come in full contact with the wound and edges. Wounds in both groups received standard wound care which included surgical debridement, off-loading, and non-adherent dressings. All patients received a non-adherent dressing: ADAPTIC® (Systagenix, Gatwick, UK) and either saline moistened gauze or ALLEVYN® (Smith and Nephew, London, UK) for moderately draining wounds. An outer dressing was then applied. Patients were provided walking boots for wounds on the sole of the foot or a post-operative shoe if the wound was on the dorsum of the foot or at the ankle. Custorn off-loading boots could be prescribed at the discretion of the site investigator. In addition, the off-loading device used could be changed as needed to accommodate changes in wound size or position.

Patients were evaluated weekly at the clinical site. Patients who achieved complete wound closure then continued to be evaluated during the Follow-up Phase, twice during the first month and then monthly for two additional visits. Control patients whose wounds were not closed by the end of the Blinded Treatment Phase were able to receive placental product in the Open-Label Treatment Phase, in which the placental product (GRAFIX®) was applied weekly for up to 84 days. Outcome and safety assessments occurred at each visit during the Blinded Treatment Phase, Follow-up Visits, as well as during the Open-Label Treatment Phase.

The primary endpoint of the study was evaluation of complete wound closure of the index wound. Complete wound closure was defined as 100% re-epithelialization with no wound drainage as determined by the site investigator. Confirmation of wound closure was made at an initial follow-up visit 2 weeks later. Wound closure was independently confirmed via a central wound core laboratory with two blinded wound care experts who reviewed all wounds via digitized acetate tracing and photography. The secondary objectives included the time to initial wound closure among patients who received placental product versus those who received control as measured by Kaplan-Meier analysis. The proportion of patients that achieved 50% or greater reduction in wound size by 28 days, the number of applications needed for closure, and wound recurrence after initial wound healing was also determined. In the Open-Label Treatment Phase, wound closure with placental product for patients who were in the control group in the Blinded Treatment Phase was assessed. Safety assessments included the number, type, and severity of adverse events as outlined in National Cancer Institute's (NCI) common terminology criteria for adverse events (CTCAE) version 3.

Sample Size and Statistical Analysis:

The study sample size was based on an assumed closure rate of 30% in the control arm and 50% in the placental product group with a 30% drop out rate. Under these assumptions, 94 completed patients in each treatment arm were required to meet the 2-sided Type 1 error rate of 0.05 with 80% power. A pre-specified interim analysis was planned at 50% enrollment. The interim analysis utilized a one-sided superiority design based on an Emerson-Fleming symmetric group sequential design using an O'Brien-Fleming boundary shape (Emerson and Fleming, 1989). The analysis was performed by an unblinded statistician and reported to the blinded review committee. Following the interim analysis, the blinded review committee recommended to terminate study enrollment due to overwhelming superiority of the placental product arm versus the control arm.

The statistical analyses were performed using SAS version 9.2 on an intent-to-treat basis. Baseline demographic and clinical variables were summarized for each treatment arm of the study. Descriptive summaries of the distribution of continuous variables included the mean, standard deviation, median, and subject counts; categorical variables were summarized in terms of frequencies and percentages. Treatment group summaries were constructed across all study sites. Statistical comparisons between treatment groups were performed using Chi-square testing for categorical variables and analysis of variance (ANOVA) techniques for continuous measures. A Cox proportional hazard regression analysis was performed on time-to-event (wound closure) data.

### Results

Patient demographics and baseline characteristics are presented in Table 16. During screening, 139 patients were evaluated. There were 42 patients who failed screening, of which 6 were disqualified after the one week run in period because there was a 30% or greater wound area reduction. Ninety-seven patients were subsequently randomized; 50 received placental product, and 47 received standard wound therapy. There were no significant differences in baseline characteristics among the two treatment groups. The planned interim analysis showed overwhelming efficacy among patients that received placental product for the primary and secondary endpoints when compared to the control group (Table 17). Following the interim analysis, the blinded review committee recommended to terminate study enrollment due to overwhelming superiority.

### Efficacy Evaluation

Blinded Treatment Phase:

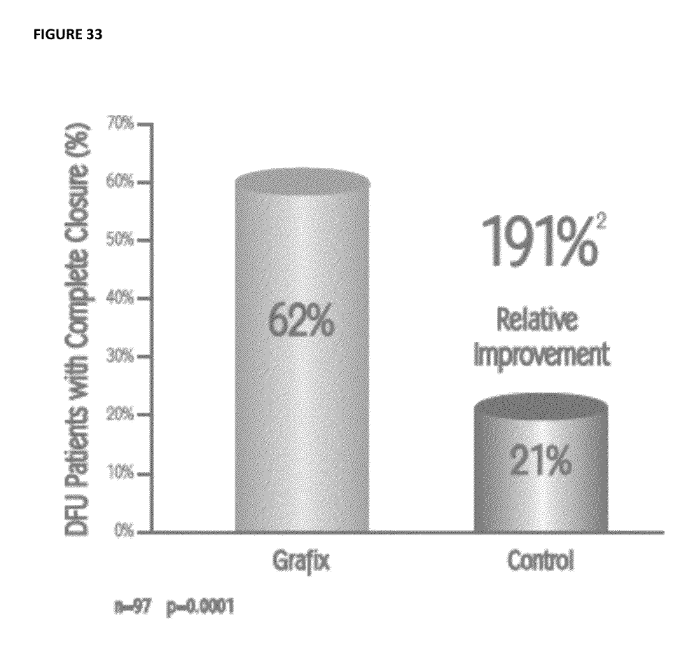

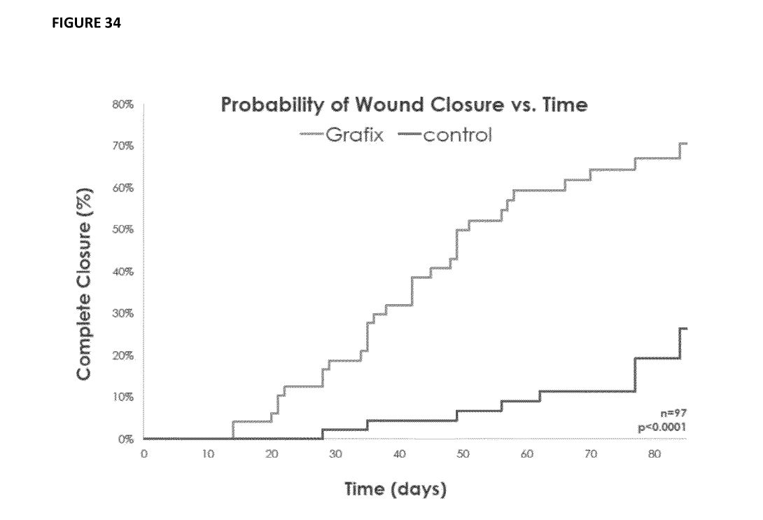

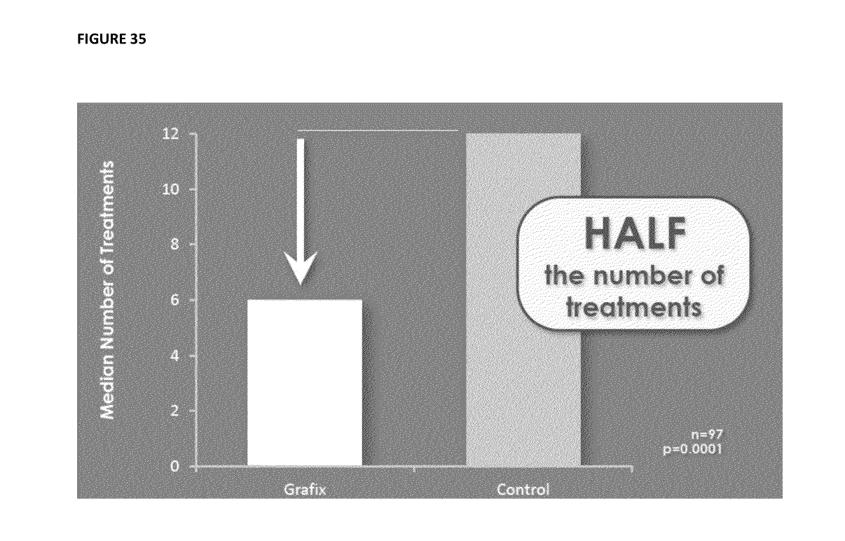

The proportion of patients that achieved complete wound closure was significantly higher in patients that received placental product (31 of 50, 62.0%) compared to controls (10 of 47, 21.0%, p=0.0001) as depicted in FIG. 33. The odds ratio for complete healing for a placental product patient compared to control was 6.037 (95% CI 2.449-14.882). The placental product group had significantly faster median time to complete wound closure compared to controls (42 vs. 69.5 days, p=0.019) among wounds in both groups that closed as depicted in FIG. 34. The Kaplan-Meier analysis illustrated a statistically greater probability of complete wound healing during the 12 week evaluation period for placental product (FIG. 34). The probability of closure for the placental product group was 67.1% compared to 27.1% for the standard care group (Log-Rank, p<0.0001). Placental product patients also required fewer study visits (i.e. applications) to achieve closure compared to patients in the control arm (6 vs. 12, p<0.001) as depicted in FIG. 35. Comparison of patients with at least a 50% reduction in wound size by Day 28, showed that 31 of 50 patients (62.0%) in the placental product group achieved this reduction versus 19 of 47 (40.4%) in the control group (p=0.035). There were 8 (16%) patients that withdrew from the study prior to completion in the placental product group versus 11 (23.4%) patients that withdrew from the control group.

Wound recurrence of DFUs closed during the initial 12 week study period was assessed. Follow-up at 2 weeks, 4 weeks and the every 4 weeks for a total of 12 weeks showed that ulcers remained closed in 82.1% of patients (23 of 28 patients) in the placental product group versus 70.0% (7 of 10 patients) in the control group (p=0.42).

Open-Label Phase:

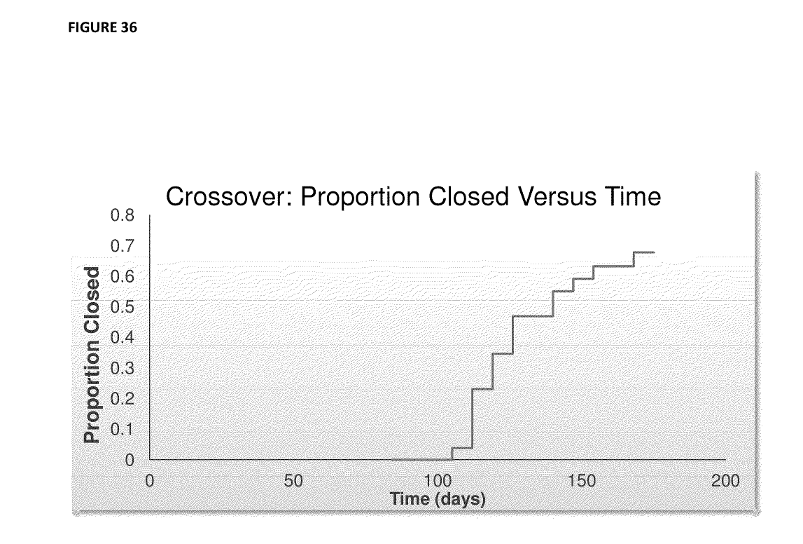

Patients in the control arm that failed to heal during the initial 12 week treatment period could cross-over to receive up to 12 weeks of placental product therapy (n=26). After receiving treatment with placental product, the probability of closure was 67.8% with a median time to closure for these patients of 42 days. (FIG. 36)

Regression Analysis:

Cox proportional hazard regression analysis was performed with treatment group, duration of ulcer, baseline ulcer area, glucose control (glycated hemoglobin), ulcer location, and BMI as covariates. Following adjustment for these variables, placental product was found to have a significant effect on time to closure (p<0.0001). The hazard ratio was 4.77 (95% CI 2.279, 9.971), indicating superior odds of closure with placental product relative to standard wound therapy.

Safety Evaluation:

Overall, fewer placental product patients experienced at least one adverse event compared to control patients (44.0% vs. 66.0%, p=0.031). Among patients randomized to placental product, there were significantly fewer patients with wound related infections (18.0% vs. 36.2%, p=0.044), fewer serious adverse events related to wound infections (8% vs. 21.3%, p=0.084) and fewer hospitalizations related to infections in the placental product group than control (6% vs. 15%, p=0.15). (Table 18).

### Discussion

The results of this study demonstrate that weekly application of a placental product as disclosed herein effectively improved healing rates of diabetic foot ulcers and reduced diabetic foot complications when compared to standard wound therapy. In this study all primary and secondary endpoints showed clinical benefit of the placental product, in the only multi-center DFU trial to date to meet statistically significant pre-specified interim analyses. This is also the first report of a multi-center randomized controlled trial (RCT) to investigate the use of human amniotic membrane for the treatment of diabetic foot ulcers. Additionally, to the authors' knowledge, this is the first large, multicenter RCT for advanced skin substitutes in which the primary endpoint, 100% re-epithelialization, was confirmed by 3^{rd} party blinded wound care experts, further removing potential bias and increasing reliability of the endpoint results. The placental product group had a significantly higher complete closure rate (191% relative improvement) as depicted in FIG. 33.

In this study surgical debridement was done for every patient at every study visit. Other studies have reported that a minority of DFUs received surgical debridement in phase 3 DFU trials (Steed, D. L., et al., Effect of extensive debridement and treatment on the healing of diabetic foot ulcers. Diabetic Ulcer Study Group. J Am Coll Surg, 1996. 183(1): p. 61-4; Saap, L. J. and V. Falanga, Debridement performance index and its correlation with complete closure of diabetic foot ulcers. Wound Repair Regen, 2002. 10(6): p. 354-9), and that the frequency of wound debridement was associated with differences in wound healing. (See, Steed et al., Supra; Table 19).

TABLE 16
Patient demographics and baseline characteristics

| | | | Placental | | | | |
|---|---|---|---|---|---|---|---|
| | Product | | Control | | | | |
| | (n = 50) | | (n = 47) | | | | |
| Mean age, in years (SD) | 55.5 | (11.5) | 55.1 | (12.0) | 0.849 | −5.20 | 4.30 |
| Age ≧65 years (N, %) | 11 | (22%) | 13 | (27.7%) | 0.521 | 0.292 | 1.861 |
| Male (N, %) | 33 | (66.0%) | 35 | (74.5%) | 0.365 | 0.276 | 1.603 |
| Mean years DM (SD) | 15.4 | (11.1) | 14.0 | (11.0) | 0.549 | −5.80 | 3.10 |
| Mean BMI (SD) | 33.5 | (7.7) | 32.2 | (7.9) | 0.419 | −4.40 | 1.90 |
| BMI ≧30 (N, %) | 36 | (72%) | 25 | (53.2%) | 0.057 | 0.975 | 5.253 |
| Race (N, %) | | | | | | | |
| White or Caucasian | 35 | (70%) | 32 | (68.1%) | 0.581 | −1.847 | 2.073 |
| Black or African | 13 | (26%) | 12 | (25.5%) | 0.521 | −1.866 | 2.054 |
| American | | | | | | | |
| American Indian or | 1 | (2%) | 1 | (2.1%) | 0.482 | −1.932 | 1.988 |
| Alaska Native | | | | | | | |
| Other | 1 | (2%) | 2 | (4.3%) | 0.263 | −1.947 | 1.973 |
| Mean wound size at | 3.41 | (3.23) | 3.93 | (3.22) | 0.433 | −0.80 | 1.80 |
| Baseline (cm², SD) | | | | | | | |
| Wound duration (days, SD) | 115.0 | (72.6) | 122.9 | (83.9) | 0.621 | −23.7 | 39.5 |
| Mean Glycated | 8.0 | (1.6) | 7.8 | (1.5) | 0.511 | −0.90 | 0.4 |
| hemoglobin (SD) | | | | | | | |
| Glycated hemoglobin | 14 | (28%) | 13 | (27.7%) | 0.970 | 0.418 | 2.473 |
| >9% (N, %) | | | | | | | |
| Mean Albumin g/dL (SD) | 4.0 | (0.4) | 4.0 | (0.3) | 0.418 | −0.20 | 0.10 |
| Albumin >3.5 g/dL (N, %) | 44 | (88%) | 42 | (89.4%) | 0.263 | −1.947 | 1.973 |
| Ankle Bachial Index | | | | | | | |
| (ABI) | | | | | | | |
| ABI 0.07-0.90 (N, %) | 10 | (21.7%) | 10 | (22.2%) | 0.44 | −1.89 | 2.03 |
| ABI >0.90 | 36 | (78.3%) | 35 | (77.8%) | 0.39 | −1.89 | 2.00 |

TABLE 17
Wound Healing Clinical Outcomes

| | Placental | | |
|---|---|---|---|
| | Product | Control | |
| Wound Healing | (n = 50) | (n = 47) | p-value |
| Healed wounds, (N, %) | 31 (62%) | 10 (21%) | <0.001 |
| Median time to wound closure (days) | 42.0 | 69.5 | 0.019 |
| 50% wound area reduction at | 31 (62%) | 19 (40.4%) | 0.035 |
| Day 28 (N, %) | | | |
| Median study visits (Single Blind | 6 | 12 | <0.001 |
| Phase) | | | |

TABLE 18
Safety Outcomes

| | Placental | | | | |
|---|---|---|---|---|---|
| | Product | | Control | | |
| Adverse Events | (n = 50) | | (n = 47) | | p-value |
| Subjects experiencing at least one | 22 | (44%) | 31 | (66%) | 0.031 |
| adverse event^{#} (N, %) | | | | | |
| Subjects with an infection (N, %) | 13 | (26%) | 21 | (44.7%) | 0.055 |
| Subjects with a skin or | 7 | (14%) | 8 | (17%) | NS |
| subcutaneous tissue disorder | | | | | |
| (N, %) | | | | | |
| Subjects with injury, poisoning and | 5 | (10%) | 7 | (14.9%) | NS |
| procedural complications (N, %) | | | | | |
| Subjects with general disorders | 4 | (8%) | 3 | (6.4%) | NS |
| (N, %) | | | | | |
| Subjects with musculoskeletal and | 4 | (8%) | 2 | (4.3%) | NS |
| connective tissue disorders (N, %) | | | | | |
| Subjects with a wound related | 9 | (18%) | 17 | (36.2%) | 0.044 |
| infection (N, %) | | | | | |
| Subjects with a Serious Adverse | 4 | (8%) | 10 | (21.3%) | 0.084 |
| Event due to wound infection | | | | | |
| (N, %) | | | | | |
| Subjects having an amputation due | 0 | (0%) | 1 | (2.1%) | NS |
| to an adverse event (N, %) | | | | | |

^{#}Included overall number of subjects experiencing at least one adverse event and those with at least 5% adverse events

TABLE 19
Comparison of Standard of Care Among Multi-center, Controlled Wound
Care Trials

| | Grafix ® (amniotic | | |
|---|---|---|---|
| | placental product) | Dermagraft ® | Apligraf ® |
| Mean Wound Size | 3.7 cm² | 2.3 cm² | 3.0 cm² |
| Healed % Treatment vs. Control | 62% vs. 21%* | 30% vs. 18%* | 56% vs. 38%* |
| Time to closure | 42 vs. 70 days* | Not stated | 65 vs. 90 days* |
| All Adverse Events | 44% vs. 66%* | 67% vs. 73% | Not stated |
| Infection - wound related | 18% vs. 36.2%* | 19% vs. 32%* | 22% vs. 32% |
| Off-loading | Walking boot or | Therapeutic shoes | Custom sandal |
| | Post-op shoe | and custom insoles | |
| | | or healing sandals | |
| Debridement | every visit | ad lib | ad lib |

*p < 0.05

### Example 23

### Cryopreserved Membranes in Methods of Tendon Repair

Cryopreserved membrane is maintained at −80° C. (±5° C.) until it is ready for implantation. The frozen membrane is thawed and rinsed using a protocol provided the packaging insert. Additional information can be found in the Grafix Preparation Guide provided by Osiris Therapeutics. After thawing, a rinse step is performed and the graft may be held in the rinse basin for up to 1 hour prior to use. Once the membrane is thawed and rinsed, it is ready for implantation.

Depending on whether the cryopreserved membrane has a directionality (e.g., amniotic membrane) the packaging may indicate directionality of the membrane (e.g., which side is the epithelial or non-adherent side and/or which side is the stromal, or adherent side of the membrane). When using an amniotic membrane the stromal layer may be placed in contact with the repaired tissue surface.

The following technique provides an example that is expected to prove successful when using cryopreserved membranes disclosed herein for post tendon repair covering.

### Preparation of Membrane for Application

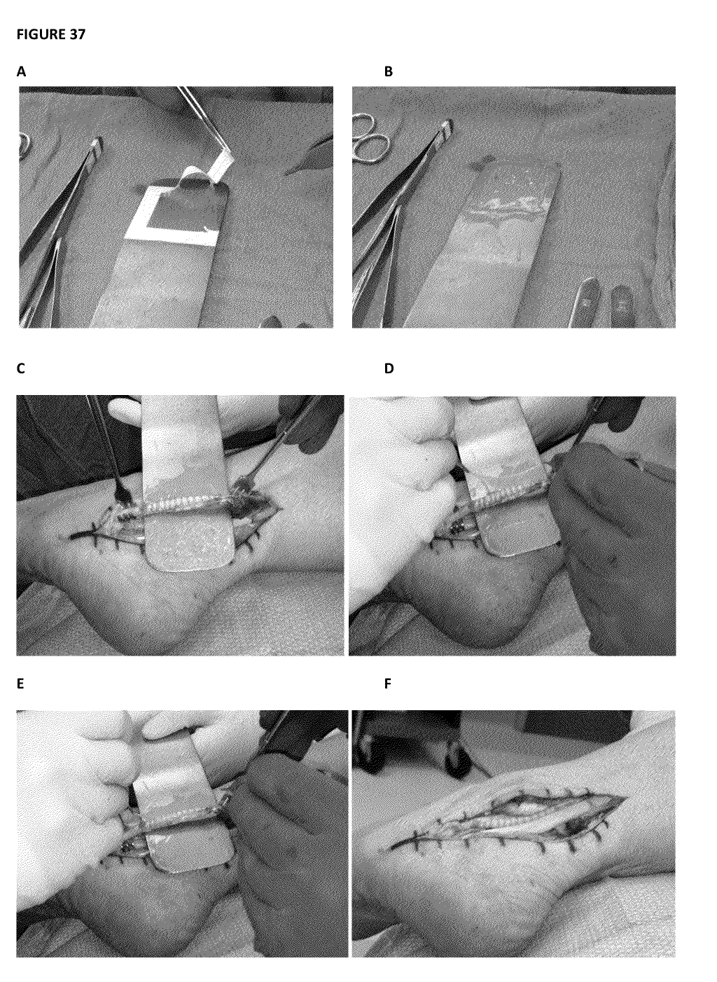

Prior to starting the application process, any irrigation of the wound and repair sites should be completed and any suction performed. This ensures the membrane will not be captured in the suction or disrupted after application. Any mounting support included with the cryopreserved membrane (e.g., nitrocellulose) is carefully removed after thawing is complete. When a membrane comprising a cryopreserved amniotic membrane on a nitrocellulose support is provided the preparative method may include:
1. Utilizing a 2-inch ribbon (malleable), place the epithelial side of the membrane in contact with the ribbon. In this position, the stromal side of the membrane is exposed with the nitrocellulose frame on top of the membrane.
2. Utilizing a single or two atraumatic forceps, and while stabilizing the membrane (e.g., with a finger), slowly begin pulling the nitrocellulose frame away from the membrane. It may be desirable to keep the membrane in contact with the ribbon at all times to prevent balling/contraction and to ensure appropriate identification of the stromal layer.
3. Once the backing is removed, carefully smooth out the membrane utilizing the back of the forceps, a finger or freer-elevator. The membrane is now stromal side up and ready to be transferred to the tendon. See, e.g., FIGS. 37 A-B

### Transferring Membrane to Tendon

After surgical repair of the tendon, the repaired tendon segment is carefully retracted to allow the ribbon to be passed under the repair site. The stromal side of the membrane will now be facing the repaired tendon segment. (FIG. 37 C). Utilizing atraumatic forceps, one entire side of the membrane will be carefully lifted to lay it over the tendon segment. The membrane (adherent side) will naturally adhere to the tendon but can be smoothed out and repositioned as needed. (FIG. 37 D). While stabilizing the applied membrane with either forceps or another instrument, the remaining portion of the membrane is wrapped in the opposite direction enveloping the remaining tendon segment. Any extra membrane may be allowed to overlap the previously applied section of membrane. (FIG. 37 E). This application will completely enclose the repaired tendon segment in a barrier sheath. Because of the stromal layer's adhering properties, and the potential inflammatory response from any resorbable suture, such sutures may not be recommended to keep the graft in place.

While tendon is still retracted, the ribbon is carefully removed and the tendon will be replaced back into the sheath. The tendon sheath is closed with careful attention not to inadvertently incorporate the membrane in the closure. (FIG. 37 F). If irrigation or suction proves necessary, acute observation of the membrane will be necessary to avoid membrane disruption or loss, or careful mopping with Ray-tec sponge may be recommended. Limiting the use of suction is expected to reduce the likelihood of disrupting the membrane.

### Discussion

Surgical reconstruction of tendon rupture is commonplace in most foot and ankle surgical practices. One of the most common postoperative complications is adhesion of the repaired tendon to the surrounding soft tissue structures. Cryopreserved membranes as disclosed herein, e.g., membranes comprising cryopreserved amniotic membrane, has been shown to have naturally anti-adhesive and anti-inflammatory properties. It is therefore ideally suited to employ in areas where inflammation and the resulting increase in circatrix formation would be detrimental. The membrane will not necessarily provide increased structural integrity, and as such, the isolated use of the membranes in the repair of tendon ruptures is appropriate when structural reinforcement is not required, or when using the membrane in combination with another product that provides structural reinforcement. A recent study by has shown that when the cryopreserved membrane was used along with an acellular dermal graft and PRP the rate of host incorporation of the dermal grafts was improved. The study suggests micro-anatomic remodeling of the graft into the physiologic identical tissue of the grafted site. Furthermore, an increased rate of incorporation, tissue differentiation and remodeling was observed. These features allow for the use of the cryopreserved membranes in applications relating to inhibition/prevention of tissue adhesion, inhibition/prevention of scarring during the postoperative period, and tendon repair.

### Example 23.1

### Cryopreserved Membranes in Methods of Tendon Repair

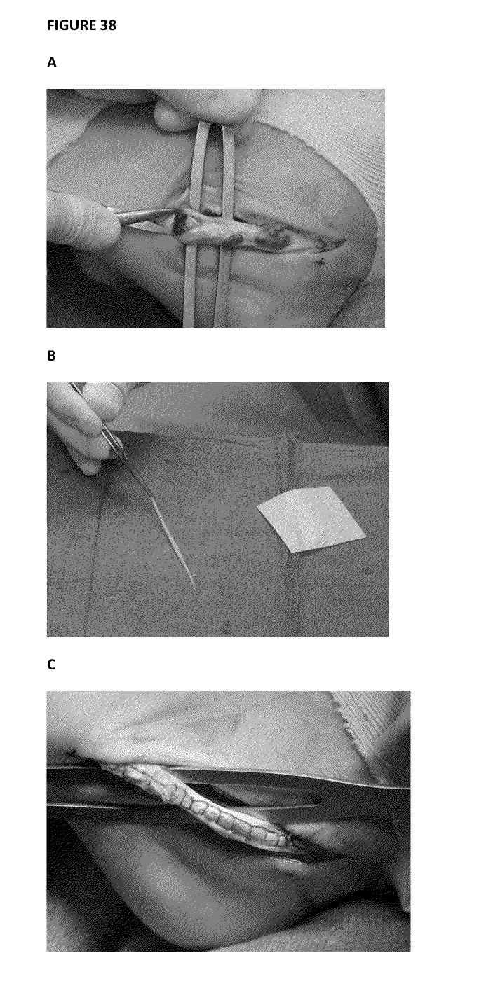

A 72 year old female patient presents with painful nodules and chronic tendinosis. The patient undergoes surgery to remove the nodules (FIG. 38 A). A cryopreserved membrane may be thawed as discussed above and rolled into a needle-like form (FIG. 38 B). The membrane is then inserted inside the tendon, and the tendon is closed with sutures. (FIG. 38 C). It is expected that the membrane will promote accelerated wound healing and tissue repair, as well as prevent adhesion to the repaired tendon.

### REFERENCES

Chang, Z., Y. Kishimoto, et al. (2014). “TGF-beta3 modulates the inflammatory environment and reduces scar formation following vocal fold mucosal injury in rats.” Dis Model Mech 7(1): 83-91.
Ferguson, M. W., J. Duncan, et al. (2009). “Prophylactic administration of avotermin for improvement of skin scarring: three double-blind, placebo-controlled, phase I/II studies.” Lancet 373(9671): 1264-1274.
Ishida, Y., T. Kondo, et al. (2004). “The essential involvement of cross-talk between IFN-gamma and TGF-beta in the skin wound-healing process.” J Immunol 172(3): 1848-1855.
Jiang, N., N. S. Tan, et al. (2007). “Respiratory protein-generated reactive oxygen species as an antimicrobial strategy.” Nat Immunol 8(10): 1114-1122.
Kim, W. S., B. S. Park, et al. (2008). “Evidence supporting antioxidant action of adipose-derived stem cells: protection of human dermal fibroblasts from oxidative stress.” J Dermatol Sci 49(2): 133-142.
Maxson, S., E. A. Lopez, et al. (2012). “Concise review: role of mesenchymal stem cells in wound repair.” Stem Cells Transl Med 1(2): 142-149.
Taylor, M. J., and Bank, H. L. (1998). “Function of Lymphocytes and Macrophages after Cryopreservation by Procedures for Pancreatic Islets: Potential for Reducing Tissue Immunogenicity” Cryobiology 25:1-17
Trengove, N. J., M. C. Stacey, et al. (1999). “Analysis of the acute and chronic wound environments: the role of proteases and their inhibitors.” Wound Repair Regen 7(6): 442-452.
Wang, X. Y., J. G. Crowston, et al. (2007). “Interferon-alpha and interferon-gamma sensitize human tenon fibroblasts to mitomycin-C.” Invest Ophthalmol V is Sci 48(8): 3655-3661.

1-79. (canceled)

80. A membrane comprising cryopreserved amniotic membrane having one or more tissue components, wherein after cryopreservation and subsequent thawing the amniotic membrane comprises:
A) tissue cells, wherein said tissue cells are native to the amniotic membrane and greater than 40% of said tissue cells are viable;
B) one or more therapeutic factors that are native to the amniotic membrane;
C) extracellular matrix that is native to the amniotic membrane; and
D) depleted amounts of one or more types of functional immunogenic cells.

81. The membrane of claim 80, wherein the one or more tissue components is present in an amount effective to:
(i) reduce the amount or activity of pro-inflammatory cytokines;
(ii) increase the amount or activity of anti-inflammatory cytokines;
(iii) reduce the amount or activity of reactive oxygen species;
(iv) increase the amount or activity of antioxidant agents;
(v) reduce the amount or activity of proteases;
(vi) increase cell proliferation;
(vii) increase angiogenesis; or
(viii) increase cell migration.

82. The membrane according to claim 80, wherein the amniotic membrane is fixed to a delivery substrate.

83. The membrane according to claim 35, wherein the delivery substrate comprises nitrocellulose.

84. The membrane according to claim 80, wherein the cryopreserved amniotic membrane is stored for an extended period of time prior to subsequent thawing.

85. The membrane according to claim 84, wherein the extended period of time is from about 6 to at least about 36 months.

86. The membrane according to claim 84, wherein the viability of the tissue cells is substantially maintained upon thawing.

87. The membrane of claim 80, wherein the viability of the tissue cells is substantially maintained for at least about 24 months when stored frozen.

88. The membrane according to claim 80, wherein the cryopreserved amniotic membrane can be thawed and ready for use within 30 minutes of the start of a thawing method.

89. A method of treating a wound on a subject comprising administering to the site of the wound the membrane of claim 80.

90. The method according to 89, wherein the wound is selected from the group consisting of selected from the group consisting of lacerations, scrapes, burns, incisions, punctures, wound caused by a projectile, an epidermal wound, skin wound, chronic wound, acute wound, external wound, internal wound, congenital wound, ulcer, pressure ulcer, diabetic ulcer, wound caused during or as an adjunct to a surgical procedure, venous skin ulcer, spinal injury, ocular wound, ocular injury, ear injury, otolaryngology wounds or injury, ocular injury, and avascular necrosis.

91. A method of promoting tissue repair and/or tissue regeneration in a subject comprising administering to the subject the membrane of claim 80, wherein the administration provides the viable therapeutic cells, extracellular matrix, and one or more therapeutic factors in an amount effective to promote tissue repair and/or tissue regeneration.

92. A method of treating or preventing tissue adhesion associated with a surgical procedure comprising administering the membrane of claim 80.

93. A method of accelerating wound healing in a subject having a wound in need of healing, the method comprising administering to the site of the wound a membrane according to claim 80;
wherein the administering is effective to promote wound closure by 12 weeks after an initial administering step.

94. A method of accelerating wound healing in a subject having a wound in need healing, the method comprising administering to the site of the wound a membrane according to claim 80;
wherein the administering is effective to promote wound closure by 5-6 weeks after an initial administering step.

95. A method of accelerating wound healing in a subject having a wound in need healing, the method comprising administering to the site of the wound a membrane according to claim 80;
wherein the administering is effective to promote reduction in wound size by 50% or more 28 days after an initial administering step.

96. A method of improving wound closure rate in a subject having a wound in need healing, the method comprising administering to the site of the wound a membrane according to claim 80;
wherein the administering is effective to improve wound closure rate by at least about 44% relative to standard wound treatment.

97. A method of treating a subject for a wound that is refractory to a prior wound healing treatment, the method comprising administering to the site of the wound a membrane according to claim 80;
wherein the administering is effective to promote wound closure by 12 weeks after an initial administering step.

98. A kit for treating a wound or a tissue defect comprising:
A) a membrane according to claim 80 in a pharmaceutically acceptable container; and
B) instructions for administering the membrane for treating the wound or the tissue defect.

99. A composition comprising the membrane of claim 31.
